SEQUENCE LISTING (326 sequences)

SEQ ID NO: 1 - protein, 417 aa, Saccharomyces cerevisiae
MTKTITVAHIQYDFKAVLEENDENDDEFYINVDKNLNEIKEHKIVVLGNSRGVDAGKGNTFEKVGSHLYKARLDGHDFLFNTIIRDGSKMLKRADYTAVDTAKLQMRRFILGTTEGDIKVLDSNFNLQREIDQAHVSEITKLKFFPSGEALISSSQDMQLKIWSVKDGSNPRTLIGHRATVTDIAIIDRGRNVLSASLDGTIRLWECGTGTTIHTFNRKENPHDGVNSIALFVGTDRQLHEISTSKKNNLEFGTYGKYVIAGHVSGVITVHNVFSKEQTIQLPSKFTCSCNSLTVDGNNANYIYAGYENGMLAQWDLRSPECPVGEFLINEGTPINNVYFAAGALFVSSGFDTSIKLDIISDPESERPAIEFETPTFLVSNDDEVSQFCYVSDDESNGEVLEVGKNNFCALYNLSNP

SEQ ID NO: 2 - protein, 267 aa, Saccharomyces cerevisiae
MSTSREDSVYLAKLAEQAERYEEMVENMKTVASSGQELSVEERNLLSVAYKNVIGARRASWRIVSSIEQKEESKEKSEHQVELICSYRSKIETELTKISDDILSVLDSHLIPSATTGESKVFYYKMKGDYHRYLAEFSSGDAREKATNASLEAYKTASEIATTELPPTHPIRLGLALNFSVFYYEIQNSPDKACHLAKQAFDDAIAELDTLSEESYKDSTLIMQLLRDNLTLWTSDMSESGQAEDQQQQQQHQQQQPPAAAEGEAPK

SEQ ID NO: 3 - protein, 744 aa, Saccharomyces cerevisiae
MTIDDSNRLLMDVDQFDFLDDGTAQLSNNKTDEEEQLYKRDPVSGAILVPMTVNDQPIEKSGDKMPLKFKLGPLSYQNMAFITAKDKYRLYPVRIPRLDTSKEFSAYVSGLFEIYRDLGDDRVFNVPTIGVVNSNFAKEHNATVNLAMEAILNELEVFIGRVKDQDGRVNRFYELEESLTVLNCLRTMYFILDGQDVEENRSEFIESLLNWINRSDGEPDEEYIEQVFSVKDSTAGKKVFETHYFWKLLNQLVLRGLLSQAIGCIERSDLLPYLSDTCAVSFDAVSDSIELLKQYPKDSSSTFREWKNLVLKLSQAFGSSATDISGELRDYIEDFLLVIGGNQRKILQYSRTWYESFCGFLLYYIPSLELSAEYLQMSLEANVVDITNDWEQPCVDIISGKIHSILPVMESLDSCTAAFTAMICEAKGLIENVFEGEKNSDDYSNEDNEMLEDLFSYRNGMASYMLNSFAFELCSLGDKELWPVAIGLIALSATGTRSAKKMVIAELLPHYPFVTNDDIEWMLSICVEWRLPEIAKEIYTTLGNQMLSAHNIIESIANFSRAGKYELVKSYSWLLFEASCMEGQKLDDPVLNAIVSKNSPAEDDVIIPQDILDCVVTNSMRQTLAPYAVLSQFYELRDREDWGQALRLLLLLIEFPYLPKHYLVLLVAKFLYPIFLLDDKKLMDEDSVATVIEVIETKWDDADEKSSNLYETIIEADKSLPSSMATLLKNLRKKLNFKLCQAFM

SEQ ID NO: 4 - protein, 444 aa, Saccharomyces cerevisiae
MNVVVCSGGTATNSLTPCFSNISILKGHELTYILPISDNGGSTSEILRIVGGPAIGDIRSRIVRLLQDEQLVELFGHRLPNDKLLAKKEWNEIVEGSHPIWKNISIEVKEMCRSFIIHMQAELLKKIKHSNPFQFESASIGNFFLTGARLFLGSLDASIELMMRIGRCSPLVHVIPCINTNHTHHISALLTNGEMITGQSQISHPSKSVPKDNSIAHSAKFIHLLGSYDDHLKILLDDEEEEAEEEYANPIYILPELKNSQLHFDKLDESQNLPAPVHRILYINPYGEEIKPMGNPRAISKVKKADMVVYSIGSLMTSLLPILILGNLAEVILESNNTKKVLLINNKYDREVFGLDGLHYVQMIIDSMSRAIAGYRQSKGVHSENDDFEWQDFITDIVYLKNGEIEIDETIFEKHSIRCHQIASSDKMESEELEKVLNQIGLKN

SEQ ID NO: 5 - protein, 362 aa, Saccharomyces cerevisiae
MLVPPANFGIAEEGIYRCSKVETLNLSFLETLNLKTAIFIGGQEPSKFFKDFFTRSSIKWIVLRMSDFSAAAVPVKSSSVSNANLYSNNNSTLSLQEEKKKSTANGSQNSTTGDPVIQEELAYHLTDNDDLMLIKSTCLKRTFKTLLNVDNYNVLLVDKTALVIGILRKIQKWNIASIINEYRLFSGKNRNYFAETFLEIINIEIEQEKDNKTIVDNKAKKLPLENNRTHSIEYKANSGKLIRVNEDDLCREPEVPQRLLTLINQIETKVKNNKVLQVSGVLGDDLKKTSSDLGIFGHRYRLAFNKKENGDYGYYKARGKDNVKIRIPCDSELPDWFKFQRDLWEKENVPEEHHFYREHIFT

SEQ ID NO: 6 - protein, 272 aa, Saccharomyces cerevisiae
MSQTREDSVYLAKLAEQAERYEEMVENMKAVASSGQELSVEERNLLSVAYKNVIGARRASWRIVSSIEQKEESKEKSEHQVELIRSYRSKIETELTKISDDILSVLDSHLIPSATTGESKVFYYKMKGDYHRYLAEFSSGDAREKATNSSLEAYKTASEIATTELPPTHPIRLGLALNFSVFYYEIQNSPDKACHLAKQAFDDAIAELDTLSEESYKDSTLIMQLLRDNLTLWTSDISESGQEDQQQQQQQQQQQQQQQQAPAEQTQGEPTK

SEQ ID NO: 7 - protein, 273 aa, Saccharomyces cerevisiae
MSNPFDLLGNDVEDADVVVLPPKEIVKSNTSSKKADVPPPSADPSKARKNRPRPSGNEGAIRDKTAGRRNNRSKDVTDSATTKKSNTRRATDRHSRTGKTDTKKKVNQGWGDDKKELSAEKEAQADAAAEIAEDAAEAEDAGKPKTAQLSLQDYLNQQANNQFNKVPEAKKVELDAERIETAEKEAYVPATKVKNVKSKQLKTKEYLEFDATFVESNTRKNFGDRNNNSRNNFNNRRGGRGARKGNNTANATNSANTVQKNRNIDVSNLPSLA

SEQ ID NO: 8 - protein, 162 aa, Saccharomyces cerevisiae
MVMLHSKNVKGFLENTLKPYDLHSVDFKTSSLQSSMIITATNGGILSYATSNNDVPKNSINEINSVNNLKMMSLLIKDKWSEDENDTEEQHSNSCYPVEIDSFKTKIYTYEMEDLHTCVAQIPNSDLLLLFIAEGSFPYGLLVIKIERAMRELTDLFGYKLG

SEQ ID NO: 9 - protein, 528 aa, Saccharomyces cerevisiae
MQQNTSLYDSLNVTAAASTSEIKKAYRNAALKYHPDKNNHTEESKRKFQEICQAYEILKDNRLRALYDQYGTTDEVLIQEQQAQAQRQQAGPFSSSSNFDTEAMSFPDLSPGDLFAQFFNSSATPSSNGSKSSFNFSFNNSSTPSFSFVNGSGVNNLYSSSAKYNSNDEDHHLDRGPDIKHNLKCTLKELYMGKTAKLGLNRTRICSVCDGHGGLKKCTCKTCKGQGIQTQTRRMGPLVQSWSQTCADCGGAGVFVKNKDICQQCQGLGFIKERKILQVTVQPGSCHNQLIVLTGEGDEVISTKGGGHEKVIPGDVVITILRLKDPNFQVINYSNLICKKCKIDFMTSLCGGVVYIEGHPSGKLIKLDIIPGEILKPGCFKTVEDMGMPKFINGVRSGFGHLYVKFDVTYPERLEPENAKKIQNILANDKYIKAERSTMETADSDCYCDLEKSYDSVEEHVLSSFEAPNLNNEVIEDDDLGDLINERDSRKRNNRRFDESNINNNNETKRNKYSSPVSGFYDHDINGY

SEQ ID NO: 10 - protein, 298 aa, Saccharomyces cerevisiae
MSTSVPVKKALSALLRDPGNSHCADCKAQLHPRWASWSLGVFICIKCAGIHRSLGTHISKVKSVDLDTWKEEHLVKLIQFKNNLRANSYYEATLADELKQRKITDTSSLQNFIKNKYEYKKWIGDLSSIEGLNDSTEPVLHKPSANHSLPASNARLDQSSNSLQKTQTQPPSHLLSTSRSNTSLLNLQVSSLSKTTSNTSVTSSATSIGAANTKTGNRVGEFGQRNDLKKSILSLYSKPSAQTQSQNSFFTSTTPQPCNTPSPFVNTGITATNNNSMNSNSSSNISLDDNELFKNVWS

SEQ ID NO: 11 - protein, 743 aa, Saccharomyces cerevisiae
MDGPNFAHQGGRSQRTTELYSCARCRKLKKKCGKQIPTCANCDKNGAHCSYPGRAPRRTKKELADAMLRGEYVPVKRNKKVGKSPLSTKSMPNSSSPLSANGAITPGFSPYENDDAHKMKQLKPSDPINLVMGASPNSSEGVSSLISVLTSLNDNSNPSSHLSSNENSMIPSRSLPASVQQSSTTSSFGGYNTPSPLISSHVPANAQAVPLQNNNRNTSNGDNGSNVNHDNNNGSTNTPQLSLTPYANNSAPNGKFDSVPVDASSIEFETMSCCFKGGRTTSWVREDGSFKSIDRSLLDRFIAAYFKHNHRLFPMIDKIAFLNDAATITDFERLYDNKNYPDSFVFKVYMIMAIGCTTLQRAGMVSQDEECLSEHLAFLAMKKFRSVIILQDIETVRCLLLLGIYSFFEPKGSSSWTISGIIMRLTIGLGLNRELTAKKLKSMSALEAEARYRVFWSAYCFERLVCTSLGRISGIDDEDITVPLPRALYVDERDDLEMTKLMISLRKMGGRIYKQVHSVSAGRQKLTIEQKQEIISGLRKELDEIYSRESERRKLKKSQMDQVERENNSTTNVISFHSSEIWLAMRYSQLQILLYRPSALMPKPPIDSLSTLGEFCLQAWKHTYTLYKKRLLPLNWITLFRTLTICNTILYCLCQWSIDLIESKIEIQQCVEILRHFGERWIFAMRCADVFQNISNTILDISLSHGKVPNMDQLTRELFGASDSYQDILDENNVDVSWVDKLV

SEQ ID NO: 12 - protein, 256 aa, Saccharomyces cerevisiae
MAPGKKVAPAPFGAKSTKSNKTRNPLTHSTPKNFGIGQAVQPKRNLSRYVKWPEYVRVQRQKKILSIRLKVPPTIAQFQYTLDRNTAAETFKLFNKYRPETAAEKKERLTKEAAAVAEGKSKQDASPKPYAVKYGLNHVVALIENKKAKLVLIANDVDPIELVVFLPALCKKMGVPYAIVKGKARLGTLVNQKTSAVAALTEVRAEDEAALAKLVSTIDANFADKYDEVKKHWGGGILGNKAQAKMDKRAKNSDSA

SEQ ID NO: 13 - protein, 423 aa, Saccharomyces cerevisiae
MTKAYTIVIKLGSSSLVDESTKEPKLSIMTLIVETVTNLKRMGHKVIIVSSGGIAVGLDALNIPHKPKQLSEVQAIAAVGQGRLIARWNMLFSQYGEQTAQILLTRNDILRWNQYNNARNTINELLAMGVIPIVNENDTLSISEIEFGDNDTLSAITAALVGADFLFLLTDVDCLYTDNPRTNPDARPIVLVPELSEGLPGVNTSSGSGSEVGTGGMRTKLIAADLASNAGIETIVMKSDRPEYVPKIVDYIQHHFRPPRHIGNGTQQQFLDLQDTELEQLRRYDVPMHTKFLANDNKHKLKNREFWILHGLITKGAIIIDENSYDKLLSKDMASLTPNAVIEVRDNFHELECVDLKIGQRLPNGELDISKPIQSVGCVRSNYTSLELAKIKGLPSEKIHDVLGYSVSEYVAHRENIAFPPQF

SEQ ID NO: 14 - protein, 246 aa, Saccharomyces cerevisiae
MNIQSNNSPNLSNNIVSKQVYYAHPPPTIDPNDPVQISFPTTEVVGHGSFGVVFATIIQETNEKVAIKKVLQDKRFKNRELEIMKMLSHINIIDLKYFFYERDSQDEIYLNLILEYMPQSLYQRLRHFVHQRTPMSRLEIKYYMFQLFKSLNYLHHFANVSRVFKKEDDQTVEFLADVLKYDPLERFNALQCLCSPYFDELKLDDGKINQITTDLKLLGFDENVELGHLSPDELSSVKKKLYPKSK

SEQ ID NO: 15 - protein, 928 aa, Saccharomyces cerevisiae
MFTSQRQLRQNGSPMSSSRSSQHSSGTASPISDSPASNRSYGRDLRGLMGIDIPANEPAFNRANSSDTIYFRPKKIYKMEHEHPSRSTLVQLQTRSQPDDVASSQVNPEGGTDDLELGDPCGNQSLYTIGAEYVPDLDFTKLVNEWQKSTEDLYEFRSSATPQVQIKDSGKGNYELWSSPDAILTQNKLRRDSFSQENSDSLSPEDSILSRNLHSKVKPIPLPRNSQQIFTPLSNLEAERRSSYTTSSNNNSITQNNKFSFAKLKYSLPTQSSAVPASFDSNASSLNFLPTTTLSTLSELQISPNDMMDLIQKLPRNFLNLPYTQRKKVIIEHAPSHDYKAMMSLVKKFMLTSSRSNFSLAGFANNASVSQATANDDNINSRNTPNNSNDTYVNTRPLQRSRHGSIASQFLSSFSPSMTSIAKMNSNPLSGSAGGSARPDDKGMEILGHRLGKIIGFGAWGIIRECFDIETGVGRVIKIVKFKGHQNIKKHVLREVAIWRTLKHNRILPLLDWKLDDNYAMYCLTERINDGTLYDLVISWDEFKRSKIPFAERCRLTIFLSLQLLSALKYMHSKTIVHGDIKLENCLLQKEGKKSDWKVFLCDFGMSCHFDEKHVYRNDTFDENLSSGNSHRKRKSIEQTNLIKYPTTNFLPDDRTNDFDASENLKYQFENRKHQPFTPKGMVSSSSHSLKHLNQPSSSSSSNLFHKPASQPQPQHRSPFHGRHKTTDFSNLEPEPSKYIGSLPYASPELLRYSDARRSKSVEMHIYDSPDSSQSEISAASSSSSNLSSLSSSTKASAVTNSGVTTSSPSGSSTDFPCIVSPLGPASDIWALGVMLYTMLVGKLPFNHEFEPRLRSLIKVGEFDRFSLAQVCKFDRKKNEGTIGQGLYDTVIGCLTIDLDKRWKLKRIEEVLQNEMNLSEAIHDNNGS

SEQ ID NO: 16 - protein, 1062 aa, Saccharomyces cerevisiae
MSENTTAPSDNITNEQRLPSGPKDDVDTLALTSAQNQANSLRKLDTDANAKALPSITDIPVSDDSDIKRQVGSGFGSNPLHIKDSEAFPHSSIEALKEGMDKVTKQCNDLKTALLSKDTSLTDSVQDLFNSLKVLSHNQSVLENKLDDVMKNQVNTDILVNNLNERLNKLSTMLQNTSKVNHSNLLIENSSNNTSSQHNTSSSRRGPGRPRKDASTSTMNKLVSNAASVNLKSASNQGAPFSPVNITLPTAVVQTSKSKRYFVEPSTKQESLLLSAPSSSRDDADMSLTSVPQRTNNENGKERPSTANSSSITPTPVTPNNLIQIKRKRGRPPKKRTVETMISNSTDTIDKSDASNRIKNEIPINSLLPSSKFHQIPSSPSNPVSQPAPVRTSRSATQEIDIKSLELASLISTNGDPNAEDSNTTDTVHNNVEGKVNVEENKTEKEKIITIKSSSENSGNNTTNNNNTDNVIKFSANSDINSDIRRLMVNDQFSLSYDASGNITVKLPPVSSPAAATAAAAAAVTSEMNRQQRELDKRRDSREKMLVNMKYNDRDKAKSFMESNKKLLKAMKEEERRKRMTSIIHDNHLNLNLNEISTRSKIKSAEKPTTKGSSMSPKPRSASISGISDHQQEGYQPLEQEKLVDIDNEGSNANSDSLKMGLTISAADTVHKVGIQSMLNSGEEAITKENAEYERKTPGDEETTTFVPLENSQPSDTIRKRTAGDDGALDQTENTSISPKKRRTEDHTKGEEDEGERGVGNSGTLATVENVSGDISADLSKGTSSIHNDTESANDSSNGNGNLGLGTESRNTLLTATPIELICREGFFYRRDIPDVPITTGAYLEFKFKAKEEELINSSINEEDYAAKSKHEKMNAHFFKPDIQEETELAFEILSKTTLTEKYVNSLEYFLMEFRWENKLVGLGLKLRESKRTWQRRKALFALFEFWRDQSRDKRRFHNYTILHAVKEMENYRIFINRSVSWFYNHITLLKMILYDLCDNVTTQWREWMFPHNETLPALGQDGINEDNLNETIDNMLIFDFLDDGSENNQVKYSRIIPPDIR

SEQ ID NO: 17 - protein, 256 aa, Saccharomyces cerevisiae
MNTSSRITYFIIGGSRGIGFNLVKILSASTGNTVITSIRGSPSLPKNKQVEDLAKIRKNIHIVQLDLTKDESIGNIADEIKKTPFFLGIDIFIACSAVSDSYYKVLETPKSVWLNHYSTNALGPILALQKVYPLLLLKKTRKIFFISSVAGSINAFVPLSVSAYGQSKAALNYAVKTLSFELKPEGFTVVAFHPGMVSTDMGQYGLDHFKEKNIDISGVNIITPEESASALIDVFRKILPEDNGKFFNYDGSEGVF

SEQ ID NO: 18 - protein, 48 aa, Saccharomyces cerevisiae
MSYTRVDHPTGKMACHLRQILASPLFFANYVLHAAIHYPSSDIRGDIL

SEQ ID NO: 19 - protein, 148 aa, Saccharomyces cerevisiae
MLVKTISRTIESESGFLQPTLDVIATLPADDRSKKIPISLVVGFKQEASLNSSSSLSCYYYAIPLMRDRHINLKSGGSNVVGIPLLDTKDDRIRDMARHMATIISERFNRPCYVTWSSLPSEDPSMLVANHLYILKKCLDLLKTELGE

SEQ ID NO: 20 - protein, 493 aa, Saccharomyces cerevisiae
MSNDEGETFATEQTTQQVFQKLGSNMENRVCFDCGNKNPTWTSVPFGVMLCIQCSAVHRNMGVHITFVKSSTLDKWTINNLRRFKLGGNHKARDFFLKNNGKQLLNTANVDAKTKYTSPVAKKYKIHLDKKVQKDMELYPSELVLNGQDSSDSPLDTDSDASRSTSKENSVDDFFSNWQKPSSNSSSKLNVSTGSLAPKNNTTGSTPKTTVTKTRSSILTASRKKPVLNSQDKKKHSILSSSRKPTRLTAKKVDKSQAEDLFDQFEKEAQQEKEDEFTNSSSSTKIRQNDYDSQFMNNSKGNNNNSIDDINTQPDEFNDFLNDTSNSFDTTRKEQQDTLTPKFAKLGFGMTMNDANDLAKQQKESQKIAQGPRYTGRIAERYGTQKAISSDQLFGRGSFDEAANREAHDKLKTFDNATSISSSSYFGEDKEVDEFGNPINSSGSGAGNFDGRNSNNGFIDFNASADDELQMLRDVVEQGAEKLGSYLRDYLRK

SEQ ID NO: 21 - protein, 315 aa, Saccharomyces cerevisiae
MGRTFIHASKIKHAARKRKHHSNFRTLIKLLNNDAYKIESSKPLKNGKLFKYWKNRRRLFSKIDSASIYMTDELWFSVTPERIACFLANFVKACMPNAERILDVFCGGGGNTIQFAMQFPYVYGVDYSIEHIYCTAKNAQSYGVDDRIWLKRGSWKKLVSKQKLSKIKYDCVFGSPPWGGPEYLRNDVYDLEQHLKPMGITKMLKSFLKLSPNVIMFLPRNSDLNQLSRATRKVLGPFAKCKVLYVKENGYMKGIFCMWGECFFNYEPASTENSRRESSEKEELSSENEELSKRKKHESTTTTKDNTVDIYDVNG

SEQ ID NO: 22 - protein, 239 aa, Saccharomyces cerevisiae
MVSMIHIVVFLLAITTMFEILPLITVPVTKYLSLSSFRNHYYGLFGWCVRGQNQELMCTKMKIGYDSTDVDSSGHVLTLPSNSKVVVSNLLVVHPISLAFTGTLLILAVIIMVTPLGDSPEMLLFTALFSLPTFMLCLLCFLVDILLFISKLDWPGWLMLAATISVALCCSMLWVMRRVVSVKKYESQQSIAHACSMEQYSISDIYQSKQNGNSSEYEVAPTHTDSLIAPEVTYRGFIE

SEQ ID NO: 23 - protein, 396 aa, Saccharomyces cerevisiae
MYEMSGIDSLFHDRVVHDYSHTSEQVIVVYISSAAGDNSWLHQWFKPGNLSDEERENILWVRLVNGTKECLLFKSIFPSSSAPSINILQNGLLECSIQGNSLSREQDPWETFINGLQSVFKGQVTKRKLFSKSNEEYQRVKRMIQNDKLERKYVFQNTNDPQRKPQKWKQLTVTDNVSYKSQKGFLAQNYCTLQLKLPNGYTISNTFPPQTKLHKVRMWLDYNCYDDGTPYLFHRNIPRVTLTRNDELKSLQELDLLPRSTLILEPLEANNKTFDYMEQSSLLHKVYSGLTSFWAKEPEVDASSSRLGYQRLGTNVSNSANYSLQKLSSLDMVSDGGGGGGGDSMTPSAYTTPRMYPSNGTSQLRQNVSELNLSSNNSASNTKVRTLGYSNNNGNN

SEQ ID NO: 24 - protein, 450 aa, Saccharomyces cerevisiae
MAKDLLPKQAANEPSLKDCTCKRCLKLGASKEKKIRRKKKGEEKRERHYGNRRKLTFNFLKHTNMENTNYDVITSVGYLNEKYGLKKSHYIEKFIKCIHRKINIDVSKITDAYVNSLNPWVKVKLFLLLVTLSEKGGPEYWLDKTDGEKNSEASSTDNSLENSTKGADSAGSTALRDEMVKSHKNLFPTLTEQIIQHNINQDFTESTYDEDYVFSSIWANFMEGLINHYLEKVIVPYSEMKVCQQLYKPMMKIISLYNEYNELMVKSEKNGFLPSLQDSENVQGDKGEKESKDDAVSQERLERAQKLLWQAREDIPKTISKELTLLSEMYSTLSADEQDYELDEFVCCAEEYIELEYLPALVDVLFANCGTNNFWKIMLVLEPFFYYIEDVGGDDDEDEDNVDNSEGDEESLLSRNVEGDDNVVERHFKPDPRVITLEKICEVAARQKWI

SEQ ID NO: 25 - protein, 224 aa, Saccharomyces cerevisiae
MTLVTPLQFSTVQPNLYRGSYPREINLPFLRTLRLKYILSLTPEPLSTDPLMVKFCEENNIKTIHIKCQSERKADKTKPKIKRKKKTVPIEYDVVVRCVKFLIDKGHYPCYMHCTNGELIISLVVACMRKFSYWSTVSILNEFLVYNSSINIHERNFIENFNSEIEVDDLDIKDKVPWITVRYIARTATESKDELRVDDANASEKVARVSSVSNSLPKLKFHSM

SEQ ID NO: 26 - protein, 830 aa, Saccharomyces cerevisiae
MAIKSLESFLFERGLVGSYAIEALNNCTLGIDVNHYVSRLLTNKREQYLDAIGGFPTSLKMYLESDLKIFKDFNITPIFVFNGGLTYNQLEASGHFTAASASASISSTTTSSSGTNATTRSNTESVLLQRSRGWTQWNNLISSNQNSYIDQPIQPQEPFRHNTTIDSKAYQNDLIAYFIEHGYMYQVAPYSSWFQLAYLLNSAYIDAIYGPTDCLMLDCVDRFILGMEFPNKEFRFIDRSRVMKDLGCTHEEFIDIAMAVGNDLQPTTLPPLQIYPVPQLFDIALEMVLNTGTNFYAYQLSTTLQNDSKENIQNYQRGISALRYMPVLKDTGKVELFVQEIVVSEEDSEKNNKDGKKSNLSSPSSASSSASPATTVTKNASEKLTYEKSSTKEVRKPRDIPNDVHDFIGQMLPHEYYFYRSIGLVTGKLFDAIVTGVYPEEPPLGGGSSTSYRKLVSKSVEIFKNKEINLLTQPINRYYQIKQIKQVKWYAANEPTTLTNRMSPSMFETINHLIVKTETSDEKEFSISEFITTINGSSNMAKDFISEKVIFPNSVPIESKLNSPFNLLSTNFLRLLVLLEFFTFDFKEKLLEPTRWGEVFLKLNELNIDSKYHESVIIFLVFLKCDVLKLDEEVQPPAPSALSQATLRSYPEESLYVLLITRVLTLFQVDQKPSNYHGPIDKKTLIFRDHLSFIKENLNELFEAVLISSLTSGEFNRLSLDNFGWARKIVRYLPFKLDSPNTIMAMMWEFFLQKYLHNGNAKNDALSLVATEFNTYKSTPNLDEQFVESHRFLLEISKVMQELNAAKLIDENVFKLFTKAVEFTTTALSS

SEQ ID NO: 27 - protein, 455 aa, unidentified
MLRRALLLSSSAILAVKAQQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTAS

SEQ ID NO: 28 - protein, 754 aa, Saccharomyces cerevisiae
MAHEKFSIPENFTLAQSLQLLYSVVKNQYKNLADLIINSKGNKDTVTYGKIHKNLDTLLVYVNEGLRKIEKTYTLKKGLGNLVVDHPELRSIIEDFQILGQDIRIARRKAETLMAEGNGSPSLSSSSSVLGLGTGNGLRFPKLWRMGSKRDKLKEADEKEAKINKQADNIRRARKLEEEKKLGAKLQYERDLELQREKLIELKVKEKVEFEVAQKLEEERVKREEEERKHREQAEKKRISTLKHERKTNYKSRASLDNFSSSSKSSGKIDNSLIKRRSLDVVRTSGERVRTPVRKSMEAAEIGMAAQLAWSQYQNGANHSKVSNNGSHSNELQIRYKPTPPLKKRYDYKKPTVNRPIIKSPTLNRQNSKSSRNIPTNSKLKASKSNTNKVSRRNEQNLEPSSPVLVSATAVPAESKPMRSKSGTPDKESSASSSLDSRKEDILKSVQGVDRNACEQILNEILVTDEKVYWEDIAGLRNAKNSLKEAVVYPFLRPDLFKGLREPVRGMLLFGPPGTGKTMIAKAVATESNSTFFSVSASSLLSKYLGESEKLVRALFYMAKKLSPSIIFIDEIDSMLTARSDNENESSRRIKTELLIQWSSLSSATAQSEDRNNTLDSRVLVLGATNLPWAIDDAARRRFSRKLYIPLPDYETRLYHLKRLMAKQKNSLQDLDYELITEMTEGFSGSDLTSLAKEAAMEPIRDLGDKLMFADFDKIRGIEIKDFQNALLTIKKSVSSESLQKYEEWSSKFGSNGS

SEQ ID NO: 29 - protein, 312 aa, Saccharomyces cerevisiae
MSFHQQFFTLNNGNKIPAIAIIGTGTRWYKNEETDATFSNSLVEQIVYALKLPGIIHIDAAEIYRTYPEVGKALSLTEKPRNAIFLTDKYSPQIKMSDSPADGLDLALKKMGTDYVDLYLLHSPFVSKEVNGLSLEEAWKDMEQLYKSGKAKNIGVSNFAVEDLQRILKVAEVKPQVNQIEFSPFLQNQTPGIYKFCQEHDILVEAYSPLGPLQKKTAQDDSQPFFEYVKELSEKYIKSEAQIILRWVTKRGVLPVTTSSKPQRISDAQNLFSFDLTAEEVDKITELGLEHEPLRLYWNKLYGKYNYAAQKV

SEQ ID NO: 30 - protein, 471 aa, Saccharomyces cerevisiae
MSICPHIQQVFQNEKSKDGVLKTCNAARYILNHSVPKEKFLNTMKCGTCHEINSGATFMCLQCGFCGCWNHSHFLSHSKQIGHIFGINSNNGLLFCFKCEDYIGNIDLINDAILAKYWDDVCTKTMVPSMERRDGLSGLINMGSTCFMSSILQCLIHNPYFIRHSMSQIHSNNCKVRSPDKCFSCALDKIVHELYGALNTKQASSSSTSTNRQTGFIYLLTCAWKINQNLAGYSQQDAHEFWQFIINQIHQSYVLDLPNAKEVSRANNKQCECIVHTVFEGSLESSIVCPGCQNNSKTTIDPFLDLSLDIKDKKKLYECLDSFHKKEQLKDFNYHCGECNSTQDAIKQLGIHKLPSVLVLQLKRFEHLLNGSNRKLDDFIEFPTYLNMKNYCSTKEKDKHSENGKVPDIIYELIGIVSHKGTVNEGHYIAFCKISGGQWFKFNDSMVSSISQEEVLKEQAYLLFYTIRQVN

SEQ ID NO: 31 - protein, 686 aa, Saccharomyces cerevisiae
MTDITPVQNDVDVNGNNVNDDVSSNLKRPIDQGDPSNGLAEEENPANNQLHLKKARLDGDALTSSPAGLAENGIEGATLAANGENGYNATGSGAEDEQQGLKKEEGGQGTKQEDLDENSKQELPMEVPKEPAPAPPPEPDMNNLPQNPIPKHQQKHALLAIKAVKRLKDARPFLQPVDPVKLDIPFYFNYIKRPMDLSTIERKLNVGAYEVPEQITEDFNLMVNNSIKFNGPNAGISQMARNIQASFEKHMLNMPAKDAPPVIAKGRRSSAQEDAPIVIRRAQTHNGRPKRTIHPPKSKDIYPYESKKPKSKRLQQAMKFCQSVLKELMAKKHASYNYPFLEPVDPVSMNLPTYFDYVKEPMDLGTIAKKLNDWQYQTMEDFERDVRLVFKNCYTFNPDGTIVNMMGHRLEEVFNSKWADRPNLDDYDSDEDSRTQGDYDDYESEYSESDIDETIITNPAIQYLEEQLARMKVELQQLKKQELEKIRKERRLARGSKKRGKRSKGRSGSKNASSKGRRDKKNKLKTVVTYDMKRIITERINDLPTSKLERAIDIIKKSMPNISEDDEVELDLDTLDNHTILTLYNTFFRQYESSSGASNGLDGTSGVTRDASSLSPTSAGSRKRRSKALSQEEQSRQIEKIKNKLAILDSASPLSQNGSPGQIQSAAHNGFSSSSDDDVSSESEEE

SEQ ID NO: 32 - protein, 959 aa, Saccharomyces cerevisiae
MFGNNRPMFGGSNLSFGSNTSSFGGQQSQQPNSLFGNSNNNNNSTSNNAQSGFGGFTSAAGSNSNSLFGNNNTQNNGAFGQSMGATQNSPFGSLNSSNASNGNTFGGSSSMGSFGGNTNNAFNNNSNSTNSPFGFNKPNTGGTLFGSQNNNSAGTSSLFGGQSTSTTGTFGNTGSSFGTGLNGNGSNIFGAGNNSQSNTTGSLFGNQQSSAFGTNNQQGSLFGQQSQNTNNAFGNQNQLGGSSFGSKPVGSGSLFGQSNNTLGNTTNNRNGLFGQMNSSNQGSSNSGLFGQNSMNSSTQGVFGQNNNQMQINGNNNNSLFGKANTFSNSASGGLFGQNNQQQGSGLFGQNSQTSGSSGLFGQNNQKQPNTFTQSNTGIGLFGQNNNQQQQSTGLFGAKPAGTTGSLFGGNSSTQPNSLFGTTNVPTSNTQSQQGNSLFGATKLTNMPFGGNPTANQSGSGNSLFGTKPASTTGSLFGNNTASTTVPSTNGLFGNNANNSTSTTNTGLFGAKPDSQSKPALGGGLFGNSNSNSSTIGQNKPVFGGTTQNTGLFGATGTNSSAVGSTGKLFGQNNNTLNVGTQNVPPVNNTTQNALLGTTAVPSLQQAPVTNEQLFSKISIPNSITNPVKATTSKVNADMKRNSSLTSAYRLAPKPLFAPSSNGDAKFQKWGKTLERSDRGSSTSNSITDPESSYLNSNDLLFDPDRRYLKHLVIKNNKNLNVINHNDDEASKVKLVTFTTESASKDDQASSSIAASKLTEKAHSPQTDLKDDHDESTPDPQSKSPNGSTSIPMIENEKISSKVPGLLSNDVTFFKNNYYISPSIETLGNKSLIELRKINNLVIGHRNYGKVEFLEPVDLLNTPLDTLCGDLVTFGPKSCSIYENCSIKPEKGEGINVRCRVTLYSCFPIDKETRKPIKNITHPLLKRSIAKLKENPVYKFESYDPVTGTYSYTIDHPVLT

SEQ ID NO: 33 - protein, 258 aa, Saccharomyces cerevisiae
MGSRRYDSRTTIFSPEGRLYQVEYALESISHAGTAIGIMASDGIVLAAERKVTSTLLEQDTSTEKLYKLNDKIAVAVAGLTADAEILINTARIHAQNYLKTYNEDIPVEILVRRLSDIKQGYTQHGGLRPFGVSFIYAGYDDRYGYQLYTSNPSGNYTGWKAISVGANTSAAQTLLQMDYKDDMKVDDAIELALKTLSKTTDSSALTYDRLEFATIRKGANDGEVYQKIFKPQEIKDILVKTGITKKDEDEEADEDMK

SEQ ID NO: 34 - protein, 439 aa, Saccharomyces cerevisiae
MVTKHQIEEDHLDGATTDPEVKRVKLENNVEEIQPEQAETNKQEGTDKENKGKFEKETERIGGSEVVTDVEKGIVKFEFDGVEYTFKERPSVVEENEGKIEFRVVNNDNTKENMMVLTGLKNIFQKQLPKMPKEYIARLVYDRSHLSMAVIRKPLTVVGGITYRPFDKREFAEIVFCAISSTEQVRGYGAHLMNHLKDYVRNTSNIKYFLTYADNYAIGYFKKQGFTKEITLDKSIWMGYIKDYEGGTLMQCSMLPRIRYLDAGKILLLQEAALRRKIRTISKSHIVRPGLEQFKDLNNIKPIDPMTIPGLKEAGWTPEMDALAQRPKRGPHDAAIQNILTELQNHAAAWPFLQPVNKEEVPDYYDFIKEPMDLSTMEIKLESNKYQKMEDFIYDARLVFNNCRMYNGENTSYYKYANRLEKFFNNKVKEIPEYSHLID

SEQ ID NO: 35 - protein, 270 aa, Saccharomyces cerevisiae
MTMRTNKKNKSSNIKMIRRVRHNLYNQNQGKLIVPNFFFFWHDMNSSGDNAGFEWWRRTMQYKTGIGLTPEEKTRYEDDSKARELKKECLKCYEYRDWMLKYSPTVRFMVQAITKLNKGSDSKFDDSKIICDYCPDWKGGGFHPELGILLCQNRLRDKWHLEDTLSHELIHYFDDLKWQIDWLNLKHHACSEIRASSLSGECRFWEEFKRRGFRTGFHVARGHQDCVRRRAIISVSGNPNCQSKEHAAKIVDEVWDSCFADTRPFDEIYR

SEQ ID NO: 36 - protein, 393 aa, Saccharomyces cerevisiae
MAKIMIPASKQPVYKKLGLLLVAVFTVYVFFHGAQYARGSAPSPKYSTVLSSGSGYKYSKVELPKYTGPREKATFVTLVRNRDLYSLAESIKSVEDRFNSKFNYDWVFLNDEEFTDEFKNVTSALVSGTTKYGVIPKEHWSFPEWIDEEKAAQVRKEMGEKRIIYGDSISYRHMCRFESGFFYRHPLMDDYDWYWRVEPDIKLHCDIDYDVFKFMKDNKKKYAFAISIKEYEATIPTLWETTRKFMEAHPELIHENNMLDFVSDDQGLSYNLCHFWSNFEIAALDLWRSPAYSAYFDYLDREGGFFYERWGDAPVHSIGAALFLDRSEIHHFGDIGYYHVPFHSCPIDTSIRLANKCDCDPSKDFTWHSYSCTTKFYNINKLPKPAGWQNHIG

SEQ ID NO: 37 - protein, 759 aa, Saccharomyces cerevisiae
MSSSSSTGYSKNNAAHIKQENTLRQRESSSISVSEELSSADERDAEDFSKEKPAAQSSLLRLESVVMPVIFTALALFTRMYKIGINNHVVWDEAHFGKFGSYYLRHEFYHDVHPPLGKMLVGLSGYLAGYNGSWDFPSGEIYPDYLDYVKMRLFNASFSALCVPLAYFTAKAIGFSLPTVWLMTVLVLFENSYSTLGRFILLDSMLLFFTVASFFSFVMFHNQRSKPFSRKWWKWLLITGISLGCTISVKMVGLFIITMVGIYTVIDLWTFLADKSMSWKTYINHWLARIFGLIIVPFCIFLLCFKIHFDLLSHSGTGDANMPSLFQARLVGSDVGQGPRDIALGSSVVSIKNQALGGSLLHSHIQTYPDGSNQQQVTCYGYKDANNEWFFNRERGLPSWSENETDIEYLKPGTSYRLVHKSTGRNLHTHPVAAPVSKTQWEVSGYGDNVVGDNKDNWVIEIMDQRGDEDPEKLHTLTTSFRIKNLEMGCYLAQTGNSLPEWGFRQQEVVCMKNPFKRDKRTWWNIETHENERLPPRPEDFQYPKTNFLKDFIHLNLAMMATNNALVPDPDKFDYLASSAWQWPTLNVGLRLCGWGDDNPKYFLLGTPASTWASSVAVLAFMATVVILLIRWQRQYVDLRNPSNWNVFLMGGFYPLLAWGLHYMPFVIMSRVTYVHHYLPALYFALIILAYCFDAGLQKWSRSKCGRIMRFVLYAGFMALVIGCFWYFSPISFGMEGPSSNFRYLNWFSTWDIADKQEA

SEQ ID NO: 38 - protein, 579 aa, Saccharomyces cerevisiae
MKYVVVSGGVISGIGKGVLASSTGMLMKTLGLKVTSIKIDPYMNIDAGTMSPLEHGECFVLDDGGETDLDLGNYERYLGVTLTKDHNITTGKIYSHVIAKERKGDYLGKTVQIVPHLTNAIQDWIERVAKIPVDDTGMEPDVCIIELGGTVGDIESAPFVEALRQFQFKVGKENFALIHVSLVPVIHGEQKTKPTQAAIKGLRSLGLVPDMIACRCSETLDKPTIDKIAMFCHVGPEQVVNVHDVNSTYHVPLLLLEQKMIDYLHARLKLDEISLTEEEKQRGLELLSKWKATTGNFDESMETVKIALVGKYTNLKDSYLSVIKALEHSSMKCRRKLDIKWVEATDLEPEAQESNKTKFHEAWNMVSTADGILIPGGFGVRGTEGMVLAARWARENHIPFLGVCLGLQIATIEFTRSVLGRKDSHSAEFYPDIDEKNHVVVFMPEIDKETMGGSMRLGLRPTFFQNETEWSQIKKLYGDVSEVHERHRHRYEINPKMVDELENNGLIFVGKDDTGKRCEILELKNHPYYIATQYHPEYTSKVLDPSKPFLGLVAASAGILQDVIEGKYDLEAGENKFNF

SEQ ID NO: 39 - protein, 310 aa, Saccharomyces cerevisiae
MSTNTEIIKNSDLQSLINDKRRLINEIKDFNKSIKPLEFESYQDYFLIKTFKKGISASGHVDIDSLRNKEYGIYYKKIKRNSTQEVGEPIPRNTSSSSSSTRSNSSADISDTEYSGENTPTTTGAASRRRRTRSRAIQRENSLPASLPSISEANANNDDVTISEINGSELPFPIPISEVENIDIASDITERDGIRRRSSRISERDKRRSQSRLGSEEDEEGDGHDGDEGETKIQDLYESLVPKILESRRRSDWILPPKARYTPEKQMRTKPSFKSIKINELVGNKRIRSILSRFEGGVAGIRKRDWDSTQ

SEQ ID NO: 40 - protein, 785 aa, Saccharomyces cerevisiae
MSVKEHNEEDIIGDELQNSRQLSIDCDSVKISLRNTYWTKDYTTGIKLFIKHMKRENDLLIKDIKFYNDFVNKFWKPTLNNLQKMEATNSMNSRLLEVMSKQFNIISTEQVERDCKIPLQELRDLNESFLREAENDLSSRYSAYIKDLVAAKEALIGCEKRVQSIYKLKKANTPVENSSSVFDNGKDSAPLTRLNFVCEFPYTLDERLKFEDCDQFMSFLQTLKGKVILEKSVFSVPGLSNQSFQGRSLIKELKKLEPRLNLSLFNIDRIGNEFIQLGIIQEYSLSFYSSKVSQFDQEKYYYWNSEVLATQESNGNAGNRKKKSYGELTHSDNEHEEKSNVSSIKTSISDWIRKVSQHDNDDCDAAGSTDMNKNEWKSLKQQLESSQDIFFSKCCQLEYSKVQLEKTIYDYCKNYSKMEDGIKRALESSNMMFQQKCEKFTDSPVCSLQEAQLPQETANADVRGFFLRDNGIPFRRWNILEASDPVDACKEISIKSEKFFCGSEINNELAALDTLGAIKIILRQIEKEPNANKVIQSWHRDIDFVRVSNLKRDLLGEFKGSKTTENTNSIITAHFFENSHSYVTNDLVGLIKLWLLELPDSLIPSNHYDDLIKAEKSLTSLCEQFPTSSLRFLQELANHFQLINSKYSLPPQTIQDLFRDNSDIDIPLAHHFVRRTGLQNPIDIKILSPTLSTFFINERTVETLQTLIANRITTATTATLTEPPTIIIKDTTAPIHSTPKPPPNDKDGHFIPRPFKTSSTPTTPERPKRKSGLFLPINVNDVPST

SEQ ID NO: 41 - protein, 182 aa, Saccharomyces cerevisiae
MGAVLSCCRNHSGEENEALLREQQAGYGSQGNANDEYDAEQMRLKEHEQKLLAREQELRDIVANTNDKLIDISMINNSGIVIQGTDLQEALDKRQQEEGGDSREDERSAGDDNLSGHSVPSSGSAQATTHQTAPRTNTFTLLTSPDSAKISKEQLKKLHSNILNEIFSQSQVNKPGPLTVPF

SEQ ID NO: 42 - protein, 238 aa, Saccharomyces cerevisiae
MTATITNKKSCSGSVEAGKTRLTTEWKPESQVPQYVKNELSKPHPNYILAFLNVVQQLKIQRRTGYLDLGIKECESISDHMYRLSIITMLIKDSRVNRDKCVRIALVHDIAESLVGDITPVDPIGKEEKHRREWETIKYLCNALIKPYNEIAAKEIMDDWLAYENVTSLEARYVKDIDKYEMLVQCFEYEREYKGTKNFDDFFGAVASIKTDEVKGWTSDLVVQRQKYFADLTQSITK

SEQ ID NO: 43 - protein, 818 aa, Saccharomyces cerevisiae
MAGNGKDKEVHESPSVSTLKLLGKRLFNSSSHTDNSSLLLSAEQLGNGRSLRKRPTSPSISGSGSGGNSPSSSAGARQRSASLHRRKNNASVGFSNGPVSSHKSSAALQDLIKHNNNPYLNSPSDILGTGTGIASTRDRDRAVLDREKEKERARNKERNTHHAGLPQRSNSMASHHFPNENIVYNPYGISPNHARPDTAFADTLNTNKENDLSFYMHDGNSKIRMLPLPIANPNDFLPEDMKQYSVHLTDNFVFDTDNKPIGSGGSSEVRKVKSSYRQKDVYALKKLNMIYHESPEKFYKRCSKEFIIAKHLSHNVHITKTFYLLKVPTTTYTTRGWGFIMELGVKDLFQLMERTGWKNVPFNEKYCLFKQVAQGIKFCHDNGIAHRDLKPENVLISKEGICKLTDFGISDWYHVIPHDYTSPVKTCQGMIGSPPYTPPEVMYFDAKKHYPEKFQKPYNPLAMDSYALGIMLITMINNIIPFIDSCNTDARFREFEVSYDNFINHQNPHFRDKGCHKPGPGSEYSLARNFKNTDATRIAWRLADPNPATRYTMDDLFNDPFFQQIETCVEPNDDDLVRVPELRKSTSTNDFSENSLDAPHDQEVIHTSNPFLKKETLTSKPRSMLEIAESPSLKQKSKVKDSAKTKTHDVGDEGGNESTKPKQQDKKENLKKDEVKNGDKDKVIEEATTTNVDSILEKPTPTSTKVEDNLSEDDSTMKELKSMLNSTPTTPTHNGPTPLPAKAGTQLDKRMSDLSLKSETPASTKNFSAPNVSSSSNSLRSLGSPSVSSSKKKKVIHHHLDITNSVTNMSSVSAFISR

SEQ ID NO: 44 - protein, 49 aa, Saccharomyces cerevisiae
MIQKPILLSSFLFLYIRALLHSIHPYIRTSVHLYTKKITSYNFLGVPFK

SEQ ID NO: 45 - protein, 678 aa, Saccharomyces cerevisiae
MGLLALGTPLQWFESRTYNEHIRDEGIEQLLYIFQAAGKRDNDPLFWGDELEYMVVDFDDKERNSMLDVCHDKILTELNMEDSSLCEANDVSFHPEYGRYMLEATPASPYLNYVGSYVEVNMQKRRAIAEYKLSEYARQDSKNNLHVGSRSVPLTLTVFPRMGCPDFINIKDPWNHKNAASRSLFLPDEVINRHVRFPNLTASIRTRRGEKVCMNVPMYKDIATPETDDSIYDRDWFLPEDKEAKLASKPGFIYMDSMGFGMGCSCLQVTFQAPNINKARYLYDALVNFAPIMLAFSAAAPAFKGWLADQDVRWNVISGAVDDRTPKERGVAPLLPKYNKNGFGGIAKDVQDKVLEIPKSRYSSVDLFLGGSKFFNRTYNDTNVPINEKVLGRLLENDKAPLDYDLAKHFAHLYIRDPVSTFEELLNQDNKTSSNHFENIQSTNWQTLRFKPPTQQATPDKKDSPGWRVEFRPFEVQLLDFENAAYSVLIYLIVDSILTFSDNINAYIHMSKVWENMKIAHHRDAILFEKFHWKKSFRNDTDVETEDYSISEIFHNPENGIFPQFVTPILCQKGFVTKDWKELKHSSKHERLYYYLKLISDRASGELPTTAKFFRNFVLQHPDYKHDSKISKSINYDLLSTCDRLTHLDDSKGELTSFLGAEIAEYVKKNKPSIESKC

SEQ ID NO: 46 - protein, 434 aa, Saccharomyces cerevisiae
MSNKFHCDVCSADCTNRVRVSCAICPEYDLCVPCFSQGSYTGKHRPYHDYRIIETNSYPILCPDWGADEELQLIKGAQTLGLGNWQDIADHIGSRGKEEVKEHYLKYYLESKYYPIPDITQNIHVPQDEFLEQRRHRIESFRERPLEPPRKPMASVPSCHEVQGFMPGRLEFETEFENEAEGPVKDMVFEPDDQPLDIELKFAILDIYNSRLTTRAEKKRLLFENHLMDYRKLQAIDKKRSKEAKELYNRIKPFARVMTAQDFEEFSKDILEELHCRARIQQLQEWRSNGLTTLEAGLKYERDKQARISSFEKFGASTAASLSEGNSRYRSNSAHRSNAEYSQNYSENGGRKKNMTISDIQHAPDYALLSNDEQQLCIQLKILPKPYLVLKEVMFRELLKTGGNLSKSACRELLNIDPIKANRIYDFFQSQNWM

SEQ ID NO: 47 - protein, 349 aa, Saccharomyces cerevisiae
MSQYIGKTISLISVTDNRYVGLLEDIDSEKGTVTLKEVRCFGTEGRKNWGPEEIYPNPTVYNSVKFNGSEVKDLSILDANINDIQPVVPQMMPPASQFPPQQAQSPPQAQAQAHVQTNPQVPKPESNVPAAVAGYGVYTPTSTETATASMNDKSTPQDTNVNSQSRERGKNGENEPKYQRNKNRSSNRPPQSNRNFKVDIPNEDFDFQSNNAKFTKGDSTDVEKEKELESAVHKQDESDEQFYNKKSSFFDTISTSTETNTNMRWQEEKMLNVDTFGQASARPRFHSRGLGRGRGNYRGNRGNRGRGGQRGNYQNRNNYQNDSGAYQNQNDSYSRPANQFSQPPSNVEF

SEQ ID NO: 48 - protein, 551 aa, Saccharomyces cerevisiae
MTYSTVSINTPPPYLTLACNEKLPTVLSIAGTDPSGGAGIEADVKTITAHRCYAMTCITALNAQTPVKVYSINNTPKEVVFQTLESNLKDMKCNVIKTGMLTAAAIEVLHEKLLQLGENRPKLVVDPVLVATSGSSLAGKDIVSLITEKVAPFADILTPNIPECYKLLGEERKVNGLQDIFQIAKDLAKITKCSNILVKGGHIPWNDEKEKYITDVLFLGAEQKFIIFKGNFVNTTHTHGTGCTLASAIASNLARGYSLPQSVYGGIEYVQNAVAIGCDVTKETVKDNGPINHVYAVEIPLEKMLSDECFTASDVIPKKPLKSAADKIPGGNFYEYLINHPKVKPHWDSYINHEFVKKVADGTLERKKFQFFIEQDYAYLVDYARVHCIAGSKAPCLEDMEKELVIVGGVRTEMGQHEKRLKEVFGVKDPDYFQKIKRGPALRAYSRYFNDVSRRGNWQELVASLTPCLMGYGEALTKMKGKVTAPEGSVYHEWCETYASSWYREAMDEGEKLLNHILETYPPEQLDTLVTIYAEVCELETNFWTAALEYE

SEQ ID NO: 49 - protein, 720 aa, Saccharomyces cerevisiae
MAKRVADAQIQRETYDSNESDDDVTPSTKVASSAVMNRRKIAMPKRRMAFKPFGSAKSDETKQASSFSFLNRADGTGEAQVDNSPTTESNSRLKALNLQFKAKVDDLVLGKPLADLRPLFTRYELYIKNILEAPVKSIENPTQTKGNDAKPAKVEDVQKSSDSSSEDEVKVEGPKFTIDAKPPISDSVFSFGPKKENRKKDESDSENDIEIKGPEFKFSGTVSSDVFKLNPSTDKNEKKTETNAKPFSFSSATSTTEQTKSKNPLSLTEATKTNVDNNSKAEASFTFGTKHAADSQNNKPSFVFGQAAAKPSLEKSSFTFGSTTIEKKNDENSTSNSKPEKSSDSNDSNPSFSFSIPSKNTPDASKPSFSFGVPNSSKNETSKPVFSFGAATPSAKEASQEDDNNNVEKPSSKPAFNLISNAGTEKEKESKKDSKPAFSFGISNGSESKDSDKPSLPSAVDGENDKKEATKPAFSFGINTNTTKTADTKAPTFTFGSSALADNKEDVKKPFSFGTSQPNNTPSFSFGKTTANLPANSSTSPAPSIPSTGFKFSLPFEQKGSQTTTNDSKEESTTEATGNESQDATKVDATPEESKPINLQNGEEDEVALFSQKAKLMTFNAETKSYDSRGVGEMKLLKKKDDPSKVRLLCRSDGMGNVLLNATVVDSFKYEPLAPGNDNLIKAPTVAADGKLVTYIVKFKQKEEGRSFTKAIEDAKKEMK

SEQ ID NO: 50 - protein, 232 aa, Saccharomyces cerevisiae
MWCYSHFLLIFVSFVTSFAHKLPANNSTTNGGTDGIAVPVIETTIDSGMYSENGTDLMTPEDLPDLLSDGIVLSFANTTETGSDSDSTLIDSEDLQRCIDIPDRSCSAQRGNLCSYSFWDIPFSFLNTVHDIFGMTNMGNCAVMAGNKGAFYYKYYPVEPNCNSTIHQKTIDDALQQAIKQLNGDFNNIYFFYVNRGGLWQGGMMVGTRVFTWFAGAKWAEHKGSIEAGFTS

SEQ ID NO: 51 - protein, 620 aa, Saccharomyces cerevisiae
MGRSKKRSRASSSRLNPLRKAGSNDNNKDTNVVNKKLQPLLQNLSSVVPNDRSIALSSISVLCEDAHMRQLLLKEKLVPIILNKLLNDSNSDIVVESFGLLRNLSLEEGYDVSIYLWRSDIWTSITSNFGRIVESLSALQAAEQQPQLKPAGKSKIESKRLLFDFADNLLSLVVALSNGSDDILNEILTESKINEIFQVISQLLKYGVEKLPINLFNTTLDLIYDLSSESFEFIDHVSNNELLSQFLNGLSPALHPQANELTKVLIEGIHCQFLDMKITYDQCNKMIHSVCHSINNIDPVQLVNDINNPVEIGPATSKDESSKVITKIKDYNAKRNESMIKLQSIEIAIDLITAIIEIVASKYESPESQEVAIPEELINTLTNFLPHVFMILKDTFTSRILIGWNNLIWLFVSLSLTELSGELLTTLWSYVTQLDSQDDLSIKIGRMGCIWALLKLIFPDGAFESENRALINVQMLNNSGFARGIIEEFQNNNDLELQQKCINVLSTYAMIQGQIDANKEIGQFFIQTLTQLNVRPEILIEMTNSLFQIYGDASYDYNEPIFVRGGFLSILKDQVVPNLRQQFKMVDKNKNPELKERCHDCFTTLDSFIHYKMNENSTNQ

SEQ ID NO: 52 - protein, 730 aa, Saccharomyces cerevisiae
MQDDPENSKLYDLLNSHLDVHGRSNEEPRQTGDSRSQSSGNTGENEEDIAFASGLNGGTFDSMLEALPDDLYFTDFVSPFTAAATTSVTTKTVKDTTPATNHMDDDIAMFDSLATTQPIDIAASNQQNGEIAQLWDFNVDQFNMTPSNSSGSATISAPNSFTSDIPQYNHGSLGNSVSKSSLFSYNSSTSNSNINQPSINNNSNTNAQSHHSFNIYKLQNNNSSSSAMNITNNNNSNNSNIQHPFLKKSDSIGLSSSNTTNSVRKNSLIKPMSSTSLANFKRAASVSSSISNMEPSGQNKKPLIQCFNCKTFKTPLWRRSPEGNTLCNACGLFQKLHGTMRPLSLKSDVIKKRISKKRAKQTDSNIAQNTPNASATSSTSVTTTNAKPIRSRKKSLQQNSLSRVIPEEIISDNIGNTNNILNVNRGGYNFNSVPSPVLMNSQSYNSSNANFNGASNANLNSNNLMRHNSNTVTPNFRRSSRRSSTSSNTSSSSKSSSRSVVPILPKPSPNSANSQQFNMNMNLMNTTNNISAGNSVASSPRIISSANFNSNSPLQQNLLSNSFQRQGMNIPRRKMSRNASYSSSFMAASLQQLHEQQQVDVNSNTNTNSNRQNWNSSNSVSTNSRSSNFVSQKPNFDIFNTPIDSPSVSRPSSRKSHTSLLSQQLQNSESNSFISNHKFNNRLSSDSTSPIKYEADVSTGGKISEDNSTKGSSKESSAIADELDWLKFGI

SEQ ID NO: 53 - protein, 456 aa, Saccharomyces cerevisiae
MQPKTFVHQLHAILLEPEVNKWIYWSPTDNTVFFLKPYDPNFSTHVLKRYFKHGNVNSFVRQLHMYGFHKLSHPSPDQSSANNGNVKELVEWKFTHPSGFFFKEANAGILNKIQRKSTGVGKDGKRKNILSPISVSYVDASRLNVLSQQSGPVSAREPSNMFMGSPVHYSTSQSPPHISIPQQQQSSGPYLISSLPPQQPTVNMMRRQSISARMMNSYDYPNQFSTQDSIVQPQQPQQVLSPQALSGPPMKKSGTLSSTDDLKTTSLPIVNYPMPYHPGAFAQQQQQQQQPLPTVPPYSSYSTPFPSMMNSLSNSASNSPALGVCNNNVTLPKKSNISERQALDNHIQTLKNSLSTITDLIEKHINSASQDENKTLTNDAMNKDLRTSLSLLQNSKEEIIQLESKWMSMQSVKTTALPLQETTNTSSTLTSLTSSIIPKSIPIITKGEVATKPASY

SEQ ID NO: 54 - protein, 200 aa, Saccharomyces cerevisiae
MGISRDSRHKRSATGAKRAQFRKKRKFELGRQPANTKIGAKRIHSVRTRGGNKKYRALRIETGNFSWASEGISKKTRIAGVVYHPSNNELVRTNTLTKAAIVQIDATPFRQWFEAHYGQTLGKKKNVKEEETVAKSKNAERKWAARAASAKIESSVESQFSAGRLYACISSRPGQSGRCDGYILEGEELAFYLRRLTAKK

SEQ ID NO: 55 - protein, 901 aa, Saccharomyces cerevisiae
MSHEWLISASETMRSIGNEEGLRDKGAVVANNDGEFNEGETNREEDSSTIFSFDFDEEIVMCIDFSGGKLGCSILDYHTKTLQAFDQDYVVNKTTISSHDLIDDADMSSNDISLLLGLLIMEANPTVCLVPARLEDWIFDYIKTKCDEINCRLELQPIKRFKKWDLLQSLQLRGHDNQTILNDILSNSKFTTTVTLGTVGCILANHEQLGEYNGSTASSNMVTGRLVQNAFEDVIHGIRYIDIRDRMVLDENTISALNIFPTAHKLGHDKMMRNGFFSVFELFNQVSSDYARRILKSWLINPLTNKKRIETRYSIIRTLLDKQNAIIFSDLTQSIKRCPDAFGFINQLRSGKSTLGTWSKVASFLEKGIAIFQLVSSLKLGSNEGNILHDIKNKVDISALKECLRKVETVIDFDTSRDTKTLTINTGVDNRLDECRNIYDHLEGILLDVARETQLFLLNTLPQEDCKTTKSLEKLVNAVYIPQLGYLVTISVLMEPLLDGIPNLQWEEIFRSSENIYFKNGRVLELDETYGDIYGAISDFEIEILFSLQEQILRRKTQLTAYNILLSELEILLSFAQVSAERNYAEPQLVEDECILEIINGRHALYETFLDNYIPNSTMIDGGLFSELSWCEQNKGRIIVVTGANASGKSVYLTQNGLIVYLAQIGCFVPAERARIGIADKILTRIRTQETVYKTQSSFLLDSQQMAKSLSLATEKSLILIDEYGKGTDILDGPSLFGSIMLNMSKSEKCPRIIACTHFHELFNENVLTENIKGIKHYCTDILISQKYNLLETAHVGEDHESEGITFLFKVKEGISKQSFGIYCAKVCGLSRDIVERAEELSRMINRGDDVVQQCGNLTEKEMREFQKNQEIVKKFLSWDLDLETTTTSENLRLKLKNFLR

SEQ ID NO: 56 - protein, 1432 aa, Saccharomyces cerevisiae
MSDIKQLLKEAKQELTNRDYEETIEISEKVLKLDPDNYFAHIFLGKALSSLPASNNVSSNRNLERATNHYVSAAKLVPDNLLAWKGLFLLFRTTEVVPDILSYDEYFDLCGQYADALLKQEQSQVELINDIKLLKKTHPDCQKAFYQHLKPGSLMAETIGRHLSTPQDALLNLIKILSNIETTEIGKTLSQNRLKLKASDPDYQIKLNSFSWEIIKNSEIDQLYNQLVNILADDQKRSEIENQWLEYRIKVLKSMPLDVKKDFFTKVKEMVEDMVLVNHQSLLAWQKYFEWTDYEDLDNMDAPLIIKYFKKFPKDPLAMILYSWLSSKLSKYDIKSLESANKPPEGHKKTEKETDIKDVDETNEDEVKDRVEDEVKDRVEDEVKDQDEEAKEDEEEDLDDIEIGLLEEEVVTVLTENIVKCKNNILAHRILCQYYLLTKEYEAALPYIKNGISLIAYNIKDLGVHLPLTKREFSLDLATVYTYVDAPKDHNAALKLYDNILSGDFSNIQAKMGKGIIFIERKNWKDAMTLLTQVHEQSPNNLEVLSELSWSKAHMGYMDEALAGLDTVIKGIKGMDLRSIDFRALNLWRQAKVYIMKHASINDAKQENVKCAFKLLIQSIKILDTFAPGFSTLGDIYCHYYKDHLRAFKCYFKAFDLDAGDYTAAKYITETYASKPNWQAASSIASRLIKGEKAKAELRSNNWPFRVVGIAHLEKQEESDSIEWFQSALRVDPNDVESWVGLGQAYHACGRIEASIKVFDKAIQLRPSHTFARYFKAISLCDVGEYLESLDILEKVCQEAATEESFQIGLVEVLMRCSLDLYSQGFLLKSVSIAKDTIERIKIIITELKCENQQVWIYLSQVLRLFIWIESKVDTLPVESLVSIFENSQFSGSEEIDSVDNIKIDTLLDSTTDDNVSIACKFLILASKYSVSDQKFTDIAGTVRASYWYNIGISELTAFITLKEPQYRDAAIFAFKKSIQLQSNTSETWIGLGIATMDINFRVSQHCFIKATALEPKATNTWFNLAMLGLKKKDTEFAQQVLNKLQSLAPQDSSPWLGMALILEEQGDIIGSSKLFAHSFILSNGRSKAAQFMYAKNVLENHINNGDDERDIETVEKLTTASIALEQFFKKSPDNQFALQCALLTLERLHHYENANELANRLIGILEKKFEKTQDERELFNFAIIKGQFARIHLGLGNFELSIENADLSQGIISESSDEKSMKTKISNHICLGLSYFFLNDFDQTLNQFQELLSISKDSKHLVVLIAKVLYDVGESDTKEIALQELTEYIATSGADLLVTLTIAAMSILDDKREDLSIILEELKALPLSKQIIDKHKDAPYLIEEITKRLYRNDTGKQVWQRSAYFFPNNLKVWERLDKNIQRRIASNGQNKVTAEEMSKLYCESKNLRSIQRGMFLCPWNVTAVKALNECF

SEQ ID NO: 57 - protein, 327 aa, Saccharomyces cerevisiae
MASSVPGPIDLPESRYDLSTYWGRIRHCAEISDPTMLLTTEKDLAHAREIISAYRHGELKETTPEFWRAKKQLDSTVHPDTGKTVLLPFRMSSNVLSNLVVTVGMLTPGLGTAGTVFWQWANQSLNVAVNSANANKSHPMSTSQLLTNYAAAVTASCGVALGLNNLVPRLKNISPHSKLILGRLVPFAAVVSAGIVNVFLMRGNEIRKGISVFDSNGDEVGKSKKAAFMAVGETALSRVINATPTMVIPPLILVRLQRGVLKGKSLGVQTLANLGLISVTMFSALPFALGIFPQRQAIHLNKLEPELHGKKDKDGKPIEKVYFNRGI

SEQ ID NO: 58 - protein, 1116 aa, Saccharomyces cerevisiae
MNQLTVSYGLISPDYCTSQDAHILPITKILYPDIPGKNYFLTSGRDGSIILHKNTQLSNEPETAATTIKNDAIRMQVHSDWASDLIHVNMKNSDPSAGDTFISVSHDFSIVLISVNAQLTTWDKKIIGDHDDYIKCIVPIHYEMSNDYELEEQEGGPDNVHDGINNGIVVDEQNNFLFVTGGLDRKIKLWCLSSGPEKMATLLHTFDNAQSNDTGSIYSMSPIIPKYSFDDNQTSRPFDFVAGDCNGDLIFYSCKYRKEVIRIQNAHRTNIKVVRTLDDSTRLISTSSDGVINVWDLNCRHDQTTGALQLPKKIGSWSWDSSIWCVQGTSLDKLYFGDSQGNVMRANLSSFEDAKLTRIFKPDHHHHHHHHHEHEEQNISTTDAKVKKYGGILDIALLPNEKLLFSFCTDSNLNVLDLTNNHFSVNEGGFALTRSSLLTNRRHVITENTKGQMQRWDIVSCELLNTFDSSEGSFDDIVMKYTSKEILSHWCTVSVKVGMLFVKINPKFLKTEIYGSALKDYQVVNNIEINSDERYNLGKIVINSLFNEFISYEVQKDKLLRKKIFSLKKKDLTNSLTLDTGYNSESKKNNKDKKRKSTFKISSTLSIGNTNSSGTPPNSAPATPVMAETIVLEEQPLLQSASDKAIDDSLELVQPLPASKKPYFRTQSSGSLLSRKFKSFRSTSGRATTGLNTPEEPKGILPDTPHVINDDSAFPQAINTTQQSKDATPESMLWNHPFKLEQKLSAISSQDLPSNNTHNKLRSSENSRANSTSTLEGNEKKKPEFMPDLLEQIQESYKQQYMNTSSLKYLTKRLPVTKIIKASSCPIIRVKSATLVLVHLWKEGSCGGRVLFSTLLPPSHVDNETVSGGKENSKPPDDEEVDLQAVDDDKLGKYDLIDGELGSRLNRRQIFEQLEENLPYWFAKALFRDIKTVEEQPKLNFLIMPWSSVGGSEAAGNENKKKFISASDTTESSGNDSSDSSLGNGNEAVSPSTQQQFHNMLKFGRPKTSEQELNPTDLPRISEANVKLVAPGMIRVKKIKLYVADRFETKTPEMKAKMEPSLWLDLLCRGQVLDNDMTLNTVRTLYWKSQGDIVLEYRRKVHNSPLVHEVNGNEGK

SEQ ID NO: 59 - protein, 397 aa, Saccharomyces cerevisiae
MSKVFIATANAGKAHDADIFSVSACNSFTVSCSGDGYLKVWDNKLLDNENPKDKSYSHFVHKSGLHHVDVLQAIERDAFELCLVATTSFSGDLLFYRITREDETKKVIFEKLDLLDSDMKKHSFWALKWGASNDRLLSHRLVATDVKGTTYIWKFHPFADESNSLTLNWSPTLELQGTVESPMTPSQFATSVDISERGLIATGFNNGTVQISELSTLRPLYNFESQHSMINNSNSIRSVKFSPQGSLLAIAHDSNSFGCITLYETEFGERIGSLSVPTHSSQASLGEFAHSSWVMSLSFNDSGETLCSAGWDGKLRFWDVKTKERITTLNMHCDDIEIEEDILAVDEHGDSLAEPGVFDVKFLKKGWRSGMGADLNESLCCVCLDRSIRWFREAGGK

SEQ ID NO: 60 - protein, 632 aa, Saccharomyces cerevisiae
MELNDPSIISSSQFSGELSDSDTAAATHKSQQAISNLFQKLAKKGREEKPIGSVESSTDSSNISVATSGNNKESNKKKNKKTAMLNFSSLTDPITNYKPMDLQYKTYAYSMNELYHLKPSLASASYEEDPLISELVRSLPKRKFWRLRMGPPDQKHANNHHFNGNNGGGSWKAGYKNGKNDERRMSRTKNMQGGKRRSQQDDEEKKIDQEMLEMDKNLQLGGDVGHSIADFEDWKAKMKELELKKLSKSKGISNSTAIAPRESASHETPTDLRPVIPRGPSSITDFLNLKRQDKKEESSQQTPGIPVGQPSLSKTSIEQVNELETNSDLGKSSSSRFSSFFNKSATSLPSLDNNNQVPSSNVSVVNNDGNSTPHQSGSRLMSFFKESRSSTPNAESQLLSASDKDNGKMQTLPQFQQQPQQMQPMAFTQHPPNNNAFFNGLLNKGKSETSTPPPPPPGLIAHQGPQFPVMGVPPNFPQRMMPPPPGLVQFQKDSKDVNKKEDRQLRQNKNPNGTRNSKGKQEETATPDLPQQQYMPPPPPPGFFPMHPNFPNGPMPPLPQGFPIPPNGMLPVTGQQPQPPYPNMMLQGNFPPNFQQGFGSNSPMPIPSIINANGKNVTNQLPPGLNSKKNIK

SEQ ID NO: 61 - protein, 406 aa, Saccharomyces cerevisiae
MGDELHNRLLHQNDGTKDAILYKIIESLVCSICHDYMFVPMMTPCGHNYCYGCLNTWFASNTQKELACPQCRSDITTIPALNTTLQQYLSFILEKLRDQNDESFKKLLTTKTKEENDYKNDKEKDTLFDKVFKNSALAVADDSDDGITRCSNCHWELDPDEVEDGNVCPHCNARIRNYAGGRDEFDEEEYSEGELDEIRESMRRRRENRFASTNPFANRDDVSSEDDDSSEEEPMREHIPLGRWARSHNRSIAVDAVDDEDDEEEDEEEEEEMDSDLKDFIEDDEDDEDEDGSRRNLVLSALKNRHVIITDDEEEEQRRHATEEEDRDSDFYEHNDDGFVSGDSLDEDQKEVTRIQSSSDSEDRSLSYSGSSDVKDNNDDNTEELDDPQPKRQKRFRVVLGDSDDE

SEQ ID NO: 62 - protein, 670 aa, Saccharomyces cerevisiae
MVEPDMQKKASGGSGGSEMDTLNATSNSSKQGVSNNKRNPVSKKKPGNKVSDGRDNAHNYHGEGRRKSSKQQRSRTPYKETSTRINDQDIDLSIQEEILGGNFKLRGRKTQVSINHLLNFQLPEVEREKSRSSSSKKSNRRRDEHVHLHGDTFVNVNYRLLVDDRFDYPEQNCNPNVPVDQEKILRVIVPKGQNCSICLSEEPVAPRMVTCGHIFCLSCLLNFFSIEETVKNKETGYSKKKKYKECPLCGSIIGPKRVKPVLYEDDFDVTRLNQKPEPGATVHLQLMCKPHGSLLPLPVALHLDPLKCGNFPPANLGSIKHYAHIMKCGVSYSLELYQKDIVAIQEQYEIDKAIYNDSGKFVKQSIENINDQISTLLAATTDLSPLSNDINNGLDNFHFDDDLLTKYDDSSAYFFYQTLVASSTKYFLSPLDVKILLTIFHYYSKFPESIETTVENIHYDTVVTEQLIRRYKYIGHLPIGTEIALLDLDWRKIPFLPKEIYEQFAHELKQRRRKFTMKKQKEDKEKKLYEKRLEQEHAEFYRKENGNSLKFEDSVQMATHYESIVSSSIPLNSLGISMLGPPTNSCSTPQKQAPSHTKRTIWGTSIAVTEDEKASKENKEFQDMLLQRIRQEDSSDVTDSTDSPPTSNGKRGRKKKGKVMLFSSNHQALG

SEQ ID NO: 63 - protein, 931 aa, Saccharomyces cerevisiae
MSTSTHSHKRKNSHLFPQRKSSNSSMDKPFFPNNDSVANTDPQSNENGHTINEIRPTEATIDVTDVPQTPFLQEQYSMRPRRESFQFNDIENQHHTHSFFSVNKFNRRWGEWSLPEKRSYVLVFTLIALSVLVLLVILIPSKLLPTKITRPKTSAGDSSLGKRSFSIENVLNGDFAIPEDTFHFIDPPQRLLGQDSDPGLYFTTKEIDGHTHFIAKQLFDETFEVNLGGNRFLYEGVEFTVSTVQINYKLDKLIFGTNLESEFRHSSKGFYWIKDLNTGNIEPILPPEKSDDNYELGLSKLSYAHFSPAYNYIYFVYENNLFLQQVNSGVAKKVTEDGSKDIFNAKPDWIYEEEVLASDQAIWWAPDDSKAVFARFNDTSVDDIRLNRYTNMNEAYLSDTKIKYPKPGFQNPQFDLFLVNLQNGIIYSINTGGQKDSILYNGKWISPDTFRFEITDRNSKILDVKVYDIPSSQMLTVRNTNSNLFNGWIEKTKDILSIPPKPELKRMDYGYIDIHADSRGFSHLFYYPTVFAKEPIQLTKGNWEVTGNGIVGYEYETDTIFFTANEIGVMSQHLYSISLTDSTTQNTFQSLQNPSDKYDFYDFELSSSARYAISKKLGPDTPIKVAGPLTRVLNVAEIHDDSILQLTKDEKFKEKIKNYDLPITSYKTMVLDDGVEINYIEIKPANLNPKKKYPILVNIYGGPGSQTFTTKSSLAFEQAVVSGLDVIVLQIEPRGTGGKGWSFRSWAREKLGYWEPRDITEVTKKFIQRNSQHIDESKIAIWGWSYGGFTSLKTVELDNGDTFKYAMAVAPVTNWTLYDSVYTERYMNQPSENHEGYFEVSTIQNFKSFESLKRLFIVHGTFDDNVHIQNTFRLVDQLNLLGLTNYDMHIFPDSDHSIRYHNAQRIVFQKLYYWLRDAFAGRFDNTELLHL

SEQ ID NO: 64 - protein, 1230 aa, Saccharomyces cerevisiae
MSSEDELGSIGTVFPGSPIDKSIGSILPQFDEEVETLLEDSFTWNIPDWNELTNPKYNSPRFRIGDFEWDILLFPQGNHNKGVAVYLEPHPEEKLDETTGEMVPVDPDWYCCAQFAIGISRPGNGDTINLINKSHHRFNALDTDWGFANLIDLNNLKHPSKGRPLSFLNEGTLNITAYVRILKDPTGVLWHNFLNYDSKKVTGYVGFRNQGATCYLNSLLQSYFFTKYFRKLVYEIPTEHESPNNSVPLALQRAFYQLQVSDIPLDTLELTRSFGWDTAESFTQHDVQELNRILMDRLENNMKGTSVEGKLNEIFVGKMKSYIKCINVDYESARVEDFWDLQLNVKNFKNLQESFDNYIEMELMNGENQYAAQDYGLQDAQKGVIFESFPPVLHLQLKRFEYDFNYDQMVKVNDKYEFPETIDLSPFVDKDVLKKTLDSENKDKNPYVYNLHGVLVHSGDISTGHYYTLIKPGVEDQWYRFDDERVWRVTKKQVFQENFGCDRLPDEKVRTMTRDEYQNYIIQRHTSAYMLVYIRQEQEEDLLRPVLESDVPKHVITRVREEIKERETKEKEIREAHLYVTLRLHSIKEFIHYEGFDYFAHDGFRLFAEELNDSGLQQINLKVLRTTKLSDIFASIKETMNIPQERDVKYWKMDYRRNSTLRLTQPINFESVNITLQEALKKEKKRTMQTQYGEEGVASTEEDDKALLETVSFLDLFIEEPYLELQFLNKLKEASLISKAQLDDELISTIRTNLPELTKGGIEPVFATDNKSNLLFVKSYDPHTQKLLGFGHFAVNQLQQLSDLSAIIEDSISSNEKLTFYEEVQPGTINEIYMKETIYDADIDTGDIVSFEVPGAVLPDTFPVYATIKDFYSYLRYRVKLKFSKFDGSSEEYGVSNEIPESFEFWISAYAPYDDLARMVSKYAHVKPEYLKIFALYSNGRFVLKSTSLLNDYLLKDFNCDQIPPFAFEVLSVPLKELERLRPIKLYWLKNSYIHYQCFEFEVANDYTESQFLEKVQHKIGFTDEEKENILLWTNTNFQFQGLLSDQNTFKDVSKNSLLFGRILPEESKLFKELNRLENVQTSSLEDFMDDENATDRPMDDEQDLGMAIEHSEDMKGRIVVVQQYFKDLENRHGISFLFNLIPDETFPKTKDRLHAKFGLGQKEFSKIKLSIGYSTEEGTVFRSLQGFSDEELDKVILYDIMSNLDYIYMDHPDRLRSHSSYDRPMIIKN

SEQ ID NO: 65 - protein, 712 aa, Saccharomyces cerevisiae
MLNSALLWKVWLRIDNSTDEVNQPIAVQFDEIDTVDDLKSRFFQKLSSTRWREINDNASIAIGLYAPKFDNQADNTSSNNTNDNSCRSKSNGAGSGANLSVNSNTKSSVSPTAGSFGLSKDLAKDRNVLQHPKPTQKRGALYDAFAAAPTVAATTNVDFPPNEAPMLSPQRPYSTSPKQFPATTKSPLLRFASVSPYPKFHSDNQIMASAGLTYVSPHNKNKYTRPLIRKGLNFTTESVNDCTYKIIFEPDELAINIYKELFGTMGSQPASQPLLIFSNVNLRQDVPPLDILNVVDYVPTNEEISQQKTQPTDHGAVGVFHLDDHISPGEQGLKQTIGDKADLKGKDGNSSPQEFKLITDEEQLRRASQELKDEEKDAESPWQAILLLPKGYKGGVDFRNKPVAHTDSSFNNEDTITHSELEVNTGSPSQESGSLNEAGIGITQPMSEVQRRKEDVTPASPILTSSQTPHYSNSLYNAPFAVSSPPDPLPNLFTTTSEKVFPKINVLIVEDNVINQAILGSFLRKHKISYKLAKNGQEAVNIWKEGGLHLIFMDLQLPVLSGIEAAKQIRDFEKQNGIGIQKSLNNSHSNLEKGTSKRFSQAPVIIVALTASNSQMDKRKALLSGCNDYLTKPVNLHWLSKKITEWGCMQALIDFDSWKQGESRMTDSVLVKSPQKPIAPSNPHSFKQATSMTPTHSPVRKNSNLSPTQIEL

SEQ ID NO: 66 - protein, 217 aa, Saccharomyces cerevisiae
MGVKQTPPVQVKVSDADSTNRRKSSSQEGNPQLVQLKAKSDKDKRKGSSDSTASIMGSSNALPTKNLTTPPALNPLTTSISRGNTAYERSVNGSRITMHSNLAPTETQDVSWSEIDTLDDVKKMAKEPIVNDGFPRDFESNLTQMRKSHAQLLRLMRERNQRLKYAKLRSPPHKDQHNSATNKDQEPDEVLHDPEIALDGEKYVSQVVDTIKDVHRC

SEQ ID NO: 67 - protein, 75 aa, Saccharomyces cerevisiae
MEAEKQSDIKGTIAFDTHGNVIESTGVGSQRIEDIGDLSKVTLDAEGFAQVQGDSLLVHLYKRNDITLAVYTSAQ

SEQ ID NO: 68 - protein, 542 aa, Saccharomyces cerevisiae
MSVQKEEYDIVEKAQLSVSAESLTSDSESISHNPFDDFHKAERWRKVYESSGYEGLSKFDPEFTWTKDEEKKLVRKMDLKIFLWVFIMFAFLDLIRKNIARAVSDNFIVDLKMNTNDYNLGQTVYLVIFLASELPGNLLSKRFGPERVIPVQIVLWSVICITQAGLKNRGQFIATRCLLGMVQGGFIPDNILYLSYYYTGAELTFRLSFFWCAIPLFQILGSLLASGIIEMRGIHNLAGWQYLFIIEGFLSLSVGVASFYLMRRGPTQTGESAFHKGKSLFTEYEEKIMVNRILRDDPSKGDMSNRQPVTFKEILYTLTEFDLWPLFIQGITAFISLQTVGSYLSLILKSLNYSTFLSNILAIPGQALLLINLPLAALLSRKLKEKSLCVGIANVWVLPFIVSLVALPTDTNPWIKYILLTGILGLPYTHSILAGWVSEISNSVRSRTVGTALYNMSAQVGAIIASNMYRNDDKPYYTRGNKILLGFTCFNICMAVATKFYYISRNKYKDRKWNSMTKEEQINYLDTTKDKGMKRLDYRFIH

SEQ ID NO: 69 - protein, 624 aa, Saccharomyces cerevisiae
MVVQKKLRAILTDEGVLIKSQSHHMFNKHGQLRSGDSLSLLSCLSCLDDGTLSSDGGSFDEDDSLELLPLNTTIPFNRILNAKYVNVGQKGFNNGKISSNPFQTENLSSSSENDDVENHSLSNDKAPVSESQSFPKKDKWDTKTNTVKVSPDDSQDNSPSLGIKDNQQLIELTFAVPKGHDVIPQKLTLLIDHVSRKSRANTGEENISSGTVEEILEKSYENSKRNRSILVIINPHGGKGTAKNLFLTKARPILVESGCKIEIAYTKYARHAIDIAKDLDISKYDTIACASGDGIPYEVINGLYRRPDRVDAFNKLAVTQLPCGSGNAMSISCHWTNNPSYAALCLVKSIETRIDLMCCSQPSYMNEWPRLSFLSQTYGVIAESDINTEFIRWMGPVRFNLGVAFNIIQGKKYPCEVFVKYAAKSKKELKVHFLENKDKNKGCLTFEPNPSPNSSPDLLSKNNINNSTKDELSPNFLNEDNFKLKYPMTEPVPRDWEKMDSELTDNLTIFYTGKMPYIAKDTKFFPAALPADGTIDLVITDARIPVTRMTPILLSLDKGSHVLEPEVIHSKILAYKIIPKVESGLFSVDGEKFPLEPLQVEIMPMLCKTLLRNGRYIDTEFESM

SEQ ID NO: 70 - protein, 1380 aa, Saccharomyces cerevisiae
MEPFAFGRGAPALCILTAAARINLDNFVPCCWALFRLSFFFPLDPAYIRNENKETRTSWISIEFFFFVKHCLSQHTFFSKTLAPKRNFRAKKLKDIGDTRIDRADKDFLLVPEPSMFVNGNQSNFAKPAGQGILPIPKKSRIIKTDKPRPFLCPTCTRGFVRQEHLKRHQHSHTREKPYLCIFCGRCFARRDLVLRHQQKLHAALVGTGDPRRMTPAPNSTSSFASKRRHSVAADDPTDLHIIKIAGNKETILPTPKNLAGKTSEELKEAVVALAKSNNVELPVSAPVMNDKREKTPPSKAGSLGFREFKFSTKGVPVHSASSDAVIDRANTPSSMHKTKRHASFSASSAMTYMSSSNSPHHSITNFELVEDAPHQVGFSTPQMTAKQLMESVSELDLPPLTLDEPPQAIKFNLNLFNNDPSGQQQQQQQQQQNSTSSTIVNSNNGSTVATPGVYLLSSGPSLTDLLTMNSAHAGAGGYMSSHHSPFDLGCFSHDKPTVSEFNLPSSFPNTIPSNSTTASNSYSNLANQTYRQMSNEQPLMSLSPKNPPTTVSDSSSTINFNPGTNNLLEPSMEPNDKDSNIDPAAIDDKWLSEFINNSDPKSTFKINFNHFNDIGFIYSPPSSRSSIPNKSPPNHSATSLNHEKASLSPRLNLSLNGSTDLPSTPQNQLKEPSYSDPISHSSHKRRRDSVMMDYDLSNFFSSRQLDISKVLNGTEQNNSHVNDDVLTLSFPGETDSNATQKQLPVLTPSDLLSPFSVPSVSQVLFTNELRSMMLADNNIDSGAFPTTSQLNDYVTYYKEEFHPFFSFIHLPSIIPNMDSYPLLLSISMVGALYGFHSTHAKVLANAASTQIRKSLKVSEKNPETTELWVIQTLVLLTFYCIFNKNTAVIKGMHGQLTTIIRLLKASRLNLPLESLCQPPIESDHIMEYENSPHMFSKIREQYNAPNQMNKNYQYFVLAQSRIRTCHAVLLISNLFSSLVGADCCFHSVDLKCGVPCYKEELYQCRNSDEWSDLLCQYKITLDSKFSLIELSNGNEAYENCLRFLSTGDSFFYGNARVSLSTCLSLLISIHEKILIERNNARISNNNTNSNNIELDDIEWKMTSRQRIDTMLKYWENLYLKNGGILTPTENSMSTINANPAMRLIIPVYLFAKMRRCLDLAHVIEKIWLKDWSNMNKALEEVCYDMGSLREATEYALNMVDAWTSFFTYIKQGKRRIFNTPVFATTCMFTAVLVISEYMKCVEDWARGYNANNPNSALLDFSDRVLWLKAERILRRLQMNLIPKECDVLKSYTDFLRWQDKDALDLSALNEEQAQRAMDPNTDINETIQLIVAASLSSKCLYLGVQILGDAPIWPIILSFAHGLQSRAIYSVTKKRNTRI

SEQ ID NO: 71 - protein, 799 aa, Saccharomyces cerevisiae [sequence omitted]

SEQ ID NO: 72 - protein, 393 aa, Saccharomyces cerevisiae
MLSRTIRTQGSFLRRSQLTITRSFSVTFNMQNAQKRSPTGIVLMNMGGPSKVEETYDFLYQLFADNDLIPISAKYQKTIAKYIAKFRTPKIEKQYREIGGGSPIRKWSEYQATEVCKILDKTCPETAPHKPYVAFRYAKPLTAETYKQMLKDGVKKAVAFSQYPHFSYSTTGSSINELWRQIKALDSERSISWSVIDRWPTNEGLIKAFSENITKKLQEFPQPVRDKVVLLFSAHSLPMDVVNTGDAYPAEVAATVYNIMQKLKFKNPYRLVWQSQVGPKPWLGAQTAEIAEFLGPKVDGLMFIPIAFTSDHIETLHEIDLGVIGESEYKDKFKRCESLNGNQTFIEGMADLVKSHLQSNQLYSNQLPLDFALGKSNDPVKDLSLVFGNHEST

SEQ ID NO: 73 - protein, 299 aa, Saccharomyces cerevisiae
MKIQAGQLGLDDNDVPGPLPDTDSKPSSQSQNDTPMFKLGNFESPVLKELSRRTVNKEMETQRIMTNVIAFAFWNLLVKFIKFFWNNTHVGRQFCNRLSRIHLYMLTFHTVKKANIIYHTTFSWLNAELLDYLFHLLVSLNILFSLWKLLSTVKVSDLNLTERQKKLLGVDMQSSVDTGLQPQHPHYVSTSKTSQMAQNKTHIPQTNLKNHPAYLFKGLETPLKARQREMAEEQTKLQSQSLHTKNVFGTLQRHSGISSTLVSANNDNNSPHTPVTRKGYIPSSKYAYMMNSQSPRGKI

SEQ ID NO: 74 - DNA, 39 bp, synthetic construct
  source 1..39: mol_type = other DNA
atccagtatgatttttaataataactggaagaaaatgat

SEQ ID NO: 75 - DNA, 42 bp, synthetic construct
  source 1..42: mol_type = other DNA
tacctagccaagttgtaataataagcggaacgttatgaagaa

SEQ ID NO: 76 - DNA, 39 bp, synthetic construct
  source 1..39: mol_type = other DNA
cttatggacgtcgattaataataatttttggacgacgga

SEQ ID NO: 77 - DNA, 48 bp, synthetic construct
  source 1..48: mol_type = other DNA
actgccacaaattcataataataatgtttcagcaatatttctatctta

SEQ ID NO: 78 - DNA, 42 bp, synthetic construct
  source 1..42: mol_type = other DNA
gggattgctgaagaaggctaataataatgttctaaagtggag

SEQ ID NO: 79 - DNA, 39 bp, synthetic construct
  source 1..39: mol_type = other DNA
tacctagctaaattataataataagccgaacgttatgaa

SEQ ID NO: 80 - DNA, 39 bp, synthetic construct
  source 1..39: mol_type = other DNA
aacgacgtcgaagactaataataagtcgttttgccacca

SEQ ID NO: 81 - DNA, 39 bp, synthetic construct
  source 1..39: mol_type = other DNA
aaagggtttctggaataataataaaaaccgtatgatttg

SEQ ID NO: 82 - DNA, 39 bp, synthetic construct
  source 1..39: mol_type = other DNA
tctttgaacgttacttaataataatccacatctgagatt

SEQ ID NO: 83 - DNA, 39 bp, synthetic construct
  source 1..39: mol_type = other DNA
gcattaagtgctctttaataataaccaggaaacagtcat

SEQ ID NO: 84 - DNA, 48 bp, synthetic construct
  source 1..48: mol_type = other DNA
gcacatcaaggcgggcgtagccagtaataataagaattgtattcgtgc

SEQ ID NO: 85 - DNA, 39 bp, synthetic construct
  source 1..39: mol_type = other DNA
gctccattcggtgcttaataataaaagtctaacaagact

SEQ ID NO: 86 - DNA, 39 bp, synthetic construct
  source 1..39: mol_type = other DNA
aagctgggcagttcgtaataataagacgagagcacaaaa

SEQ ID NO: 87 - DNA, 39 bp, synthetic construct
  source 1..39: mol_type = other DNA
aatctcagtaataactaataataaaaacaggtttactac

SEQ ID NO: 88 - DNA, 60 bp, synthetic construct
  source 1..60: mol_type = other DNA
acgtcgcagcgacagctgagacagaatgggagtccgtaataataatcacgttcatcacaa

SEQ ID NO: 89 - DNA, 39 bp, synthetic construct
  source 1..39: mol_type = other DNA
gataacataaccaattaataataacttccgtctggaccc

SEQ ID NO: 90 - DNA, 39 bp, synthetic construct
  source 1..39: mol_type = other DNA
tttatcatcggtggttaataataaattggttttaatttg

SEQ ID NO: 91 - DNA, 57 bp, synthetic construct
  source 1..57: mol_type = other DNA
actggaaaaatggcttaataataaaggcagattctggcgtctccactgtttttcgca

SEQ ID NO: 92 - DNA, 39 bp, synthetic construct
  source 1..39: mol_type = other DNA
attgaatcagaatcttaataataacagccgacgctggat

SEQ ID NO: 93 - DNA, 39 bp, synthetic construct
  source 1..39: mol_type = other DNA
gccacggagcagacttaataataagtttttcagaagcta

SEQ ID NO: 94 - DNA, 39 bp, synthetic construct
  source 1..39: mol_type = other DNA
aaaataaaacatgcgtaataataaagaaaacatcattcc

SEQ ID NO: 95 - DNA, 39 bp, synthetic construct
  source 1..39: mol_type = other DNA
tttttgctagcaatctaataataattcgaaatacttccg

SEQ ID NO: 96 - DNA, 39 bp, synthetic construct
  source 1..39: mol_type = other DNA
ctctttcatgatcgataataataagattactctcatact

SEQ ID NO: 97 - DNA, 39 bp, synthetic construct
  source 1..39: mol_type = other DNA
gcggccaacgagccataataataagactgtacctgtaaa

SEQ ID NO: 98 - DNA, 39 bp, synthetic construct
  source 1..39: mol_type = other DNA
tcgaccgtacagccataataataaagaggatcttaccca

SEQ ID NO: 99 - DNA, 39 bp, synthetic construct
  source 1..39: mol_type = other DNA
cttttcgaaagaggttaataataatcctatgccattgag

SEQ ID NO: 100 - DNA, 33 bp, synthetic construct
  source 1..33: mol_type = other DNA
aaagcacagcaagccgtgactgcaacagcagaa

SEQ ID NO: 101 - DNA, 40 bp, synthetic construct
  source 1..40: mol_type = other DNA
ttggttgtattgatcggttttaattggttcatcgtggttc

SEQ ID NO: 102 - DNA, 31 bp, synthetic construct
  source 1..31: mol_type = other DNA
cattgctttctggctcttactatcatttgga

SEQ ID NO: 103 - DNA, 45 bp, synthetic construct
  source 1..45: mol_type = other DNA
ctccattgctttctgaagggaatcaaggttttactatcatttgga

SEQ ID NO: 104 - DNA, 46 bp, synthetic construct
  source 1..46: mol_type = other DNA
tctccattgctttctaaaggggatcaaggttttactatcatttgga

SEQ ID NO: 105 - DNA, 35 bp, synthetic construct
  source 1..35: mol_type = other DNA
caccaacttgcggaacatccagtggaatcccgttc

SEQ ID NO: 106 - DNA, 39 bp, synthetic construct
  source 1..39: mol_type = other DNA
ataggtgatcatggcagctgttgcgctgaaatggatgtg

SEQ ID NO: 107 - DNA, 36 bp, synthetic construct
  source 1..36: mol_type = other DNA
gataccgatgacttctcacagcatggaggcctggca

SEQ ID NO: 108 - DNA, 45 bp, synthetic construct
  source 1..45: mol_type = other DNA
acttgcgatccagacgggtgtgacttcaacccataccgtatggga

SEQ ID NO: 109 - DNA, 36 bp, synthetic construct
  source 1..36: mol_type = other DNA
cacgacgttaatggttacaccaattgctacactgga

SEQ ID NO: 110 - DNA, 42 bp, synthetic construct
  source 1..42: mol_type = other DNA
cattgctttctggctacacccgtacacttactatcatttgga

SEQ ID NO: 111 - DNA, 46 bp, synthetic construct
  source 1..46: mol_type = other DNA
cattgctttctggctcggagtaaccgcgccgttactatcatttgga

SEQ ID NO: 112 - DNA, 41 bp, synthetic construct
  source 1..41: mol_type = other DNA
gttcatcgtggttcacggggttttcttttttctccattgct

SEQ ID NO: 113 - DNA, 38 bp, synthetic construct
  source 1..38: mol_type = other DNA
gttcatcgtggttcatccgcgggtttttctccattgct

SEQ ID NO: 114 - DNA, 40 bp, synthetic construct
  source 1..40: mol_type = other DNA
gttcatcgtggttcacgcggttttttttttctccattgct

SEQ ID NO: 115 - DNA, 38 bp, synthetic construct
  source 1..38: mol_type = other DNA
gtttctttggttgtatgacgtcaatttggttcatcgtg

SEQ ID NO: 116 - DNA, 42 bp, synthetic construct
  source 1..42: mol_type = other DNA
gttcatcgtggttcaacacccgtacactttttctccattgct

SEQ ID NO: 117 - DNA, 41 bp, synthetic construct
  source 1..41: mol_type = other DNA
gtttctttggttgtaattttgcggggatttggttcatcgtg

SEQ ID NO: 118 - DNA, 39 bp, synthetic construct
  source 1..39: mol_type = other DNA
gttcatcgtggttcacggatctaatttttctccattgct

SEQ ID NO: 119 - DNA, 36 bp, synthetic construct
  source 1..36: mol_type = other DNA
agtggcaccaagcatgaggcgaaaaaaaaagcatta

SEQ ID NO: 120 - DNA, 36 bp, synthetic construct
  source 1..36: mol_type = other DNA
gttcatcgtggttcacattcttttttctccattgct

SEQ ID NO: 121 - DNA, 37 bp, synthetic construct
  source 1..37: mol_type = other DNA
gttcatcgtggttcatgtttcatttttctccattgct

SEQ ID NO: 122 - DNA, 36 bp, synthetic construct
  source 1..36: mol_type = other DNA
cattgctttctggctccggggttactatcatttgga

SEQ ID NO: 123 - DNA, 36 bp, synthetic construct
  source 1..36: mol_type = other DNA
cattgctttctggctccccacttactatcatttgga

SEQ ID NO: 124 - DNA, 39 bp, synthetic construct
  source 1..39: mol_type = other DNA
gttcatcgtggttcaaccgctttttttttctccattgct

SEQ ID NO: 125 - DNA, 39 bp, synthetic construct
  source 1..39: mol_type = other DNA
ttggttcatcgtggtgaatgactctttttctccattgct

SEQ ID NO: 126 - DNA, 35 bp, synthetic construct
  source 1..35: mol_type = other DNA
cattgctttctggctaggggttactatcatttgga

SEQ ID NO: 127 - DNA, 47 bp, synthetic construct
  source 1..47: mol_type = other DNA
gttcatcgtggttcacgccacctatcattttttttttctccattgct

SEQ ID NO: 128 - DNA, 36 bp, synthetic construct
  source 1..36: mol_type = other DNA
gttcatcgtggttcaccccactttttctccattgct

SEQ ID NO: 129 - DNA, 31 bp, synthetic construct
  source 1..31: mol_type = other DNA
agagtacgaacgccagttagcctggtctcag

SEQ ID NO: 130 - DNA, 75 bp, synthetic construct
  source 1..75: mol_type = other DNA
ttcataatgttctccaagggacagtcgaacacacatcacattacacgatcgtacagcaatccattatttctgcaa

SEQ ID NO: 131 - DNA, 31 bp, synthetic construct
  source 1..31: mol_type = other DNA
accgactgccgtagtggtattttgtggaacc

SEQ ID NO: 132 - DNA, 31 bp, synthetic construct
  source 1..31: mol_type = other DNA
gagtcaaattcattcattgttcatgaacttt

SEQ ID NO: 133 - DNA, 31 bp, synthetic construct
  source 1..31: mol_type = other DNA
ctgcacacaacgggtaaagtgaagaagagtg

SEQ ID NO: 134 - DNA, 31 bp, synthetic construct
  source 1..31: mol_type = other DNA
ctgcctccactaccgtacgacagtaccttcc

SEQ ID NO: 135 - DNA, 57 bp, synthetic construct
  source 1..57: mol_type = other DNA
atgggttccagaagataataataaaggacaacaattttcagccctgagggacgtcta

SEQ ID NO: 136 - DNA, 51 bp, synthetic construct
  source 1..51: mol_type = other DNA
catcagattgaagagtaataataagatggcgcgaccacggatcccgaagtt

SEQ ID NO: 137 - DNA, 39 bp, synthetic construct
  source 1..39: mol_type = other DNA
atgacgatgcgaacataataataaaataagagttctaat

SEQ ID NO: 138 - DNA, 39 bp, synthetic construct
  source 1..39: mol_type = other DNA
atgagcatttgtccataataataacaagtatttcagaat

SEQ ID NO: 139 - DNA, 42 bp, synthetic construct
  source 1..42: mol_type = other DNA
atggcgaagattatgtaataataatctaagcagcctgtttac

SEQ ID NO: 140 - DNA, 39 bp, synthetic construct
  source 1..39: mol_type = other DNA
atgtcctcgtcttcgtaataataatacagcaaaaacaat

SEQ ID NO: 141 - DNA, 39 bp, synthetic construct
  source 1..39: mol_type = other DNA
atgaagtacgttgtttaataataaggtgtcatttcgggt

SEQ ID NO: 142 - DNA, 69 bp, synthetic construct
  source 1..69: mol_type = other DNA
gactctacccaatagtatataactgtctagaaataaagagtatcatctttcaaaatgacgtatcaatat

SEQ ID NO: 143 - DNA, 69 bp, synthetic construct
  source 1..69: mol_type = other DNA
gtcccttctacataatatataactgtctagaaataaagagtatcatctttcaaatacctctctgttctt

SEQ ID NO: 144 - DNA, 69 bp, synthetic construct
  source 1..69: mol_type = other DNA
acggttcccttttaatatataactgtctagaaataaagagtatcatctttcaaaaaataaatgttaaat

SEQ ID NO: 145 - DNA, 69 bp, synthetic construct
  source 1..69: mol_type = other DNA
tcgataactaaataatatataactgtctagaaataaagagtatcatctttcaaagtaactaccaataca

SEQ ID NO: 146 - DNA, 69 bp, synthetic construct
  source 1..69: mol_type = other DNA
tttatctcaagatagtatataactgtctagaaataaagagtatcatctttcaaaatcgtcatattcttt

SEQ ID NO: 147 - DNA, 69 bp, synthetic construct
  source 1..69: mol_type = other DNA
gtgccgtttaaatgatatataactgtctagaaataaagagtatcatctttcaaaagcctagattacgtt

SEQ ID NO: 148 - DNA, 78 bp, synthetic construct
  source 1..78: mol_type = other DNA
tctaacttgccatctcttgcgtgatatataactgtctagaaataaagagtatcatctttcaaaacagtgttctacttt

SEQ ID NO: 149 - DNA, 69 bp, synthetic construct
  source 1..69: mol_type = other DNA
gaaagcaaatgttaatatataactgtctagaaataaagagtatcatctttcaaacgatttgggtgacac

SEQ ID NO: 150 - DNA, 39 bp, synthetic construct
  source 1..39: mol_type = other DNA
tgttcagctgattgctaataataagtgagggtttcatgt

SEQ ID NO: 151 - DNA, 39 bp, synthetic construct
  source 1..39: mol_type = other DNA
tctttaatctctgtgtaataataaagatatgtggggctg

SEQ ID NO: 152 - DNA, 39 bp, synthetic construct
  source 1..39: mol_type = other DNA
acgcctccaccatattaataataagcctgcaacgaaaag

SEQ ID NO: 153 - DNA, 54 bp, synthetic construct
  source 1..54: mol_type = other DNA
gttgccgatgcgcaaattcaacgtgaaacgtaataataaaacgagtctgacgat

SEQ ID NO: 154 - DNA, 39 bp, synthetic construct
  source 1..39: mol_type = other DNA
atttttgtttccttctaataataatttgcacataagctc

SEQ ID NO: 155 - DNA, 39 bp, synthetic construct
  source 1..39: mol_type = other DNA
gcatcttcgtctcgataataataactacgcaaagctggg

SEQ ID NO: 156 - DNA, 60 bp, synthetic construct
  source 1..60: mol_type = other DNA
ctgtacgacctgctgtaataataactggacgtgcatggtcgttctaatgaagagccgaga

SEQ ID NO: 157 - DNA, 39 bp, synthetic construct
  source 1..39: mol_type = other DNA
cttcatgcaattctttaataataagaagtaaataaatgg

SEQ ID NO: 158 - DNA, 39 bp, synthetic construct
  source 1..39: mol_type = other DNA
aaaagatcagccacttaataataacgtgctcaattcaga

SEQ ID NO: 159 - DNA, 48 bp, synthetic construct
  source 1..48: mol_type = other DNA
tcggaaacaatgagataataataaaacgaagagggtttgcgggataaa

SEQ ID NO: 160 - DNA, 48 bp, synthetic construct
  source 1..48: mol_type = other DNA
gaagccaaacaagaataataataacgcgattacgaagagaccatcgaa

SEQ ID NO: 161 - DNA, 39 bp, synthetic construct
  source 1..39: mol_type = other DNA
gatttgcccgaatcctaataataattatccacgtattgg

SEQ ID NO: 162 - DNA, 39 bp, synthetic construct
  source 1..39: mol_type = other DNA
ctaatttccccagattaataataatcccaagatgcgcac

SEQ ID NO: 163 - DNA, 54 bp, synthetic construct
  source 1..54: mol_type = other DNA
tttattgccacagcaaacgcgggcaaggcttaataataagatattttctcggtt

SEQ ID NO: 164 - DNA, 39 bp, synthetic construct
  source 1..39: mol_type = other DNA
tcaagcagccagttttaataataattgtctgacagcgat

SEQ ID NO: 165 - DNA, 39 bp, synthetic construct
  source 1..39: mol_type = other DNA
cttcaccaaaacgattaataataagacgccatactttat

SEQ ID NO: 166 - DNA, 39 bp, synthetic construct
  source 1..39: mol_type = other DNA
gcaagtggtggcagttaataataagagatggatacttta

SEQ ID NO: 167 - DNA, 39 bp, synthetic construct
  source 1..39: mol_type = other DNA
aggaagaatagtcattaataataacaaaggaaaagttca

SEQ ID NO: 168 - DNA, 39 bp, synthetic construct
  source 1..39: mol_type = other DNA
atcggtactgtgttttaataataacccatagataagagc

SEQ ID NO: 169 - DNA, 39 bp, synthetic construct
  source 1..39: mol_type = other DNA
gtttggctacgaatataataataaactgatgaagtaaac

SEQ ID NO: 170 - DNA, 39 bp, synthetic construct
  source 1..39: mol_type = other DNA
caggtgaaagtaagctaataataatccaccaacagaagg

SEQ ID NO: 171 - DNA, 39 bp, synthetic construct
  source 1..39: mol_type = other DNA
aagggaaccattgcttaataataacacggtaacgttata

SEQ ID NO: 172 - DNA, 39 bp, synthetic construct
  source 1..39: mol_type = other DNA
attgtagaaaaggcctaataataagtgtccgcagaaagc

SEQ ID NO: 173 - DNA, 39 bp, synthetic construct
  source 1..39: mol_type = other DNA
atcttgaccgatgaataataataaatcaaatcgcaatca

SEQ ID NO: 174 - DNA, 39 bp, synthetic construct
  source 1..39: mol_type = other DNA
gcgcctgcattatgctaataataagcggccgctcgaata

SEQ ID NO: 175 - DNA, 45 bp, synthetic construct
  source 1..45: mol_type = other DNA
ttgaagtcagacactgacgtgtaataataaaatgcgtcaacagta

SEQ ID NO: 176 - DNA, 63 bp, synthetic construct
  source 1..63: mol_type = other DNA
ctttccagaacaatccgcacccagggctccttcctaagataataataactgaccattacaaga

SEQ ID NO: 177 - DNA, 45 bp, synthetic construct
  source 1..45: mol_type = other DNA
ttggacgataatgactaataataaccgctgccggacacagacagc

SEQ ID NO: 178 - DNA, 39 bp, synthetic construct; identical to SEQ ID NO: 74
  source 1..39: mol_type = other DNA

SEQ ID NO: 179 - DNA, 42 bp, synthetic construct
  source 1..42: mol_type = other DNA
tacctagccaagttgtaataataagccgaacgttatgaagaa

SEQ ID NO: 180 - DNA, 39 bp, synthetic construct; identical to SEQ ID NO: 76
  source 1..39: mol_type = other DNA

SEQ ID NO: 181 - DNA, 48 bp, synthetic construct
  source 1..48: mol_type = other DNA
actgccacaaattcataataataatgcttttctaatatttctatctta

SEQ ID NO: 182 - DNA, 42 bp, synthetic construct
  source 1..42: mol_type = other DNA
gggattgctgaagaaggataataataatgttctaaagtggag

SEQ ID NO: 183 - DNA, 39 bp, synthetic construct; identical to SEQ ID NO: 79
  source 1..39: mol_type = other DNA

SEQ ID NO: 184 - DNA, 39 bp, synthetic construct; identical to SEQ ID NO: 80
  source 1..39: mol_type = other DNA

SEQ ID NO: 185 - DNA, 39 bp, synthetic construct; identical to SEQ ID NO: 81
  source 1..39: mol_type = other DNA

SEQ ID NO: 186 - DNA, 39 bp, synthetic construct; identical to SEQ ID NO: 82
  source 1..39: mol_type = other DNA

SEQ ID NO: 187 - DNA, 39 bp, synthetic construct; identical to SEQ ID NO: 83
  source 1..39: mol_type = other DNA

SEQ ID NO: 188 - DNA, 48 bp, synthetic construct
  source 1..48: mol_type = other DNA
gcacatcaaggcgggagatcacaataataataagaattgtattcgtgc

SEQ ID NO: 189 - DNA, 39 bp, synthetic construct; identical to SEQ ID NO: 85
  source 1..39: mol_type = other DNA

SEQ ID NO: 190 - DNA, 39 bp, synthetic construct; identical to SEQ ID NO: 86
  source 1..39: mol_type = other DNA

SEQ ID NO: 191 - DNA, 39 bp, synthetic construct; identical to SEQ ID NO: 87
  source 1..39: mol_type = other DNA

SEQ ID NO: 192 - DNA, 60 bp, synthetic construct
  source 1..60: mol_type = other DNA
acgtcgcagcgacagcttcgacaaaatgggagtccgtaataataatcacgttcatcacaa

SEQ ID NO: 193 - DNA, 39 bp, synthetic construct; identical to SEQ ID NO: 89
  source 1..39: mol_type = other DNA

SEQ ID NO: 194 - DNA, 39 bp, synthetic construct; identical to SEQ ID NO: 90
  source 1..39: mol_type = other DNA

SEQ ID NO: 195 - DNA, 57 bp, synthetic construct
  source 1..57: mol_type = other DNA
actggaaaaatggcttaataataaaggcagattttggcaagcccactgtttttcgca

SEQ ID NO: 196 - DNA, 39 bp, synthetic construct; identical to SEQ ID NO: 92
  source 1..39: mol_type = other DNA

SEQ ID NO: 197 - DNA, 39 bp, synthetic construct; identical to SEQ ID NO: 93
  source 1..39: mol_type = other DNA

SEQ ID NO: 198 - DNA, 39 bp, synthetic construct; identical to SEQ ID NO: 94
  source 1..39: mol_type = other DNA

SEQ ID NO: 199 - DNA, 39 bp, synthetic construct; identical to SEQ ID NO: 95
  source 1..39: mol_type = other DNA

SEQ ID NO: 200 - DNA, 39 bp, synthetic construct; identical to SEQ ID NO: 96
  source 1..39: mol_type = other DNA

SEQ ID NO: 201 - DNA, 39 bp, synthetic construct; identical to SEQ ID NO: 97
  source 1..39: mol_type = other DNA

SEQ ID NO: 202 - DNA, 39 bp, synthetic construct; identical to SEQ ID NO: 98
  source 1..39: mol_type = other DNA

SEQ ID NO: 203 - DNA, 39 bp, synthetic construct; identical to SEQ ID NO: 99
  source 1..39: mol_type = other DNA

SEQ ID NO: 204 - DNA, 33 bp, synthetic construct; identical to SEQ ID NO: 100
  source 1..33: mol_type = other DNA

SEQ ID NO: 205 - DNA, 40 bp, synthetic construct
  source 1..40: mol_type = other DNA
ttggttgtattgatcatttttaattggttcatcgtggttc

SEQ ID NO: 206 - DNA, 31 bp, synthetic construct
  source 1..31: mol_type = other DNA
cattgctttctggctttgatcttactatcat

SEQ ID NO: 207 - DNA, 45 bp, synthetic construct
  source 1..45: mol_type = other DNA
ctccattgctttctggctttgatcaaggttttactatcatttgga

SEQ ID NO: 208 - DNA, 46 bp, synthetic construct
  source 1..46: mol_type = other DNA
tctccattgctttctggctttgatcaaggttttactatcatttgga

SEQ ID NO: 209 - DNA, 35 bp, synthetic construct; identical to SEQ ID NO: 105
  source 1..35: mol_type = other DNA

SEQ ID NO: 210 - DNA, 39 bp, synthetic construct
  source 1..39: mol_type = other DNA
ataggtgatcatggctcctgttgcgctgaaatggatgtg

SEQ ID NO: 211 - DNA, 36 bp, synthetic construct
  source 1..36: mol_type = other DNA
gataccgatgacttctcacaacatggaggcctggca

SEQ ID NO: 212 - DNA, 45 bp, synthetic construct
  source 1..45: mol_type = other DNA
acttgcgatccagacgggtgtgattttaatccataccgtatggga

SEQ ID NO: 213 - DNA, 36 bp, synthetic construct
  source 1..36: mol_type = other DNA
cacgacgttaatggttacacaaattgctacactgga

SEQ ID NO: 214 - DNA, 42 bp, synthetic construct; identical to SEQ ID NO: 110
  source 1..42: mol_type = other DNA

SEQ ID NO: 215 - DNA, 46 bp, synthetic construct; identical to SEQ ID NO: 111
  source 1..46: mol_type = other DNA

SEQ ID NO: 216 - DNA, 41 bp, synthetic construct; identical to SEQ ID NO: 112
  source 1..41: mol_type = other DNA

SEQ ID NO: 217 - DNA, 38 bp, synthetic construct; identical to SEQ ID NO: 113
  source 1..38: mol_type = other DNA

SEQ ID NO: 218 - DNA, 40 bp, synthetic construct; identical to SEQ ID NO: 114
  source 1..40: mol_type = other DNA

SEQ ID NO: 219 - DNA, 38 bp, synthetic construct; identical to SEQ ID NO: 115
  source 1..38: mol_type = other DNA

SEQ ID NO: 220 - DNA, 42 bp, synthetic construct; identical to SEQ ID NO: 116
  source 1..42: mol_type = other DNA

SEQ ID NO: 221 - DNA, 41 bp, synthetic construct; identical to SEQ ID NO: 117
  source 1..41: mol_type = other DNA

SEQ ID NO: 222 - DNA, 39 bp, synthetic construct; identical to SEQ ID NO: 118
  source 1..39: mol_type = other DNA

SEQ ID NO: 223 - DNA, 36 bp, synthetic construct; identical to SEQ ID NO: 119
  source 1..36: mol_type = other DNA

SEQ ID NO: 224 - DNA, 36 bp, synthetic construct
  source 1..36: mol_type = other DNA
gttcatcgtggttcacattctttttttctccattgc

SEQ ID NO: 225 - DNA, 37 bp, synthetic construct; identical to SEQ ID NO: 121
  source 1..37: mol_type = other DNA

SEQ ID NO: 226 - DNA, 36 bp, synthetic construct; identical to SEQ ID NO: 122
  source 1..36: mol_type = other DNA

SEQ ID NO: 227 - DNA, 36 bp, synthetic construct; identical to SEQ ID NO: 123
  source 1..36: mol_type = other DNA

SEQ ID NO: 228 - DNA, 39 bp, synthetic construct; identical to SEQ ID NO: 124
  source 1..39: mol_type = other DNA

SEQ ID NO: 229 - DNA, 39 bp, synthetic construct
  source 1..39: mol_type = other DNA
ttggttcatcgtggttcatgactcttttttctccattgc

SEQ ID NO: 230 - DNA, 35 bp, synthetic construct
  source 1..35: mol_type = other DNA
cattgctttctggctaggggcttactatcatttgg

SEQ ID NO: 231 - DNA, 47 bp, synthetic construct; identical to SEQ ID NO: 127
  source 1..47: mol_type = other DNA

SEQ ID NO: 232 - DNA, 36 bp, synthetic construct
  source 1..36: mol_type = other DNA
gttcatcgtggttcaccccacttttttctccattgc

SEQ ID NO: 233 - DNA, 31 bp, synthetic construct
  source 1..31: mol_type = other DNA
aggtatggctgcgcagttagcctggtctcag

SEQ ID NO: 234 - DNA, 75 bp, synthetic construct
  source 1..75: mol_type = other DNA
ttcataatgttctccgctttccagtcgaacacacatcacattacacgatcgtacagcaatgcttcagtagggcgg

SEQ ID NO: 235 - DNA, 31 bp, synthetic construct
  source 1..31: mol_type = other DNA
accgactgccgtagtacaaacgtctaaatcc

SEQ ID NO: 236 - DNA, 31 bp, synthetic construct
  source 1..31: mol_type = other DNA
tgtgcactcgataaaattgttcatgaacttt

SEQ ID NO: 237 - DNA, 31 bp, synthetic construct
  source 1..31: mol_type = other DNA
acgatgttagcagcgaaagtgaagaagagtg

SEQ ID NO: 238 - DNA, 31 bp, synthetic construct
  source 1..31: mol_type = other DNA
aaacaacacagcttctacgacagtaccttcc

SEQ ID NO: 239 - DNA, 57 bp, synthetic construct
  source 1..57: mol_type = other DNA
atgggttccagaagataataataaaggacaacaattttctcccctgagggacgtcta

SEQ ID NO: 240 - DNA, 51 bp, synthetic construct
  source 1..51: mol_type = other DNA
catcagattgaagagtaataataagatggagctacgacggatcccgaagtt

SEQ ID NO: 241 - DNA, 39 bp, synthetic construct; identical to SEQ ID NO: 137
  source 1..39: mol_type = other DNA

SEQ ID NO: 242 - DNA, 39 bp, synthetic construct; identical to SEQ ID NO: 138
  source 1..39: mol_type = other DNA

SEQ ID NO: 243 - DNA, 42 bp, synthetic construct
  source 1..42: mol_type = other DNA
atggcgaagattatgtaataataaagcaagcagcctgtttac

SEQ ID NO: 244 - DNA, 39 bp, synthetic construct; identical to SEQ ID NO: 140
  source 1..39: mol_type = other DNA

SEQ ID NO: 245 - DNA, 39 bp, synthetic construct; identical to SEQ ID NO: 141
  source 1..39: mol_type = other DNA

SEQ ID NO: 246 - DNA, 69 bp, synthetic construct; identical to SEQ ID NO: 142
  source 1..69: mol_type = other DNA

SEQ ID NO: 247 - DNA, 69 bp, synthetic construct; identical to SEQ ID NO: 143
  source 1..69: mol_type = other DNA

SEQ ID NO: 248 - DNA, 69 bp, synthetic construct; identical to SEQ ID NO: 144
  source 1..69: mol_type = other DNA

SEQ ID NO: 249 - DNA, 69 bp, synthetic construct; identical to SEQ ID NO: 145
  source 1..69: mol_type = other DNA

SEQ ID NO: 250 - DNA, 69 bp, synthetic construct; identical to SEQ ID NO: 146
  source 1..69: mol_type = other DNA

SEQ ID NO: 251 - DNA, 69 bp, synthetic construct; identical to SEQ ID NO: 147
  source 1..69: mol_type = other DNA

SEQ ID NO: 252 - DNA, 78 bp, synthetic construct
  source 1..78: mol_type = other DNA
tctaacttgccatctttggcttaatatataactgtctagaaataaagagtatcatctttcaaaacagtgttctacttt

SEQ ID NO: 253 - DNA, 69 bp, synthetic construct; identical to SEQ ID NO: 149
  source 1..69: mol_type = other DNA

SEQ ID NO: 254 - DNA, 39 bp, synthetic construct; identical to SEQ ID NO: 150
  source 1..39: mol_type = other DNA

SEQ ID NO: 255 - DNA, 39 bp, synthetic construct; identical to SEQ ID NO: 151
  source 1..39: mol_type = other DNA

SEQ ID NO: 256 - DNA, 39 bp, synthetic construct; identical to SEQ ID NO: 152
  source 1..39: mol_type = other DNA

SEQ ID NO: 257 - DNA, 54 bp, synthetic construct
  source 1..54: mol_type = other DNA
gttgccgatgcgcaaatacagagagaaacgtaataataaaacgagtctgacgat

SEQ ID NO: 258 - DNA, 39 bp, synthetic construct; identical to SEQ ID NO: 154
  source 1..39: mol_type = other DNA

SEQ ID NO: 259 - DNA, 39 bp, synthetic construct; identical to SEQ ID NO: 155
  source 1..39: mol_type = other DNA

SEQ ID NO: 260 - DNA, 60 bp, synthetic construct
  source 1..60: mol_type = other DNA
ctgtacgacctgctgtaataataactggacgtgcatggtcgaagtaatgaagagccgaga

SEQ ID NO: 261 - DNA, 39 bp, synthetic construct; identical to SEQ ID NO: 157
  source 1..39: mol_type = other DNA

SEQ ID NO: 262 - DNA, 39 bp, synthetic construct; identical to SEQ ID NO: 158
  source 1..39: mol_type = other DNA

SEQ ID NO: 263 - DNA, 48 bp, synthetic construct
  source 1..48: mol_type = other DNA
tcggaaacaatgagataataataaaatgaggaaggtttgcgggataaa

SEQ ID NO: 264 - DNA, 48 bp, synthetic construct
  source 1..48: mol_type = other DNA
gaagccaaacaagaataataataacgtgactatgaagagaccatcgaa

SEQ ID NO: 265 - DNA, 39 bp, synthetic construct; identical to SEQ ID NO: 161
  source 1..39: mol_type = other DNA

SEQ ID NO: 266 - DNA, 39 bp, synthetic construct; identical to SEQ ID NO: 162
  source 1..39: mol_type = other DNA

SEQ ID NO: 267 - DNA, 54 bp, synthetic construct
  source 1..54: mol_type = other DNA
tttattgccacagcaaatgcaggtaaagcttaataataagatattttctcggtt

SEQ ID NO: 268 - DNA, 39 bp, synthetic construct; identical to SEQ ID NO: 164
  source 1..39: mol_type = other DNA

SEQ ID NO: 269 - DNA, 39 bp, synthetic construct; identical to SEQ ID NO: 165
  source 1..39: mol_type = other DNA

SEQ ID NO: 270 - DNA, 39 bp, synthetic construct; identical to SEQ ID NO: 166
  source 1..39: mol_type = other DNA

SEQ ID NO: 271 - DNA, 39 bp, synthetic construct; identical to SEQ ID NO: 167
  source 1..39: mol_type = other DNA

SEQ ID NO: 272 - DNA, 39 bp, synthetic construct; identical to SEQ ID NO: 168
  source 1..39: mol_type = other DNA

SEQ ID NO: 273 - DNA, 39 bp, synthetic construct; identical to SEQ ID NO: 169
  source 1..39: mol_type = other DNA

SEQ ID NO: 274 - DNA, 39 bp, synthetic construct; identical to SEQ ID NO: 170
  source 1..39: mol_type = other DNA

SEQ ID NO: 275 - DNA, 39 bp, synthetic construct; identical to SEQ ID NO: 171
  source 1..39: mol_type = other DNA

SEQ ID NO: 276 - DNA, 39 bp, synthetic construct; identical to SEQ ID NO: 172
  source 1..39: mol_type = other DNA

SEQ ID NO: 277 - DNA, 39 bp, synthetic construct; identical to SEQ ID NO: 173
  source 1..39: mol_type = other DNA

SEQ ID NO: 278 - DNA, 39 bp, synthetic construct; identical to SEQ ID NO: 174
  source 1..39: mol_type = other DNA

SEQ ID NO: 279 - DNA, 45 bp, synthetic construct
  source 1..45: mol_type = other DNA
ttgaagtcagacactgatgtataataataaaatgcgtcaacagta

SEQ ID NO: 280 - DNA, 63 bp, synthetic construct
  source 1..63: mol_type = other DNA
ctttccagaacaatccgtacacaaggttccttcctaagataataataactgaccattacaaga

SEQ ID NO: 281 - DNA, 45 bp, synthetic construct
  source 1..45: mol_type = other DNA
ttggacgataatgactaataataaccattgccggacacagacagc

SEQ ID NO: 282 - DNA, 12 bp, synthetic construct
  source 1..12: mol_type = other DNA
acacccgtacac

SEQ ID NO: 283 - DNA, 16 bp, synthetic construct
  source 1..16: mol_type = other DNA
cggagtaaccgcgccg

SEQ ID NO: 284 - DNA, 11 bp, synthetic construct
  source 1..11: mol_type = other DNA
cggggttttct

SEQ ID NO: 285 - DNA, 10 bp, synthetic construct
  source 1..10: mol_type = other DNA
cgcggttttt

SEQ ID NO: 286 - DNA, 12 bp, synthetic construct
  source 1..12: mol_type = other DNA
acacccgtacac

SEQ ID NO: 287 - DNA, 11 bp, synthetic construct
  source 1..11: mol_type = other DNA
attttgcgggg

SEQ ID NO: 288 - DNA, 17 bp, synthetic construct
  source 1..17: mol_type = other DNA
cgccacctatcattttt

SEQ ID NO: 289 - DNA, 44 bp, synthetic construct
  source 1..44: mol_type = other DNA
gtaagaaaatcaatggaggcagcagaaataggtatggctgcgca

SEQ ID NO: 290 - DNA, 42 bp, synthetic construct
  source 1..42: mol_type = other DNA
gcttcagtagggcggtaacttcttcagaagagaacggacccg

SEQ ID NO: 291 - DNA, 20 bp, synthetic construct
  source 1..20: mol_type = other DNA
acaaacgtctaaatccaaaa

SEQ ID NO: 292 - DNA, 55 bp, synthetic construct
  source 1..55: mol_type = other DNA
taataattgtaaagtgcgttctccagataaatgtttttcatgtgcactcgataaa

SEQ ID NO: 293 - DNA, 33 bp, synthetic construct
  source 1..33: mol_type = other DNA
tttcctcatcttcagatgacgatgttagcagcg

SEQ ID NO: 294 - DNA, 28 bp, synthetic construct
  source 1..28: mol_type = other DNA
gctcattatttggaaacaacacagcttc

SEQ ID NO: 295 - DNA, 39 bp, synthetic construct
  source 1..39: mol_type = other DNA
cttctttgaagaagtaaataaatataaatagagagaaat

SEQ ID NO: 296 - DNA, 39 bp, synthetic construct
  source 1..39: mol_type = other DNA
tatataactgtctagaaataaagagtatcatctttcaaa

SEQ ID NO: 297 - DNA, 39 bp, synthetic construct
  source 1..39: mol_type = other DNA
ctccatgcatgctacatagtaactacgtaaatcacctgc

SEQ ID NO: 298 - DNA, 39 bp, synthetic construct; identical to SEQ ID NO: 296
  source 1..39: mol_type = other DNA

SEQ ID NO: 299 - DNA, 39 bp, synthetic construct
  source 1..39: mol_type = other DNA
tgaactttttgtataacatcattggtatacaagctttat

SEQ ID NO: 300 - DNA, 39 bp, synthetic construct; identical to SEQ ID NO: 296
  source 1..39: mol_type = other DNA

SEQ ID NO: 301 - DNA, 39 bp, synthetic construct
  source 1..39: mol_type = other DNA
ttatcataaataccaacttttgcgtcataaaagtacaaa

SEQ ID NO: 302 - DNA, 39 bp, synthetic construct; identical to SEQ ID NO: 296
  source 1..39: mol_type = other DNA

SEQ ID NO: 303 - DNA, 39 bp, synthetic construct
  source 1..39: mol_type = other DNA
acgttaggacttctttaattccctcttttatgctttagt

SEQ ID NO: 304 - DNA, 39 bp, synthetic construct; identical to SEQ ID NO: 296
  source 1..39: mol_type = other DNA

SEQ ID NO: 305 - DNA, 39 bp, synthetic construct
  source 1..39: mol_type = other DNA
agcgcagccagatgcgacaaaaacttaaaggcgcggctc

SEQ ID NO: 306 - DNA, 39 bp, synthetic construct; identical to SEQ ID NO: 296
  source 1..39: mol_type = other DNA

SEQ ID NO: 307 - DNA, 39 bp, synthetic construct
  source 1..39: mol_type = other DNA
gccttatatatgaataattccaactgaaagaatccaata

SEQ ID NO: 308 - DNA, 39 bp, synthetic construct; identical to SEQ ID NO: 296
  source 1..39: mol_type = other DNA

SEQ ID NO: 309 - DNA, 39 bp, synthetic construct
  source 1..39: mol_type = other DNA
actccttttacttcggttgtgaaagaaagttgacattat

SEQ ID NO: 310 - DNA, 39 bp, synthetic construct; identical to SEQ ID NO: 296
  source 1..39: mol_type = other DNA

SEQ ID NO: 311 - DNA, 100 bp, Saccharomyces cerevisiae
  source 1..100: mol_type = other DNA
cttctttgaagaagtaaataaatataaatagagagaaatatgacgtatcaatattatttatggcgctggtataattcaatagcatctaatagcgacttgg

SEQ ID NO: 312 - DNA, 100 bp, Saccharomyces cerevisiae
  source 1..100: mol_type = other DNA
ctccatgcatgctacatagtaactacgtaaatcacctgctacctctctgttctttcgtcccgcctcgaggttgtgctgcttttgtacctattgtggctgt

SEQ ID NO: 313 - DNA, 100 bp, Saccharomyces cerevisiae
  source 1..100: mol_type = other DNA
tgaactttttgtataacatcattggtatacaagctttataaataaatgttaaatagttaatgaaataaaaaatcacatttgacgaattcggaagacaagt

SEQ ID NO: 314 - DNA, 100 bp, Saccharomyces cerevisiae
  source 1..100: mol_type = other DNA
ttatcataaataccaacttttgcgtcataaaagtacaaagtaactaccaatacataatcttcaacgtactgtccataggttgtcgtggccaaagatgata

SEQ ID NO: 315 - DNA, 100 bp, Saccharomyces cerevisiae
  source 1..100: mol_type = other DNA
acgttaggacttctttaattccctcttttatgctttagtatcgtcatattctttcatacccttccttataagctttttatataaaatatcccaccgggaa

SEQ ID NO: 316 - DNA, 100 bp, Saccharomyces cerevisiae
  source 1..100: mol_type = other DNA
agcgcagccagatgcgacaaaaacttaaaggcgcggctcagcctagattacgttctttttcaggtgatggtgccatttccgcccttctcgttagagccgg

SEQ ID NO: 317 - DNA, 100 bp, Saccharomyces cerevisiae
  source 1..100: mol_type = other DNA
gccttatatatgaataattccaactgaaagaatccaataacagtgttctactttctgtttctattactattattattttaattggttatttactatataa

SEQ ID NO: 318 - DNA, 100 bp, Saccharomyces cerevisiae
  source 1..100: mol_type = other DNA
actccttttacttcggttgtgaaagaaagttgacattatcgatttgggtgacacggtgattgaaaaagcaacgaccagtattatacctcttttttttatt

SEQ ID NO: 319 - DNA, 2500 bp, Saccharomyces cerevisiae
  source 1..2500: mol_type = other DNA
aattatactctatttctcaacaagtaattggttgtttggccgagcggtctaaggcgcctgattcaagaaatatcttgaccgcagttaactgtgggaatactcaggtatcgtaagatgcaagagttcgaatctcttagcaaccattatttttttcctcaacataacgagaacacacaggggcgctatcgcacagaatcaaattcgatgactggaaattttttgttaatttcagaggtcgcctgacgcatatacctttttcaactgaaaaattgggagaaaaaggaaaggtgagagcgccggaaccggcttttcatatagaatagagaagcgttcatgactaaatgcttgcatcacaatacttgaagttgacaatattatttaaggacctattgttttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatgttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggtcaaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaaatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaacacgctccagtctttgaaaacgattgaatacattagcaacgcgtccagcatttttgagatgtgtctcataaactttactcaagagttaagtactgaaaaattcgacttttatgatagttcaagtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccaaagttgattgttaaaaacgtatttatagcaaacgcaattgtaataaattcttattttgtatcttttcttcccttgtctcaatcttttatttttattttatttttcttttcttagtttctttcataacaccaagcaactaatactataacatacaataata

SEQ ID NO: 320 - DNA, 2265 bp, Saccharomyces cerevisiae
  source 1..2265: mol_type = other DNA
atggcacatgaaaagtttagtatacctgagaacttcacattagcgcagtctttgcaattgctttactctgttgtcaaaaatcaatataaaaacctagcagacctaataatcaatagcaaaggtaataaggacacagtaacgtatgggaaaatccacaaaaacttagacactttactggtatacgtcaatgaaggtctacgaaaaattgaaaagacttataccttaaagaaagggttaggaaatcttgtagttgatcatcctgaattgaggagcatcattgaggatttccaaattcttggccaggatataaggatcgcacgtcgtaaagcagaaacgcttatggccgaaggaaacggtagtccctctttgtcatcctcctcttccgttttgggccttgggacaggaaatggtttgcgttttccaaaactttggagaatgggttccaaaagagataaattgaaagaagctgacgaaaaggaggctaaaatcaacaaacaggctgataatatcagacgtgctaggaaattagaagaagagaaaaagttaggagcaaaacttcagtatgagagggacttagaacttcaaagggaaaaattgatcgagttaaaggttaaagaaaaggtcgaatttgaagtggcccaaaagttggaggaggagagagttaagagagaggaagaagaacgcaaacatagagaacaagcggagaaaaaacgtatatcaaccctgaaacatgagaggaaaacaaattacaaatcaagggctagcctcgacaacttctcttccagtagcaaatcttctggaaagattgacaactccctaattaaaagaagatcacttgacgttgtacggacttcaggcgaaagagtacgaacgccagtaagaaaatcaatggaggcagcagaaataggtatggctgcgcagttagcctggtctcagtatcaaaacggggcaaatcattcaaaagtgagtaacaacggatcgcattcaaatgaacttcagatacgttataagccaacaccacctttgaaaaaaaggtatgactacaaaaagccaacagttaatcgaccgattataaaatcacctactcttaacaggcaaaacagcaagtcttcacggaatatacccacaaatagcaagctgaaggctagtaaatccaatactaacaaagtttctcgtaggaatgagcaaaatttggagcctagttcaccagtactcgtttcagcgacagcggtacctgctgaatccaaacccatgcgttctaaaagtggtacacccgataaggaaagctctgctagttcatccctcgattccagaaaggaggatatattaaaatcagtgcagggagtagatcgaaacgcttgtgaacagatacttaatgaaattcttgttacggatgaaaaagtttattgggaggatatcgcaggcttacggaatgcgaagaattctttgaaagaagcagtggtttacccatttctgcggcctgacttattcaaagggctaagagaacctgttagaggtatgcttttgttcggcccaccaggtacaggtaagacaatgattgctaaggcggttgcgacagagtccaattccacattttttagtgtaagtgcatcttctttgctatcgaaatacctcggtgaatctgagaagctggttagagcattgttttacatggccaaaaaattgtcaccctcaattattttcatagatgagattgactctatgttaacagctcgctctgataatgaaaatgagtcctcgaggagaattaaaacagaacttcttattcagtggtcctccctatctagtgctactgcacaatcggaagaccgaaataatacgcttgacagcagagtacttgtcttaggagcaacgaatttgccatgggcgattgatgatgcagcaagaagacgattttcacggaaactatacattcctttaccagattatgaaactagactatatcatttgaagagattaatggccaagcagaaaaatagcctgcaagatttagactatgaattaataacagaaatgaccgaggggttttctggttctgatcttacgtcattagccaaagaagcggcaatggaacctattagagatttaggagataagttgatgtttgcggattttgataaaatcagaggaattgaaataaaagattttcagaatgctctgttgacaataaaaaagagcgtgtcgtcagaatctctacaaaaatatgaggagtggtctagtaaattcggtagtaatggttcctga

SEQ ID NO: 321 - DNA, 2500 bp, Saccharomyces cerevisiae
  source 1..2500: mol_type = other DNA
ttcttcaaaggcctttcttaagttagactctgattcaccagccatttttgacatgacttctggaccattaattaagaaaaaaaaggcaccggtctcattagcaacagctcttgccatcaaggttttaccagtaccagggggaccatacatcaaaacacctcttggtggcttgataccgatagccttgaacaactgaggatgtctcaatggtagttcaaccatttccctaatttgagccatttgcttacgacaaccaccaatatcatcgtaacccacctcattcatattattttcttcatcttccctgttgattggctcaccttcccagtgaataatagtatcctgagcaacgacggcatattcttcaggttcgacatccacaaccttgaattcgacttgtctcataccgcctctgacaacaaaatggtcgcctttcctcactggtctgtaggcttccacaaaataaggcttcaaaaaaacatcgaaaagattaccagttataccttcgatcgtatcagcaattggtaacacagaaattctagtagcgtatttgatatcggggcaaggatgaattgtaactaaatcacccagcctaatacgtaaattgttacgaactacacggtttatcctgcatgctccgtcttctaattcgtcatcgatcaacacaattaagacagtatcttttctcttcttgcccttaacgagaacggtgtcaccacggaacaattccaatttgtccatggtgttagaattaatagcaatgacagaattgtcatcattgatagcatcgtccaccaaaagcatgttgtccttcttctttcttcttaaaatggcggtggcggttttatcctcctcacgagggtcgacacccgaggcgtccaaaagtggtttatgttcttcacccatgatttgtatatctgtcttgtagttgagccagttatcgagtcaatggcgttgagtcaaatcttaatagaactagattaattgactacatcaactgcggatctcttaaatacacggccctgatatatgtccttttttgccaccgaattcacattcgaggggttgccgggtagtgcgcaaaaggcggtaggttaaaaaggaaggagttagagctgttcataaggtatcgatagcaacgatggctagtaacatgattgttagcattatagagtggatatgaatggaagcaaacacacccatatgtataaacttaaatatgaaaagaaaacgctaaaagaaatcatagcatgcattactctaatcggagccttctagtttggcaagcaattccttgtgtagcttgcccaacttatcgaagtccgtttcttgggctggccaccctacaatcaaaccacttttctcatctctcttaggaatcaaatggaagtggacgtggtcgacttcttgatgcgcaattttaccattattctgcaacacattataagtgtccaacttcattgccttggccagtctcttggcaatcggcatagcatcggtaaggaattcgtccgggatgtcatgcaacttcgcaccatggtacttaggaatgattaaagcatgaccttcagcagtaggttggatgtccaagaaagcatacgagtactttgtttcaatcaatttgaaggatggaatttcgcctgttgcatagcggatcgattgttagtattctgctttatgtatatgtactacatagagactatcgccatcattggtgtgagtgtaagctctttattagtaatgtgacatacttttaataatcttgcaaaaaatacaggcagcatcaagcgtagcaggagcagacatttttgttgttgttaggtacggtgccgatatcaatggctccatcaatgtttcttaaaagtctggaaggcgtgctgattgtccttttacttcataatgttctccgctttccagtcgaacacacatcacattacacgatcgtacagcaatgcttcagtagggcggtaacttcttcagaagagaacggacccgccattatttctgcaaggaatattctgtaacgattcgcaccatatcttaaagcttctcaagcttactgttttatgtgacctccttttgtacttttcaagttctgtagacaagtgtcaattcaacctttgaggctacgtggccaacaaggtcttgcgcgatttaccggtgtttcctttgcgccccctgctcatgccatcaaaacggcacgctgttttgtagaagcctccatgcgtctcctatttgggtccttgcaaaatagtctaacgaaagcgcggagaattaaccggaaaaaggaagttgccacactccccttactaatccacacctaggatctcctttactcgtattttgaatatctacgaaaacactcacatacgctcatcccatatatatatatatatatatacggatatattgatagtcggatactacgtggatatggatacacatgtaaacggttgtgttaccctaaagaaacggaggtcagttaacaacacttttacag

SEQ ID NO: 322 - DNA, 3189 bp, Saccharomyces cerevisiae
  source 1..3189: mol_type = other DNA
atgtctgagaacaccacagccccttctgataacataaccaatgaacagagacttccgtctggacccaaagatgacgtagatacgttagcacttaccagtgctcaaaatcaagctaatagtttgaggaagctagatacagatgcgaacgctaaagctttgccatccatcacagacataccagtcagtgatgattctgatataaaacgacaagttggctctggttttggttcaaatccgcttcatataaaagattccgaggcctttccacattcatctattgaagccctaaaagagggtatggacaaagtcacaaaacaatgcaacgatttgaagacggctttgctttccaaagacacttccctcactgattctgtgcaggatttgtttaactcattaaaagttttatcacacaaccaatcagttttggaaaataagctggatgatgtcatgaagaatcaagtaaatactgacatattggtcaataatttgaatgaacgattgaacaaattatcgacaatgttgcaaaatacttcgaaagtaaatcactctaaccttctcatagaaaattcatccaacaataccagttcacagcataatacctcttcttctcgaaggggacctggcaggccaagaaaagatgcctcgacctccacaatgaataagttagtgtcaaacgcagcctcggtcaatctcaaaagtgcatccaatcagggtgctcctttctcaccagtaaatattactttaccgactgccgtagtacaaacgtctaaatccaaaaggtattttgtggaaccatcaacgaaacaagagtcgcttttattatctgccccttcatcatcaagagatgatgctgatatgtccttgacttccgtaccgcaaagaacgaacaatgaaaatggtaaagagcgaccatccactgctaattctagctccatcacaccaacacctgttacgccgaacaacttaattcaaatcaaaagaaaaagaggcaggcccccaaaaaagagaacagtggaaacaatgatatccaattccacggatacgatagataagtcagatgcatctaatcggattaaaaacgagattccaataaattccttgcttccatcttccaaatttcatcaaataccatcatctccatccaatcctgtgtcacaaccggctccggttcggacttcaaggtcagccacacaagaaatagacattaaaagtttagaactggcctctctaatttcaactaatggtgatccaaacgcagaggattcgaatactactgatactgttcataataacgtggaaggaaaggtaaatgttgaagaaaataaaaccgagaaggagaaaataataaccatcaaatcatccagcgaaaatagtggtaataatacgaccaataataataatactgacaacgtcattaaattttcagctaattcagatatcaatagtgatattcgccgattaatggttaacgatcagttttcattaagttatgacgccagcggtaatattacggtcaaattaccacccgtttcctctccagcagcagcaacagctgccgctgctgcggcggttacgtcagagatgaatagacaacaaagagaattagataaaagacgtgattcaagggagaaaatgcttgttaatatgaaatataacgatcgcgataaagcaaagtcatttatggagtctaataagaaactcttaaaagcaatgaaagaagaagagaggaggaaaagaatgacttcgataatacatgataatcacttgaacttaaatttgaatgagatctccacccgttcaaagataaaaagtgcagaaaaaccaacaactaaaggttcttcaatgtcgccaaaaccaagatcggcctccatcagcggcatttcagaccaccaacaggaaggatatcaaccattagagcaagaaaaactcgtcgatattgacaatgagggctcaaacgcaaacagcgactctctcaagatgggtctaaccatatccgctgccgatacggttcacaaagttggaatacagtccatgctaaattctggggaagaggcaattaccaaggagaatgcagaatatgaaagaaagaccccgggagatgaagaaactaccacgttcgttccattagaaaactcacaaccttcggacacgataaggaaaagaacagcaggcgatgacggtgcattggatcaaacagagaatacgagcatatcaccaaagaagagacgcacagaagatcatacaaaaggtgaagaagatgagggagaaaggggtgttggtaatagcggaacgctggccaccgtggagaatgtctcaggagacatctccgcagatttatccaaaggtacttcatctatccataacgatactgagtctgctaacgatagtagcaatggcaacggaaacctcggtttgggtacagagtcacgcaatacattgttaacggcaactcccattgaattaatctgccgggaaggcttcttctaccggagagatattccggacgttcccattaccacaggagcgtacctggaatttaaattcaaggcgaaagaagaggaattgatcaactctagcatcaatgaggaggattatgctgctaaatcaaagcatgaaaaaatgaatgcgcatttcttcaagcctgatattcaagaagagacggaacttgcctttgaaattttgagcaagacaacgctgacagagaaatacgttaacagtttggagtacttcttgatggaattcaggtgggagaataaactagttggtctaggcttaaaacttcgggaatccaaaagaacctggcaaaggagaaaggcgttatttgcccttttcgaattttggagagaccaatcaagagacaaaagaagattccacaactacaccatcctgcatgcggtgaaagagatggaaaactacagaatatttattaatcgatccgtttcatggttctacaaccatattaccctgctaaaaatgatcctctacgacctctgcgacaatgtgaccactcaatggagagagtggatgtttccccataacgaaacactgccggcattgggtcaggacggcattaacgaagacaatctgaatgaaaccatcgacaacatgttaatttttgacttccttgatgacggttcagaaaacaaccaggtcaaatattccagaatcataccgccagatatccgttaa

SEQ ID NO: 323 - DNA, 1416 bp, Saccharomyces cerevisiae
  source 1..1416: mol_type = other DNA
atgagcatttgtccacatatacagcaagtatttcagaatgaaaagtctaaagatggggttctaaaaacatgcaatgctgccaggtatatattaaatcattccgtacccaaggaaaaattcttaaacaccatgaaatgtggtacatgccacgaaataaactctggtgcaactttcatgtgtctacaatgtggattttgtggatgttggaaccattcgcattttctctctcacagtaaacagattggtcacatatttggtatcaactcaaataatggccttttattttgcttcaaatgtgaggactatatagggaatatcgatctgattaacgatgctatcctagcgaagtattgggacgacgtgtgcacaaagaccatggttcctagcatggaaagaagagatgggctttctggcctgatcaacatgggatccacttgtttcatgagtagtattctccaatgtctaatccataacccttactttattaggcactcaatgagtcaaattcattctaataattgtaaagtgcgttctccagataaatgtttttcatgtgcactcgataaaattgttcatgaactttatggagcgctgaatacaaagcaagcttcttcgtcatctacatctactaatcggcaaaccggattcatatatcttttaacttgtgcctggaaaatcaatcaaaatctagcagggtattcacaacaagatgctcatgaattttggcagtttataattaaccaaatccaccaaagctatgttcttgatttgccaaatgccaaggaagtcagcagagcaaataataagcagtgtgaatgcatagtgcatactgtgtttgagggctccttggaaagttctattgtgtgtccaggctgtcaaaataattcaaagacaaccattgatccattcttggatctttctctggatatcaaggataagaaaaaactttatgaatgtcttgacagtttccataaaaaagaacagttgaaggatttcaactatcattgtggggagtgtaacagcactcaagatgcaataaagcaactaggcatacacaaattaccatcggttttggttttgcaattgaaaagattcgaacacctacttaatggaagtaacagaaaactagacgattttattgaatttccaacttatttaaatatgaaaaattactgttcaacgaaggaaaaagataagcattctgaaaatggcaaggttccagacattatttacgaattaatcggtattgtttcccacaaggggacggttaatgagggacattatattgcattttgtaaaatttctggagggcaatggtttaaattcaatgattccatggtctcctctatatctcaagaagaggttttaaaggaacaggcatatttattattctacaccattcgtcaagtaaattga

SEQ ID NO: 324 - DNA, 2061 bp, Saccharomyces cerevisiae
  source 1..2061: mol_type = other DNA
atgaccgatatcacacccgtacagaacgatgtggatgtcaatggtaataatgtcaatgacgacgtttccagtaatctaaagaggcctatagatcaaggggatccttcgaatggactcgcagaagaagaaaaccccgccaataaccagttgcatctcaaaaaggctagactggatggagatgctctaacatcatcgcctgctggacttgcagagaacggtattgaaggcgccaccctggcggctaacggggaaaatgggtataacgccaccggaagtggcgcagaagacgaacagcaggggttgaagaaggaagaaggaggacaaggtaccaaacaagaggatttagatgaaaactcaaaacaagaacttccgatggaggttccaaaggaacctgcccctgctcctcctccagagcccgatatgaataatctccctcagaatccaataccaaagcaccagcagaaacatgcattgcttgcgattaaagctgtcaaacgcttgaaggatgcgagaccctttctacaacctgttgacccagtgaaattggatattcccttttactttaactacataaagaggccaatggacttgtctactatagagaggaagttgaacgtaggcgcttatgaagttccggagcaaatcacggaggatttcaatctcatggttaacaacagtattaaattcaacggtccaaatgcgggcatatcacaaatggcaagaaacatacaagcttctttcgagaaacatatgctaaatatgcctgctaaggatgctccacctgtaatagccaagggacggcggtctagtgcccaagaggatgccccaattgtaattagacgagcccaaactcataatgggaggccgaaaaggactatacatccgccgaaatcaaaggatatttatccttatgaatcgaagaaaccgaaatccaaaagactacaacaagcaatgaaattttgtcagagtgtgctaaaggaattgatggccaagaagcacgcctcttataactacccatttttggaaccagtagacccagtttctatgaatttgccgacttatttcgattatgttaaagagccaatggatttaggcacaatcgccaagaaattaaatgactggcagtatcaaacaatggaggattttgagagagacgtgaggttggtctttaaaaactgctacacgttcaatccggatggcacgatcgttaatatgatgggtcatcgtctagaggaagttttcaattccaaatgggcggataggcctaatttggatgactacgattccgatgaagattcgaggacccaaggcgactacgacgattatgaatctgagtattcagagtctgacatcgatgaaactataattacaaatccagccatccagtatttggaagaacaacttgctcggatgaaagtggagttgcaacaattaaaaaagcaagaactggaaaaaataagaaaagagaggcgcttagcacgtggatcaaagaaacgcggcaaaagatcgaagggaaggagtgggtctaagaacgcttcttcgaaaggaaggcgagataaaaagaataaattgaaaacagtagtgacatatgatatgaaacgtatcattacagagaggatcaatgatttaccaacttccaaattagaaagagcaatcgacataataaaaaaatccatgcccaatatttctgaagacgatgaagtagaacttgacctcgacactttagataatcacaccatcttaacattgtacaacactttctttagacaatatgaaagctcatccggtgcttctaacggtttggacggtacttcaggtgttacgcgagatgcttcgtccttgtcgcctacaagtgcgggaagcagaaagagaagatctaaggcattaagccaagaggagcagagtaggcagatagaaaagataaaaaataaactagctatcttagacagtgcttcacctctgagccaaaacggctccccaggccaaattcaaagcgctgcacacaacgggttttcctcatcttcagatgacgatgttagcagcgaaagtgaagaagagtga

SEQ ID NO: 325 - DNA, 2880 bp, Saccharomyces cerevisiae
  source 1..2880: mol_type = other DNA
atgtttggcaacaatagaccaatgtttggagggagcaacctttcctttggatcaaatacgtcatccttcggtgggcaacaatcgcaacaaccaaactctctttttggaaatagcaataacaacaacaattccaccagtaacaatgcccaatcaggatttggtggattcacttctgccgctggcagtaatagtaacagtttatttggaaataacaatactcaaaataatggggcatttggccagtcaatgggtgccacccaaaactcaccatttgggtcgttaaactcctcaaatgccagtaatgggaacacttttggaggatccagttcaatgggttcttttggtggtaataccaataatgcatttaataataacagtaacagtaccaattccccgtttggtttcaataaaccgaatacaggagggaccttgtttggttcgcagaataataatagtgcaggtacttctagtttatttggcggccaaagtacaagtaccactggcacattcggaaacaccggaagtagtttcggaacaggcttaaatggcaacggctctaacatatttggggcagggaacaactcacaaagcaatactaccggcagcttgtttggcaaccaacaatcttcagcgtttgggacaaataatcaacaaggaagtctatttggacagcaatctcaaaacaccaataatgcgtttggtaatcaaaatcagctggggggcagttccttcggatcaaaaccagttggttcagggtcgctgtttggccagagcaacaacactttaggtaatacaaccaataatagaaatggattgtttggtcaaatgaattccagtaaccaaggcagttctaacagtggattgtttggacaaaactcgatgaacagcagcactcaaggggtatttggacaaaacaataatcaaatgcaaattaatggcaataacaacaacagtttatttggaaaggcaaacaccttttcaaattccgcatcaggaggtttatttggccaaaataatcaacagcaagggtccgggctgtttggtcaaaattctcagacaagcggtagtagtggactttttgggcagaataatcagaaacagcccaatacttttacccaatctaatacaggaataggtctctttggacagaacaacaaccagcaacaacaatctacaggtttgtttggagccaaaccagcaggtaccacaggatctcttttcggtggtaattcatcaacccagcctaactctcttttcggtactaccaatgtacctacctctaatacacagtcacaacaaggaaatagtctttttggtgccacgaagctgaccaacatgccctttggaggaaatcccactgcaaaccagtcagggagtggaaacagtctattcggaactaaacctgcctccactaccggctcattatttggaaacaacacagcttctacgacagtaccttccacgaatggattgtttggtaacaacgctaacaattcaactagcactacgaatacgggacttttcggtgcaaagcctgattctcagtccaaacctgcattagggggagggttattcggcaattcaaactcgaattcttccacaatcggccaaaacaaaccagtttttggaggcacaacccaaaatacaggactctttggcgccaccggcacgaactcttcagcagttggttcaactggtaaactttttggccagaataataatacgcttaatgttggtacacaaaatgtaccacctgtgaacaataccacccaaaacgcccttttgggtacaacggcagttccttccctacaacaagccccagtaactaatgaacagcttttttccaaaatatcaatccctaactctattacaaatccagttaaagcaacaacttcaaaagtgaacgcagatatgaaaagaaatagtagcctcacgtctgcctatagacttgccccaaagccgttatttgctccctcttcgaatggcgatgctaaatttcaaaagtggggcaagacactggaaagaagtgatagaggaagcagtaccagcaattctattacggacccagaatcaagctatctaaattcaaacgacttgttgtttgatccagatagaagatatttgaaacatctggtgattaaaaataataagaacttaaatgtcattaaccataatgatgatgaagcaagcaaagttaaattagtgacgtttacaacagaatcagcttcaaaagatgaccaagcctcatcaagcattgctgcttcaaaattaactgaaaaagcacattctcctcagactgacctaaaagatgatcatgatgaaagcactcctgatcctcaatcgaaatctccaaatggttccacctctataccaatgattgagaatgaaaagattagcagcaaagttcccggcctattgagcaacgacgttacctttttcaagaataactactacatttcaccttccatagaaacgcttggcaataagtcattaattgaacttcgtaaaataaacaacctagtcattggtcacagaaattatggtaaagtcgagtttctggagcccgttgatttgttgaatactcctttggatactttatgcggggatcttgtcacctttggaccaaaatcatgttcaatatatgaaaactgttccataaagccagaaaagggcgaaggcattaatgtacgttgtagagtgactttatattcctgttttcctattgacaaagaaacaaggaaacctataaagaatataacacatcctctactgaaaagaagtatagccaaactaaaagaaaacccagtgtacaagtttgaaagctatgaccccgtaacaggcacctatagttacaccatagatcacccagttttaacttaa

SEQ ID NO: 326 - DNA, 1368 bp, Rasamsonia emersonii
  source 1..1368: mol_type = other DNA
atgctaagaagagctttactattatcttctagtgctattttagctgttaaagcacagcaagccggtactgcaacagcagaaaatcatccaccattaacatggcaagagtgtacagcacccgggagttgtaccacccaaaatggtgcagttgtcttggacgcaaattggcgttgggtccacgacgttaatggttatacaaattgctacactggaaatacctgggacccaacctattgtcctgatgacgaaacctgtgctcagaactgcgctctggatggtgcagactacgaagggacgtacggggttacgtctagcggatcatccctgaagcttaactttgttactggttctaatgtaggctcccgtttatacctcttgcaagatgattctacttatcaaatcttcaagttgttaaaccgcgagttcagtttcgatgttgacgtttctaatcttccttgtggtttgaacggcgcactttacttcgtggctatggatgcagatggaggtgtttcaaagtacccaaacaataaggcgggtgccaaatacggaacgggttattgcgattcccaatgcccccgtgatctgaagtttatagatggcgaggccaatgtagaaggctggcagccatcatccaacaacgctaacacagggataggtgatcatggctcctgttgcgctgaaatggatgtgtgggaggctaactccatctcaaatgctgtaactccacacccatgtgatacacccggtcaaacaatgtgttcaggcgacgattgtggtggcacttactcaaatgaccgctacgcaggcacttgcgatccagacggttgtgattttaatccataccgtatgggaaacacgagtttctatggaccaggtaagataattgatacaacgaaaccgttcacggttgttactcaatttttaacagatgatggcaccgataccggcacactctcggaaattaaaagattctacattcagaatagtaatgttattccgcaacccaattctgatataagtggtgttacaggcaatagcattactacagagttttgtactgctcagaagcaagccttcggggataccgatgacttctctcaacatggaggcctggcaaagatgggggcggcaatgcaacaaggaatggtcctcgtgatgtcactgtgggatgactatgctgctcagatgctgtggctcgattcagactatcccactgacgccgatccaaccacccctggaatagccagaggtacttgtcctacagattcgggagtgccaagtgatgttgaatcacaatcaccgaactcttacgtgacgtacagtaacatcaagtttggcccaatcaactcaacttttactgcttcttaa

(19) United States
(12) Patent Application Publication
(10) Pub. No.: US 2024/0425834 A1
(43) Pub. Date: Dec. 26, 2024

(54) GENOME-WIDE RATIONALLY-DESIGNED MUTATIONS LEADING TO ENHANCED CELLOBIOHYDROLASE I PRODUCTION IN S. CEREVISIAE

(71) Applicant: Inscripta, Inc., Pleasanton, CA (US)

(72) Inventors: Eric ABBATE, Pleasanton, CA (US); Katherine KROUSE, Pleasanton, CA (US); Aaron BROOKS, Pleasanton, CA (US); Tyson SHEPHERD, Pleasanton, CA (US); Nandini KRISHNAMURTHY, Pleasanton, CA (US)

(73) Assignee: Inscripta, Inc., Pleasanton, CA (US)

(21) Appl. No.: 18/685,686

(22) PCT Filed: Aug. 24, 2022
(86) PCT No.: PCT/US2022/075396

§ 371 Date: Feb. 22, 2024

Related U.S. Application Data
(60) Provisional application No. 63/342,152, filed on May 15, 2022.
(60) Provisional application No. 63/236,268, filed on Aug. 24, 2021.

(51) Int. Cl. C12N 9/42; C07K 14/395

(52) U.S. Cl. CPC C12N 9/2437; C07K 14/395

(57) ABSTRACT

The present disclosure relates to various different types of mutations or modifications in Saccharomyces cerevisiae coding and noncoding regions leading to enhanced cellobiohydrolase I production for, e.g., supplements and nutraceuticals.

### CROSS REFERENCE TO RELATED APPLICATIONS

This application is a national stage application of International Patent Application No. PCT/US2022/075396, filed Aug. 24, 2022, which claims the benefit of U.S. Provisional Patent Application No. 63/236,268, filed Aug. 24, 2021, and U.S. Provisional Patent Application No. 63/342,152, filed May 15, 2022, the contents of which are incorporated herein by reference in their entireties.

### INCORPORATION OF SEQUENCE LISTING

A sequence listing contained in the file named P35217WO00_110642000003 which is 341 kilobytes as measured in Microsoft Windows® and created on Aug. 24, 2022, is filed electronically herewith and incorporated by reference in its entirety.

### FIELD OF THE INVENTION

The present disclosure relates to mutations in genes in Saccharomyces cerevisiae leading to enhanced cellobiohydrolase I production.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Cellobiohydrolase I (CBH1 or CBHI) is an enzyme involved in the degradation of cellulose. The enzyme functions as an exocellulase that releases cellobiose units from the reducing-end of a cellulose chain. CBH1 along with a cocktail of other enzymes can ultimately convert cellulose to glucose. The CBH1 enzyme has significance in a consolidated bioprocessing (CBP) system, which combines multiple biological steps into a single reaction system. In this process, a microbe expresses a set of enzymes used to degrade an input feedstock (usually a waste plant material), ultimately converting it to soluble sugars. These sugars are then fermented by the microbe to produce fuels, such as ethanol, or other commercially valuable chemicals. Because of the possibility of converting waste plant material into products of value, there has been a growing effort to engineer microbes with a CBP system. The disclosed amino acid and nucleic acid sequences from S. cerevisiae that enhance CBHI production are a step in satisfying this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure provides a Saccharomyces cerevisiae cell comprising a transgene encoding a cellobiohydrolase I (CBHI) enzyme and at least one modification affecting the expression or activity of a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme and a null allele of a nucleic acid molecule encoding a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme and at least one substitution allele of a nucleic acid molecule encoding a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme and at least one synonymous edit of a nucleic acid molecule encoding a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme and at least one regulatory element modification in a nucleic acid molecule encoding a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme and at least one insertion or deletion in a nucleic acid molecule encoding a protein, where a wildtype version of the protein comprises an amino acid sequence selected from the group consisting of SEQ ID NOs: 1 to 73.

The present disclosure also provides a Saccharomyces cerevisiae cell comprising a transgene encoding a cellobiohydrolase I (CBHI) enzyme, a first modification affecting the expression or activity of a first protein, and a second modification affecting the expression or activity of a second protein, wherein a wildtype version of the first protein and a wildtype version of the second protein each comprise an amino acid sequence selected from the group consisting of SEQ ID NOs: 1 to 73. In some aspects, the cell further comprises a third modification affecting the expression or activity of a third protein, wherein a wildtype version of the third protein comprises an amino acid sequence selected from the group consisting of SEQ ID NOs: 1 to 73.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A and 1B are graphic depictions of the cellulose degradation, highlighting the enzymes in the pathway, including cellobiohydrolase I, which has been targeted for rationally-designed editing.

FIGS. 2A-2C depict three different views of an exemplary automated multi-module cell processing instrument for performing nucleic acid-guided nuclease editing.

FIG. 3 is a graph identifying the fold change over base strain for the different types of edits made to increase CBHI production.

FIG. 4 depicts the structure of the Enolase-2 promoter and the targeted sites for insertion, deletion, or substitution mutations or modifications.

FIG. 5 is an enlarged view of the structure of the Enolase-2 promoter depicting the targeted sites for insertion, deletion, or substitution mutation or modifications within the transcription factor binding sites (TFBS) in the promoter.

FIG. 6 is a graph depicting the growth of an S. cerevisiae base strain and mutated or modified strains during a time course (x-axis). Colonies from several libraries were grown in 25 mL shake flask cultures (YDP, 20 g/L) glucose, and absorbance was measured at 600 nm.

FIG. 7 is a graph depicting CBHI activity in an S. cerevisiae base strain and mutated or modified strains during a time course (x-axis). The activity of CBHI is measured through a substrate-based assay designed to measure absorbance at 405 nm (y-axis) to determine CBHI activity.

FIG. 8 is a graph showing that diverse libraries can impact many cellular functions necessary for efficient protein production, particularly production of CBHI.

It should be understood that the drawings are not necessarily to scale, and that like reference numbers refer to like features.

### DETAILED DESCRIPTION

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities. Moreover, all of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis and hybridization and ligation of polynucleotides. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Viral Vectors (Kaplift & Loewy, eds., Academic Press 1995); all of which are herein incorporated in their entirety by reference for all purposes. For mammalian/stem cell culture and methods see, e.g., Basic Cell Culture Protocols, Fourth Ed. (Helgason & Miller, eds., Humana Press 2005); Culture of Animal Cells, Seventh Ed. (Freshney, ed., Humana Press 2016); Microfluidic Cell Culture, Second Ed. (Borenstein, Vandon, Tao & Charest, eds., Elsevier Press 2018); Human Cell Culture (Hughes, ed., Humana Press 2011); 3D Cell Culture (Koledova, ed., Humana Press 2017); Cell and Tissue Culture: Laboratory Procedures in Biotechnology (Doyle & Griffiths, eds., John Wiley & Sons 1998); Essential Stem Cell Methods, (Lanza & Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza & Atala, eds., Academic Press 2013); and Handbook of Stem Cells, (Atala & Lanza, eds., Academic Press 2012). CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components-translated in an appropriate host cell.

The terms “CREATE fusion enzyme” or the terms “nickase fusion” or “nickase fusion enzyme” refer to a nucleic acid-guided nickase fused to a reverse transcriptase where the fused enzyme both binds and nicks a target sequence in a sequence-specific manner and is capable of utilizing a repair template to incorporate nucleotides into the target sequence at the site of the nick.

The terms “editing cassette”, “CREATE cassette”, “CREATE editing cassette”, “CREATE fusion editing cassette” or “CF editing cassette” refer to a nucleic acid molecule comprising a coding sequence for transcription of a guide nucleic acid or gRNA covalently linked to a coding sequence for transcription of a repair template.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

A “locus” refers to a fixed position on a chromosome. In an aspect, a locus comprises a gene. A locus can represent a single nucleotide, a few nucleotides, or a large number of nucleotides in a genomic region.

“Homology” or “identity” or “similarity” refers to sequence similarity between two peptides or, more often in the context of the present disclosure, between two nucleic acid molecules. The term “homologous region” or “homology arm” refers to a region on the repair template with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Nucleic acid-guided editing components” refers to one, some, or all of a nucleic acid-guided nuclease or nickase fusion enzyme, a guide nucleic acid and a repair template.

A “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. A promoter can be an endogenous promoter, synthetically produced, varied, or derived from a known or naturally occurring promoter sequence or other promoter sequence. Promoters may be constitutive or inducible. Examples of promoters as disclosed herein include an Enolase-2 (ENO-2) promoter, a Yeast Tat-binding Analog 6 (YTA6) promoter, an aldo-keto reductase superfamily protein (YDL124W) promoter, a Suppressor of Marl-1 protein (SUM1) promoter, a Ubiquitin Specific Peptidase 8 (USP8) promoter, a Bromodomain Factor 1 (BDF1) promoter, or a NUclear Pore (NUP100) promoter.

As used herein “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous. In an aspect, a promoter provided herein is operably linked to a heterologous nucleic acid molecule.

A “terminator” or “terminator sequence” refers to a DNA regulatory region of a gene that signals termination of transcription of the gene to an RNA polymerase. Terminators cause transcription to stop. Examples of terminators as disclosed herein include a dityrosine-deficient 1 (DIT1) terminator, a Repression Factor of Middle sporulation element (RFM1) terminator, a YHR182W terminator, a Multicopy suppressor of Ers1 Hygromycin B sensitivity (MEH1) terminator, a YBR242W terminator, a Putative serine/Threonine protein Kinase (PTK2) terminator, a YLR406C-A terminator, a Suppressor of ToM1 (STM1) terminator, or a glutathione (GSH1) terminator.

Promoters and terminators may control the rate at which a gene is transcribed and the rate at which mRNA is degraded. As a result, these elements may control net protein expression from the gene.

As used herein “allele” refers to an alternative nucleic acid sequence at a particular locus. The length of an allele can be as small as one nucleotide base. For example, a first allele can occur on one chromosome, while a second allele occurs on a second homologous chromosome, e.g., as occurs for different chromosomes of a heterozygous individual, or between different homozygous or heterozygous individuals in a population.

As used herein the terms “repair template” or “donor nucleic acid” or “donor DNA” or “homology arm” or “HA” or “homology region” or “HR” refer to 1) nucleic acid that is designed to introduce a DNA sequence modification (insertion, deletion, substitution) into a locus by homologous recombination using nucleic acid-guided nucleases, or 2) a nucleic acid that serves as a template (including a desired edit) to be incorporated into target DNA by a reverse transcriptase portion of a nickase fusion enzyme in a CREATE fusion (CF) editing system. For homology-directed repair, the repair template must have sufficient homology to the regions flanking the “cut site” or the site to be edited in the genomic target sequence. For template-directed repair, the repair template has homology to the genomic target sequence except at the position of the desired edit although synonymous edits may be present in the homologous (e.g., non-edit) regions. The length of the repair template(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein, a “mutation” refers to an inheritable genetic modification introduced into a gene to alter the expression or activity of a product encoded by the gene. Such a modification can be in any sequence region of a gene, for example, in a promoter, 5′ UTR, exon, intron, 3′ UTR, or terminator region. In an aspect, a mutation reduces, inhibits, or eliminates the expression or activity of a gene product. In an aspect, a mutation increases, elevates, strengthens, or augments the expression or activity of a gene product. In some aspects, “mutation” and “modification” may be used interchangeably in the present disclosure.

In an aspect, a mutation or modification is a “non-natural” or “non-naturally occurring” mutation or modification. As used herein, a “non-natural” or “non-naturally occurring” mutation or modification refers to a non-spontaneous mutation or modification generated via human intervention, and does not correspond to a spontaneous mutation or modification generated without human intervention. Non-limiting examples of human intervention include mutagenesis (e.g., chemical mutagenesis, ionizing radiation mutagenesis) and targeted genetic modifications (e.g., CRISPR-based methods, TALEN-based methods, zinc finger-based methods). Non-natural mutations or modifications and non-naturally occurring mutations or modifications do not include spontaneous mutations that arise naturally (e.g., via aberrant DNA replication).

Several types of mutations or modifications are known in the art. In an aspect, a mutation or modification comprises an insertion. An “insertion” refers to the addition of one or more nucleotides or amino acids to a given polynucleotide or amino acid sequence, respectively, as compared to an endogenous reference polynucleotide or amino acid sequence.

In an aspect, a mutation or modification comprises a deletion. A “deletion” refers to the removal of one or more nucleotides or amino acids to a given polynucleotide or amino acid sequence, respectively, as compared to an endogenous reference polynucleotide or amino acid sequence.

In an aspect, a mutation or modification comprises a substitution. A “substitution” refers to the replacement of one or more nucleotides or amino acids to a given polynucleotide or amino acid sequence, respectively, as compared to an endogenous reference polynucleotide or amino acid sequence. In an aspect, a “substitution allele” refers to a nucleic acid sequence at a particular locus comprising a substitution.

In an aspect, a mutation or modification comprises an inversion. An “inversion” refers to when a segment of a polynucleotide or amino acid sequence is reversed end-to-end. In an aspect, a mutation or modification provided herein comprises a mutation selected from the group consisting of an insertion, a deletion, a substitution, and an inversion.

In an aspect, a mutation or modification comprises one or more mutation types selected from the group consisting of a nonsense mutation, a missense mutation, a frameshift mutation, a splice-site mutation, and any combinations thereof. As used herein, a “nonsense mutation” refers to a mutation to a nucleic acid sequence that introduces a premature stop codon to an amino acid sequence by the nucleic acid sequence. As used herein, a “missense mutation” refers to a mutation to a nucleic acid sequence that causes a substitution within the amino acid sequence encoded by the nucleic acid sequence. As used herein, a “frameshift mutation” refers to an insertion or deletion to a nucleic acid sequence that shifts the frame for translating the nucleic acid sequence to an amino acid sequence. A “splice-site mutation” refers to a mutation in a nucleic acid sequence that causes an intron to be retained for protein translation, or, alternatively, for an exon to be excluded from protein translation. Splice-site mutations can cause nonsense, missense, or frameshift mutations.

Mutations or modifications in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a mutation or modification provided herein comprises a null mutation. As used herein, a “null mutation” refers to a mutation that confers a decreased function or complete loss-of-function for a protein encoded by a gene comprising the mutation, or, alternatively, a mutation that confers a decreased function or complete loss-of-function for a small RNA encoded by a genomic locus. A null mutation can cause lack or decrease of mRNA transcript production, small RNA transcript production, protein function, or a combination thereof. As used herein, a “null allele” refers to a nucleic acid sequence at a particular locus where a null mutation has conferred a decreased function or complete loss-of-function to the allele.

In an aspect, a “synonymous edit” or “synonymous substitution” is the substitution of one base for another in an exon of a gene coding for a protein, such that the produced amino acid sequence is not modified. This is possible because the genetic code is “degenerate”, meaning that some amino acids are coded for by more than one three-base-pair codon; since some of the codons for a given amino acid differ by just one base pair from others coding for the same amino acid, a mutation that replaces the “normal” base by one of the alternatives will result in incorporation of the same amino acid into the growing polypeptide chain when the gene is translated.

In an aspect, “codon optimization” refers to experimental approaches designed to improve the codon composition of a recombinant gene based on various criteria without altering the amino acid sequence. This is possible because most amino acids are encoded by more than one codon. Codon optimization may be used to improve gene expression and increase the translation efficiency of a gene of interest by accommodating for codon bias of the host organism.

In an aspect, a mutation or modification provided herein can be positioned in any part of a gene. In an aspect, a mutation or modification provided herein is positioned within an exon of a gene. In an aspect, a mutation or modification provided herein is positioned within an intron of a gene. In a further aspect, a mutation or modification provided herein is positioned within a 5′-untranslated region (UTR) of a gene. In still another aspect, a mutation or modification provided herein is positioned within a 3′-UTR of a gene. In yet another aspect, a mutation or modification provided herein is positioned within a promoter of a gene. In yet another aspect, a mutation or modification provided herein is positioned within a terminator of a gene.

In an aspect, a mutation or modification in a gene results in a reduced level of expression as compared to the gene lacking the mutation. In an aspect, a mutation or modification in a gene results in an increased level of expression as compared to the gene lacking the mutation.

In a further aspect, a mutation or modification in a gene results in a reduced level of activity by a protein or polypeptide encoded by the gene having the mutation or modification as compared to a protein or polypeptide encoded by the gene lacking the mutation or modification. In a further aspect, a mutation or modification in a gene results in an increased level of activity by a protein or polypeptide encoded by the gene having the mutation or modification as compared to a protein or polypeptide encoded by the gene lacking the mutation or modification.

In an aspect, a mutation or modification in a genomic locus results in a reduced level of expression as compared to the genomic locus lacking the mutation or modification. In an aspect, a mutation or modification in a genomic locus results in an increased level of expression as compared to the genomic locus lacking the mutation or modification. In a further aspect, a mutation or modification in a genomic locus results in a reduced level of activity by a protein or polypeptide encoded by the genomic locus having the mutation or modification as compared to a protein or polypeptide encoded by the genomic locus lacking the mutation or modification. In a further aspect, a mutation or modification in a genomic locus results in an increased level of activity by a protein or polypeptide encoded by the genomic locus having the mutation or modification as compared to a protein or polypeptide encoded by the genomic locus lacking the mutation or modification.

Levels of gene expression are routinely investigated in the art. As non-limiting examples, gene expression can be measured using quantitative reverse transcriptase PCR (qRT-PCR), RNA sequencing, or Northern blots. In an aspect, gene expression is measured using qRT-PCR. In an aspect, gene expression is measured using a Northern blot. In an aspect, gene expression is measured using RNA sequencing.

In an aspect, the present disclosure provides for modifications or mutations in a cell that cause changes in gene or protein expression levels. In an aspect, the changes are less than 1 fold compared to the gene or protein expression levels in a control cell lacking the mutation or modification. In an aspect, the changes are at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the gene or protein expression levels in a control cell lacking the mutation or modification. In an aspect, the changes are about 1 to about 10, about 10 to about 20, about 20 to about 50, about 50 to about 100, about 100 to about 200, about 200 to about 500, or about 500 to about 1000 fold compared to the gene or protein expression levels in a control cell lacking the mutation or modification.

The terms “target genomic DNA sequence”, “target sequence”, or “genomic target locus” and the like refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The terms “transformation”, “transfection” and “transduction” are used interchangeably herein to refer to the process of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In some embodiments, a coding sequence for a nucleic acid-guided nuclease is provided in a vector, referred to as an “engine vector.” In some embodiments, the editing cassette may be provided in a vector, referred to as an “editing vector.” In some embodiments, the coding sequence for the nucleic acid-guided nuclease and the editing cassette are provided in the same vector.

As used herein a “control cell” refers to a Saccharomyces cerevisiae cell comprising a transgene encoding a cellobiohydrolase I (CBHI) enzyme. In an aspect, the transgene encoding the CBHI enzyme comprises a nucleic acid sequence as set forth in SEQ ID NO: 326. In an aspect, the transgene encodes a CBHI enzyme comprising an amino acid sequence as set forth in SEQ ID NO: 27.

### Mutations

In an aspect, the present disclosure provides a Saccharomyces cerevisiae cell comprising a transgene encoding a cellobiohydrolase I (CBHI) enzyme and at least one modification affecting the expression or activity of a protein, where a wildtype version of the protein comprises an amino acid sequence selected from the group consisting of SEQ ID NOs: 1 to 73. In an aspect, the expression or activity of the protein is reduced as compared to a control cell lacking the at least one modification. In aspect, the reduction comprises a change of less than 1 fold. In an aspect, the reduction comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression or activity of the protein in the control cell lacking the at least one modification. In an aspect, the reduction comprises a change of about 1 to about 10, about 10 to about 20, about 20 to about 50, about 50 to about 100, about 100 to about 200, about 200 to about 500, or about 500 to about 1000 fold compared to the expression or activity of the protein in the control cell lacking the at least one modification. In an aspect, the expression or activity of the protein is increased as compared to a control cell lacking the at least one modification. In an aspect, the protein is CBHI. In aspect, the increase comprises a change of less than 1 fold. In an aspect, the increase comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression or activity of the protein in the control cell lacking the at least one modification. In an aspect, the increase comprises a change of about 1 to about 10, about 10 to about 20, about 20 to about 50, about 50 to about 100, about 100 to about 200, about 200 to about 500, or about 500 to about 1000 fold compared to the expression or activity of the protein in the control cell lacking the at least one modification. In an aspect, the expression of a messenger RNA molecule encoding the protein is reduced as compared to a control cell lacking the at least one modification. In an aspect, the reduction of the expression of the messenger RNA molecule comprises a change of less than 1 fold. In an aspect, the reduction comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the messenger RNA molecule in the control cell lacking the at least one modification. In an aspect, the reduction comprises a change of about 1 to about 10, about 10 to about 20, about 20 to about 50, about 50 to about 100, about 100 to about 200, about 200 to about 500, or about 500 to about 1000 fold compared to the expression of the messenger RNA molecule in the control cell lacking the at least one modification. In an aspect, the expression of a messenger RNA molecule encoding the protein is increased as compared to a control cell lacking the at least one modification. In an aspect, the increase of the expression of a messenger RNA comprises a change of less than 1 fold. In an aspect, the increase comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the messenger RNA molecule in the control cell lacking the at least one modification. In an aspect, the increase comprises a change of about 1 to about 10, about 10 to about 20, about 20 to about 50, about 50 to about 100, about 100 to about 200, about 200 to about 500, or about 500 to about 1000 fold compared to the expression of the messenger RNA molecule in the control cell lacking the at least one modification.

In an aspect, the present disclosure provides Saccharomyces cerevisiae cell comprising a transgene encoding a cellobiohydrolase I (CBHI) enzyme and at least one modification affecting the expression or activity of a protein, where a wildtype version of the protein comprises an amino acid sequence selected from the group consisting of SEQ ID NOs: 1 to 73. In an aspect, the at least one modification results in a null allele of a nucleic acid molecule encoding the protein. In an aspect, null allele comprises a premature stop codon as compared to the wildtype version of the protein. In an aspect, the at least one modification comprises at least one amino acid substitution in the protein.

In an aspect, the at least one modification comprises an edit to a promoter region of a nucleic acid molecule encoding the protein. In an aspect, a wildtype version of the promoter region comprises a nucleic acid sequence as set forth in SEQ ID NO: 319 or SEQ ID NO: 321. In an aspect, the promoter region as disclosed herein comprises an Enolase-2 (ENO2) promoter region. In an aspect, the promoter region comprises a promoter region selected from the group consisting of an Enolase-2 promoter, a Yeast Tat-binding Analog 6 (YTA6) promoter, an aldo-keto reductase superfamily protein (YDL124W) promoter, a Suppressor of Marl-1 protein (SUM1) promoter, a Ubiquitin Specific Peptidase 8 (USP8) promoter, a Bromodomain Factor 1 (BDF1) promoter, a NUclear Pore (NUP100) promoter, and any combinations thereof.

In an aspect, the at least one modification comprises an edit to a terminator region of a nucleic acid molecule encoding the protein. In an aspect, a wildtype version of the terminator region comprises a nucleic acid sequence selected from the group consisting of SEQ ID NOs: 311 to 318. In an aspect, the terminator region as disclosed herein comprises a dityrosine-deficient 1 (DIT1) terminator region. In an aspect, the terminator region is selected from a group consisting of a DIT1 terminator, a Repression Factor of Middle sporulation element (RFM1) terminator, a YHR182W terminator, a Multicopy suppressor of Ers1 Hygromycin B sensitivity (MEH1) terminator, a YBR242W terminator, a Putative serine/Threonine protein Kinase (PTK2) terminator, a YLR406C-A terminator, a Suppressor of ToM1 (STM1) terminator, a glutathione (GSH1) terminator, and any combinations thereof.

In an aspect, the least one modification comprises an insertion of at least one nucleotide in a nucleic acid molecule encoding the protein. In an aspect, the at least one modification comprises a deletion of at least one nucleotide in a nucleic acid molecule encoding the protein.

In an aspect, the present disclosure provides Saccharomyces cerevisiae cell comprising a transgene encoding a cellobiohydrolase I (CBHI) enzyme and at least one modification affecting the expression or activity of a protein, where a wildtype version of the protein comprises an amino acid sequence selected from the group consisting of SEQ ID NOs: 1 to 73. In an aspect, a nucleic acid molecule comprising the at least one modification comprises a nucleic acid sequence selected from the group consisting of SEQ ID NOs: 74 to 281.

In an aspect, the present disclosure provides a Saccharomyces cerevisiae cell comprising a transgene encoding a cellobiohydrolase I (CBHI) enzyme and at least one modification affecting the expression or activity of a protein, where a wildtype version of the protein comprises an amino acid sequence selected from the group consisting of SEQ ID NOs: 1 to 73. In an aspect, the S. cerevisiae cell exhibits enhanced CBHI activity as compared to a control cell lacking the at least one modification.

In an aspect, the present disclosure provides a Saccharomyces cerevisiae cell comprising a transgene encoding a cellobiohydrolase I (CBHI) enzyme and a null allele of a nucleic acid molecule encoding a protein, wherein a wildtype version of the protein comprises an amino acid sequence selected from the group consisting of SEQ ID NOs: 1 to 73. In an aspect, expression of the protein is reduced as compared to a control cell lacking the null allele. In an aspect, the expression of a messenger RNA encoding the protein is reduced as compared to a control cell lacking the null allele. In an aspect, the reduced expression of the protein or the reduced expression of the messenger RNA encoding the protein comprises a change of less than 1 fold compared to the expression of the protein or the messenger RNA in the control cell lacking the null allele. In an aspect, the reduced expression of the protein or the reduced expression of the messenger RNA encoding the protein comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the protein or the messenger RNA in the control cell lacking the null allele. In an aspect, the reduced expression of the protein or the reduced expression of the messenger RNA encoding the protein comprises a change of about 1 to about 10, about 10 to about 20, about 20 to about 50, about 50 to about 100, about 100 to about 200, about 200 to about 500, or about 500 to about 1000 fold compared to the expression of the protein or the messenger RNA in the control cell lacking the null allele. In an aspect, the null allele comprises a premature stop codon as compared to the wildtype version of the protein. In an aspect, the null allele comprises a nucleic acid sequence selected from the group consisting of SEQ ID NOs: 74 to 99, 135 to 141, 150 to 203, 239 to 245, and 254 to 281. In an aspect the S. cerevisiae cell exhibits enhanced CBHI activity as compared to a control cell lacking the null allele.

In an aspect, the present disclosure provides Saccharomyces cerevisiae cell comprising a transgene encoding a cellobiohydrolase I (CBHI) enzyme and at least one substitution allele of a nucleic acid molecule encoding a protein, wherein a wildtype version of the protein comprises an amino acid sequence selected from the group consisting of SEQ ID NOs: 1 to 73. In an aspect the expression of the protein is reduced as compared to a control cell lacking the at least one substitution allele. In an aspect the expression of a messenger RNA encoding the protein is reduced as compared to a control cell lacking the at least one substitution allele. In an aspect the expression of the protein is increased as compared to a control cell lacking the at least one substitution allele. In an aspect, the expression of a messenger RNA encoding the protein is increased as compared to a control cell lacking the at least one substitution allele. In an aspect, the reduced or increased expression of the protein or the reduced or increased expression of the messenger RNA encoding the protein comprises a change of less than 1 fold compared to the expression of the protein or the messenger RNA in the control cell lacking the at least one substitution allele. In an aspect, the reduced or increased expression of the protein or the reduced or increased expression of the messenger RNA encoding the protein comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the protein or the messenger RNA in the control cell lacking the at least one substitution allele. In an aspect, the reduced or increased expression of the protein or the reduced or increased expression of the messenger RNA encoding the protein comprises a change of about 1 to about 10, about 10 to about 20, about 20 to about 50, about 50 to about 100, about 100 to about 200, about 200 to about 500, or about 500 to about 1000 fold compared to the expression of the protein or the messenger RNA in the control cell lacking the at least one substitution allele. In an aspect, the at least one substitution allele comprises a nucleic acid sequence selected from the group consisting of SEQ ID NOs: 100 and 204. In an aspect the S. cerevisiae cell exhibits enhanced CBHI activity as compared to a control cell lacking the at least one substitution allele.

In an aspect, the present disclosure provides a Saccharomyces cerevisiae cell comprising a transgene encoding a cellobiohydrolase I (CBHI) enzyme and at least one synonymous edit of a nucleic acid molecule encoding a protein, wherein a wildtype version of the protein comprises an amino acid sequence selected from the group consisting of SEQ ID NOs: 1 to 73. In an aspect, expression of the protein is reduced as compared to a control cell lacking the synonymous edit. In an aspect, expression of a messenger RNA encoding the protein is reduced as compared to a control cell lacking the synonymous edit. In an aspect, expression of the protein is increased as compared to a control cell lacking the synonymous edit. In an aspect, expression of a messenger RNA encoding the protein is increased as compared to a control cell lacking the synonymous edit. In an aspect, the reduced or increased expression of the protein or the reduced or increased expression of the messenger RNA encoding the protein comprises a change of less than 1 fold compared to the expression of the protein or the messenger RNA in the control cell lacking the synonymous edit. In an aspect, the reduced or increased expression of the protein or the reduced or increased expression of the messenger RNA encoding the protein comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the protein or the messenger RNA in the control cell lacking the synonymous edit. In an aspect, the reduced or increased expression of the protein or the reduced or increased expression of the messenger RNA encoding the protein comprises a change of about 1 to about 10, about 10 to about 20, about 20 to about 50, about 50 to about 100, about 100 to about 200, about 200 to about 500, or about 500 to about 1000 fold compared to the expression of the protein or the messenger RNA in the control cell lacking the synonymous edit. In an aspect, the at least one synonymous edit comprises a nucleic acid sequence selected from the group consisting of SEQ ID NOs: 106 to 109 and 210 to 213. In an aspect, the S. cerevisiae cell exhibits enhanced CBHI activity as compared to a control cell lacking the at least one synonymous edit.

In an aspect, the present disclosure provides a Saccharomyces cerevisiae cell comprising a transgene encoding a cellobiohydrolase I (CBHI) enzyme and at least one regulatory element modification in a nucleic acid molecule encoding a protein, wherein a wildtype version of the protein comprises an amino acid sequence selected from the group consisting of SEQ ID NOs: 1 to 73. In an aspect, the at least one regulatory element modification is within a promoter. In an aspect, a wildtype version of the promoter comprises a nucleic acid sequence as set forth in SEQ ID NO: 319 or SEQ ID NO: 321. In an aspect, at least one regulatory element modification is within a terminator. In an aspect, a wildtype version of the terminator comprises a nucleic acid sequence selected from the group consisting of SEQ ID NOs: 311 to 318. In an aspect, expression of the protein is reduced as compared to a control cell lacking the at least one regulatory element modification. In an aspect, expression of a messenger RNA encoding the protein is reduced as compared to a control cell lacking the at least one regulatory element modification. In an aspect, expression of the protein is increased as compared to a control cell lacking the at least one regulatory element modification. In an aspect, expression of a messenger RNA encoding the protein is increased as compared to a control cell lacking the at least one regulatory element modification. In an aspect, the reduced or increased expression of the protein or the reduced or increased expression of the messenger RNA encoding the protein comprises a change of less than 1 fold compared to the expression of the protein or the messenger RNA in the control cell lacking the at least one regulatory element modification. In an aspect, the reduced or increased expression of the protein or the reduced or increased expression of the messenger RNA encoding the protein comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the protein or the messenger RNA in the control cell lacking the at least one regulatory element modification. In an aspect, the reduced or increased expression of the protein or the reduced or increased expression of the messenger RNA encoding the protein comprises a change of about 1 to about 10, about 10 to about 20, about 20 to about 50, about 50 to about 100, about 100 to about 200, about 200 to about 500, or about 500 to about 1000 fold compared to the expression of the protein or the messenger RNA in the control cell lacking the at least one regulatory element modification. In an aspect, the at least one regulatory element modification comprises an insertion or deletion of at least one nucleotide. In an aspect, the least one regulatory element modification comprises a substitution of at least one nucleotide. In an aspect, the at least one regulatory element modification comprises an inversion of at least two nucleotides. In an aspect, the at least one regulatory element modification comprises a nucleic acid sequence selected from the group consisting of SEQ ID NOs: 101 to 105, 110 to 128, 130, 142 to 149, 205 to 209, 214 to 232, 234, and 246 to 253. In an aspect, the S. cerevisiae cell exhibits enhanced CBHI activity as compared to a control cell lacking the at least one regulator element modification.

In an aspect, the present disclosures provides a Saccharomyces cerevisiae cell comprising a transgene encoding a cellobiohydrolase I (CBHI) enzyme and at least one insertion or deletion in a nucleic acid molecule encoding a protein, wherein a wildtype version of the protein comprises an amino acid sequence selected from the group consisting of SEQ ID NOs: 1 to 73. In an aspect, the expression of the protein is reduced as compared to a control cell lacking the at least one insertion or deletion. In an aspect, expression of a messenger RNA encoding the protein is reduced as compared to a control cell lacking the at least one insertion or deletion. In an aspect, expression of the protein is increased as compared to a control cell lacking the at least one insertion or deletion. In an aspect, expression of a messenger RNA encoding the protein is increased as compared to a control cell lacking the at least one insertion or deletion. In an aspect, the reduced or increased expression of the protein or the reduced or increased expression of the messenger RNA encoding the protein comprises a change of less than 1 fold compared to the expression of the protein or the messenger RNA in the control cell lacking the at least one insertion or deletion. In an aspect, the reduced or increased expression of the protein or the reduced or increased expression of the messenger RNA encoding the protein comprises a change of at least about 1 fold, at least about 2 fold, at least about 3 fold, at least about 4 fold, at least about 5 fold, at least about 6 fold, at least about 7 fold, at least about 8 fold, at least about 9 fold, at least about 10 fold, at least 15 fold, at least 20 fold, at least 30 fold, at least 40 fold, or at least 50 fold compared to the expression of the protein or the messenger RNA in the control cell lacking the at least one insertion or deletion. In an aspect, the reduced or increased expression of the protein or the reduced or increased expression of the messenger RNA encoding the protein comprises a change of about 1 to about 10, about 10 to about 20, about 20 to about 50, about 50 to about 100, about 100 to about 200, about 200 to about 500, or about 500 to about 1000 fold compared to the expression of the protein or the messenger RNA in the control cell lacking the at least one insertion or deletion. In an aspect, the at least one insertion or deletion is an insertion. In an aspect, the insertion comprises the insertion of at least one nucleotide. In an aspect, the at least one insertion or deletion is a deletion. In an aspect, the deletion comprises the deletion of at least one nucleotide. In an aspect, the at least one insertion or deletion is positioned within a region of the nucleic acid molecule selected from the group consisting of a promoter region, a 5′ untranslated region (UTR), an exon, an intron, a terminator region, and a 3′ UTR. In an aspect, the S. cerevisiae cell exhibits enhanced CBHI activity as compared to a control cell lacking the at least one at least one insertion or deletion.

In an aspect, the present disclosure provides a Saccharomyces cerevisiae cell comprising a transgene encoding a cellobiohydrolase I (CBHI) enzyme, a first modification affecting the expression or activity of a first protein, and a second modification affecting the expression or activity of a second protein, wherein a wildtype version of the first protein and a wildtype version of the second protein each comprise an amino acid sequence selected from the group consisting of SEQ ID NOs: 1 to 73. In an aspect, the cell further comprises a third modification affecting the expression or activity of a third protein, wherein a wildtype version of the third protein comprises an amino acid sequence selected from the group consisting of SEQ ID NOs: 1 to 73. In an aspect, the cell further comprises a fourth modification affecting the expression or activity of a fourth protein, wherein a wildtype version of the fourth protein comprises an amino acid sequence selected from the group consisting of SEQ ID NOs: 1 to 73. In an aspect, the cell further comprises a fifth, a sixth, an eight, a ninth, or a tenth modification affecting the expression or activity of a fifth, a sixth, an eight, a ninth, or a tenth protein, wherein a wildtype version of the fifth, a sixth, an eight, a ninth, or a tenth protein comprises an amino acid sequence selected from the group consisting of SEQ ID NOs: 1 to 73. In an aspect, the cell comprises at least 1, at least 2, at least 3, at least 4, at least 5, at least 6, at least 7, at least 8, at least 9, at least 10 modifications affecting the expression or activity of at least 1, at least 2, at least 3, at least 4, at least 5, at least 6, at least 7, at least 8, at least 9, at least 10 proteins, wherein a wildtype version of the at least 1, at least 2, at least 3, at least 4, at least 5, at least 6, at least 7, at least 8, at least 9, at least 10 proteins comprises an amino acid sequence selected from the group consisting of SEQ ID NOs: 1 to 73. In an aspect, the cell comprises 1, 2, 3, 4, 5, 6, 7, 8, 9, or 10 modifications affecting the expression or activity of 1, 2, 3, 4, 5, 6, 7, 8, 9, or 10 proteins, wherein a wildtype version of the 1, 2, 3, 4, 5, 6, 7, 8, 9, or 10 proteins comprises an amino acid sequence selected from the group consisting of SEQ ID NOs: 1 to 73. In an aspect, the cell comprises 1 to 5, 5 to 10, 10 to 20, 20 to 50, or 50 to 100 modifications affecting the expression or activity of 1 to 5, 5 to 10, 10 to 20, 20 to 50, or 50 to 100 proteins, wherein a wildtype version of the 1 to 5, 5 to 10, 10 to 20, 20 to 50, or 50 to 100 proteins comprises an amino acid sequence selected from the group consisting of SEQ ID NOs: 1 to 73. In an aspect, expression or activity of the first protein, the second protein, or both, is reduced as compared to a control cell lacking the at least one modification.

In an aspect, expression or activity of the first protein, the second protein, or both is increased as compared to a control cell lacking the at least one modification. In an aspect, expression of a messenger RNA (mRNA) molecule encoding the first protein, an mRNA molecule encoding the second protein, or both, is reduced as compared to a control cell lacking the at least one modification. In an aspect, expression of a messenger RNA (mRNA) molecule encoding the first protein, an mRNA molecule encoding the second protein, or both, is increased as compared to a control cell lacking the at least one modification. In an aspect, (a) the first modification results in a null allele of a nucleic acid molecule encoding the first protein; (b) the second modification results in a null allele of a nucleic acid molecule encoding the second protein; or (c) both (a) and (b). In an aspect, (a) the null allele of a nucleic acid molecule encoding the first protein comprises a premature stop codon as compared to the wildtype version of the first protein; (b) the null allele of a nucleic acid molecule encoding the second protein comprises a premature stop codon as compared to the wildtype version of the second protein; or both (a) and (b). In an aspect, (a) the first modification comprises at least one amino acid substitution in the first protein; (b) the second modification comprises at least one amino acid substitution in the second protein; or (c) both (a) and (b). In an aspect, (a) the first modification comprises an edit to a promoter region of a nucleic acid molecule encoding the first protein; (b) the second modification comprises an edit to a promoter region of a nucleic acid molecule encoding the second protein; or (c) both (a) and (b). In an aspect, a wildtype version of the promoter region of the nucleic acid molecule encoding the first protein comprises a nucleic acid sequence as set forth in SEQ ID NO: 319 or SEQ ID NO: 321, and wherein a wildtype version of the promoter region of the nucleic acid molecule encoding the second protein comprises a nucleic acid sequence as set forth in SEQ ID NO: 319 or SEQ ID NO: 321. In an aspect, (a) the first modification comprises an edit to a terminator region of a nucleic acid molecule encoding the first protein; (b) the second modification comprises an edit to a terminator region of a nucleic acid molecule encoding the second protein; or (c) both (a) and (b). In an aspect, a wildtype version of the terminator region of the nucleic acid molecule encoding the first protein comprises a nucleic acid sequence selected from the group consisting of SEQ ID NOs: 311 to 318, and wherein a wildtype version of the terminator region of the nucleic acid molecule encoding the second protein comprises a nucleic acid sequence selected from the group consisting of SEQ ID NOs: 311 to 318. In an aspect, (a) the first modification comprises an insertion of at least one nucleotide in a nucleic acid molecule encoding the first protein; (b) the second modification comprises an insertion of at least one nucleotide in a nucleic acid molecule encoding the second protein; or (c) both (a) and (b). In an aspect, (a) the first modification comprises a deletion of at least one nucleotide in a nucleic acid molecule encoding the first protein; (b) the second modification comprises a deletion of at least one nucleotide in a nucleic acid molecule encoding the second protein; or (c) both (a) and (b). In an aspect, the S. cerevisiae cell exhibits enhanced CBHI activity as compared to a control cell lacking the first modification and the second modification. In an aspect, the transgene comprises a promoter operably linked to a nucleic acid sequence encoding the CBHI enzyme. In an aspect, the promoter comprises SEQ ID NO: 319. In an aspect, transgene comprises a terminator operably linked to a nucleic acid sequence encoding the CBHI enzyme. In an aspect, the transgene is codon optimized for S. cerevisiae. In an aspect, the transgene encodes a polypeptide comprising SEQ ID NO: 2774.

In an aspect, the present disclosure provides a Saccharomyces cerevisiae cell comprising a transgene encoding a cellobiohydrolase I (CBHI) enzyme comprising a nucleic acid sequence having at least 40%, at least 45%, at least 50%, at least 55%, at least 60%, at least 65%, at least 70%, at least 75%, at least 80%, at least 85%, at least 90%, at least 95%, or at least 99% identity to SEQ ID NO: 326 or a complement thereof. In an aspect, the present disclosure provides a Saccharomyces cerevisiae cell comprising a transgene encoding a cellobiohydrolase I (CBHI) enzyme comprising an amino acid sequence having at least 40%, at least 45%, at least 50%, at least 55%, at least 60%, at least 65%, at least 70%, at least 75%, at least 80%, at least 85%, at least 90%, at least 95%, or at least a 99% identity to SEQ ID NO: 27.

### Nucleic Acid-Guided Nuclease and Nickase Editing Generally

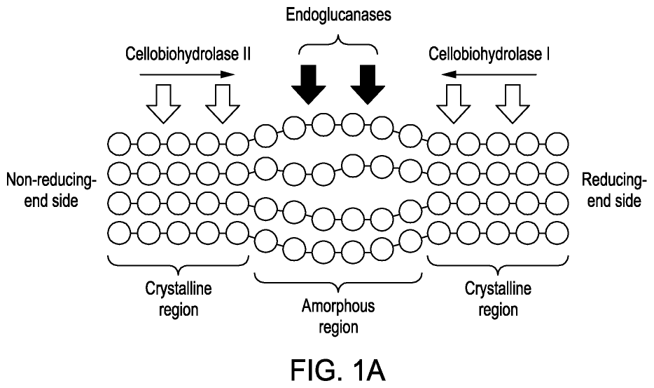

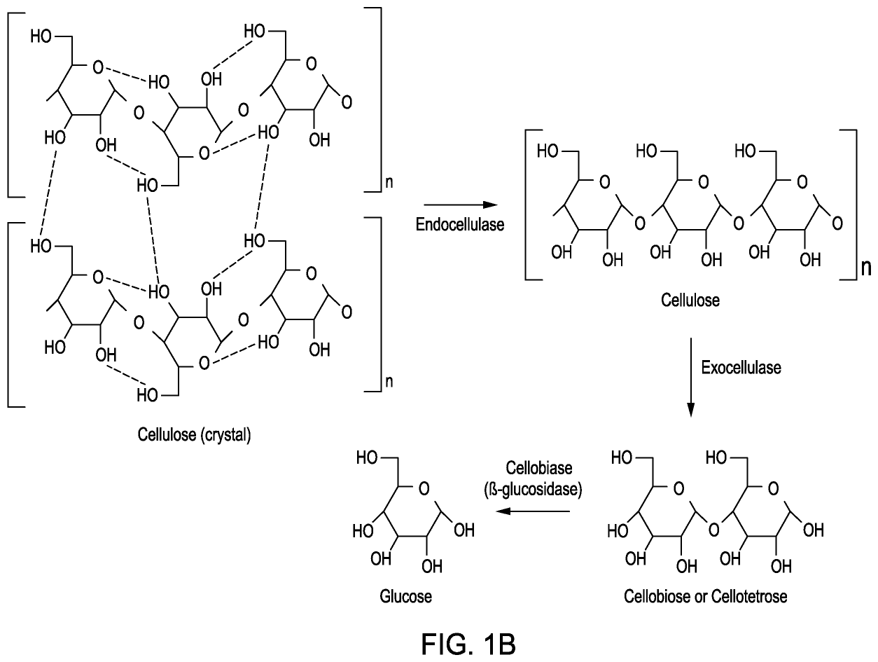

Cellobiohydrolase I (CBH1) is an enzyme involved in the degradation of cellulose. The enzyme functions as an exo-cellulase that releases cellobiose units from the reducing-end of a cellulose chain. CBH1 along with a cocktail of other enzymes (see FIGS. 1A and 1B, described infra) can ultimately convert cellulose to glucose. The CBH1 enzyme has significance in a consolidated bioprocessing (CBP) system. Improvements in the expression of CBH1 in yeast is a first step in the development of better CBP strains. The present disclosure provides amino acid and nucleic acid variants of a strain of S. cerevisiae that has been rationally engineered to express CBHI, via a nucleic acid-guided nuclease (i.e., CRISPR enzyme) in a closed automated system.

Generally, a nucleic acid-guided nuclease or nickase fusion enzyme complexed with an appropriate synthetic guide nucleic acid in a cell can cut the genome of the cell at a desired location. The guide nucleic acid helps the nucleic acid-guided nuclease or nickase fusion enzyme recognize and cut the DNA at a specific target sequence. By manipulating the nucleotide sequence of the guide nucleic acid, the nucleic acid-guided nuclease or nickase fusion enzyme may be programmed to target any DNA sequence for cleavage as long as an appropriate protospacer adjacent motif (PAM) is nearby. In certain aspects, the nucleic acid-guided nuclease system or nucleic acid-guided nickase fusion editing system (i.e., CF editing system) may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease or nickase fusion enzyme.

In general, a guide nucleic acid (e.g., gRNA) complexes with a compatible nucleic acid-guided nuclease or nickase fusion enzyme and can then hybridize with a target sequence, thereby directing the nuclease or nickase fusion to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. Preferably and typically, the guide nucleic acid comprises RNA and the gRNA is encoded by a DNA sequence on an editing cassette along with the coding sequence for a repair template. Covalently linking the gRNA and repair template allows one to scale up the number of edits that can be made in a population of cells tremendously. Methods and compositions for designing and synthesizing editing cassettes (e.g., CREATE cassettes) are described in U.S. Pat. Nos. 10,240,167; 10,266,849; 9,982,278; 10,351,877; 10,364,442; 10,435,715; 10,669,559; 10,711,284; and 10,731,180, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease or nickase fusion enzyme to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease or gRNA/nickase fusion complex binds to a target sequence as determined by the guide RNA, and the nuclease or nickase fusion recognizes a protospacer adjacent motif (PAM) sequence adjacent to the target sequence. The target sequence can be any polynucleotide endogenous or exogenous to the cell, or in vitro. For example, in the case of mammalian cells the target sequence is typically a polynucleotide residing in the nucleus of the cell. A target sequence can be a sequence encoding a gene product (e.g., a protein) or a noncoding sequence (e.g., a regulatory polynucleotide, an intron, a PAM, a control sequence, or “junk” DNA). The proto-spacer mutation (PAM) is a short nucleotide sequence recognized by the gRNA/nuclease complex. The precise preferred PAM sequence and length requirements for different nucleic acid-guided nucleases or nickase fusions vary; however, PAMs typically are 2-10 base-pair sequences adjacent or in proximity to the target sequence and, depending on the nuclease or nickase, can be 5′ or 3′ to the target sequence.

In most embodiments, genome editing of a cellular target sequence both introduces a desired DNA change (i.e., the desired edit) to a cellular target sequence, e.g., the genomic DNA of a cell, and removes, mutates, or renders inactive a proto-spacer/spacer mutation (PAM) region in the cellular target sequence (e.g., thereby rendering the target site immune to further nuclease binding). Rendering the PAM and/or spacer at the cellular target sequence inactive precludes additional editing of the cell genome at that cellular target sequence, e.g., upon subsequent exposure to a nucleic acid-guided nuclease or nickase fusion complexed with a synthetic guide nucleic acid in later rounds of editing. Thus, cells having the desired cellular target sequence edit and an altered PAM or spacer can be selected for by using a nucleic acid-guided nuclease or nickase fusion complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM or spacer alteration will not be cut, as these edited cells no longer contain the necessary PAM site and will continue to grow and propagate.

As for the nuclease or nickase fusion component of the nucleic acid-guided nuclease editing system, a polynucleotide sequence encoding the nucleic acid-guided nuclease or nickase fusion enzyme can be codon optimized for expression in particular cell types, such as bacterial, yeast, and, here, mammalian cells. The choice of the nucleic acid-guided nuclease or nickase fusion enzyme to be employed depends on many factors, such as what type of edit is to be made in the target sequence and whether an appropriate PAM is located close to the desired target sequence. Nucleic acid-guided nucleases (i.e., CRISPR enzymes) of use in the methods described herein include but are not limited to Cas 9, Cas 12/CpfI, MAD2, or MAD7, MAD 2007 or other MADzymes and MADzyme systems (see U.S. Pat. Nos. 9,982,279; 10,337,028; 10,435,714; 10,011,849; 10,626,416; 10,604,746; 10,665,114; 10,640,754; 10,876,102; 10,883,077; 10,704,033; 10,745,678; 10,724,021; 10,767,169; and 10,870,761 for sequences and other details related to engineered and naturally-occurring MADzymes). Nickase fusion enzymes typically comprise a CRISPR nucleic acid-guided nuclease engineered to cut one DNA strand in a target DNA rather than making a double-stranded cut, and the nickase portion is fused to a reverse transcriptase. For more information on nickases and nickase fusion editing see U.S. Pat. No. 10,689,669 and U.S. Ser. No. 16/740,418 (U.S. Pat. No. 10,689,669); Ser. No. 16/740,420 (U.S. Publication No. US2021/0214671 A1) and Ser. No. 16/740,421, all of which were filed 11 Jan. 2020. A coding sequence for a desired nuclease or nickase fusion may be on an “engine vector” along with other desired sequences such as a selective marker or may be transfected into a cell as a protein or ribonucleoprotein (“RNP”) complex. Any references cited herein, including, e.g., all patents, published patent applications, and non-patent publications, are incorporated herein by reference in their entirety.

Another component of the nucleic acid-guided nuclease or nickase fusion system is the repair template comprising homology to the cellular target sequence. In some exemplary embodiments, the repair template is in the same editing cassette as (e.g., is covalently-linked to) the guide nucleic acid and typically is under the control of the same promoter as the gRNA (that is, a single promoter driving the transcription of both the editing gRNA and the repair template). The repair template is designed to serve as a template for homologous recombination with a cellular target sequence cleaved by a nucleic acid-guided nuclease or serve as the template for template-directed repair via a nickase fusion, as a part of the gRNA/nuclease complex. A repair template polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Pat. No. 10,711,284, incorporated by reference herein.

In certain preferred aspects, the repair template can be provided as an oligonucleotide of between 20-300 nucleotides, more preferably between 50-250 nucleotides. As described infra, the repair template comprises a region that is complementary to a portion of the cellular target sequence. When optimally aligned, the repair template overlaps with (is complementary to) the cellular target sequence by, e.g., about as few as 4 (in the case of nickase fusions) and as many as 20, 25, 30, 35, 40, 50, 60, 70, 80, 90 or more nucleotides (in the case of nucleases). The repair template comprises a region complementary to the cellular target sequence flanking the edit locus or difference between the repair template and the cellular target sequence. The desired edit may comprise an insertion, deletion, modification, or any combination thereof compared to the cellular target sequence.

As described in relation to the gRNA, the repair template may be provided as part of a rationally-designed editing cassette along with a promoter to drive transcription of both the gRNA and repair template. As described below, the editing cassette may be provided as a linear editing cassette, or the editing cassette may be inserted into an editing vector. Moreover, there may be more than one, e.g., two, three, four, or more editing gRNA/repair template pairs rationally-designed editing cassettes linked to one another in a linear “compound cassette” or inserted into an editing vector; alternatively, a single rationally-designed editing cassette may comprise two to several editing gRNA/repair template pairs, where each editing gRNA is under the control of separate different promoters, separate promoters, or where all gRNAs/repair template pairs are under the control of a single promoter. In some embodiments the promoter driving transcription of the editing gRNA and the repair template (or driving more than one editing gRNA/repair template pair) is an inducible promoter. In many if not most embodiments of the compositions, methods, modules and instruments described herein, the editing cassettes make up a collection or library editing of gRNAs and of repair templates representing, e.g., gene-wide or genome-wide libraries of editing gRNAs and repair templates.

In addition to the repair template, the editing cassettes comprise one or more primer binding sites to allow for PCR amplification of the editing cassettes. The primer binding sites are used to amplify the editing cassette by using oligonucleotide primers, and may be biotinylated or otherwise labeled. In addition, the editing cassette may comprise a barcode. A barcode is a unique DNA sequence that corresponds to the repair template sequence such that the barcode serves as a proxy to identify the edit made to the corresponding cellular target sequence. The barcode typically comprises four or more nucleotides. Also, in preferred embodiments, an editing cassette or editing vector or engine vector further comprises one or more nuclear localization sequences (NLSs), such as about or more than about 1, 2, 3, 4, 5, 6, 7, 8, 9, 10, or more NLSs.

Nucleic Acid-Guided Nuclease-Directed Genome Editing of S. cerevisiae

Cellobiohydrolase I (CBH1) is an enzyme involved in the degradation of cellulose. CBH1 along with a cocktail of other enzymes (see FIGS. 1A and 1B, described below) can ultimately convert cellulose to glucose. The CBH1 enzyme has significance in a consolidated bioprocessing (CBP) system. CBP combines multiple biological steps into a single reaction system. In this process, a microbe expresses a set of enzymes used to degrade an input feedstock (usually a waste plant material), ultimately converting it to soluble sugars, which are fermented by the microbe to produce a fuel (such as ethanol) or commercially-relevant chemical. The expression of the enzymes, hydrolysis of the feedstock, and fermentation of the sugars to a fuel/chemical are consolidated into a single step which has cost saving advantages compared to more complex multistep processes. CBH1 is one of the key enzymes necessary for the efficient degradation of cellulosic feedstocks. Improvements in the expression of a CBH1 in yeast is a first step in the development of better CBP strains.

FIG. 1A is a simplified depiction of the breakdown of cellulose. There are two main regions found in cellulose fibers—the crystalline and amorphous regions. The crystalline regions have a high order organization of microfibrils, while a region of less microfibril order is called amorphous. The amorphous region results from the breakage and disorder of hydrogen bonds. There are three types of enzymes that break down cellulose: Cellobiohydrolase I is an exocellulase that cleaves two to four cellobiose units from the reducing-end of a cellulose chain; cellobiohydrolase II is an exocellulase that cleaves two to four cellobiose units from the non-reducing-end of the cellulose chain; and endocellulases or endoglucanases randomly cleave internal bonds at amorphous sites to create new cellulose chain ends, which are then available for hydrolysis for cellobiohydrolase I and cellobiohydrolase II. The circles in this FIG. 1A are glucose residues.

FIG. 1B depicts more detail of the process shown in FIG. 1A. Again, there are three types of reaction catalyzed by cellulases: Breakage of noncovalent interactions present in the amorphous structure of cellulose catalyzed by endocellulase; 2. Hydrolysis of chain ends to break the polymer into smaller sugars catalyzed by the exocellulases cellobiohydrolase I and cellobiohydrolase II; and 3. Hydrolysis of disaccharides and tetrasaccharides into glucose catalyzed by beta-glucosidase.

Automated Cell Editing Instrument and Modules to Perform Nucleic Acid-Guided Nuclease Editing in S. cerevisiae Cells

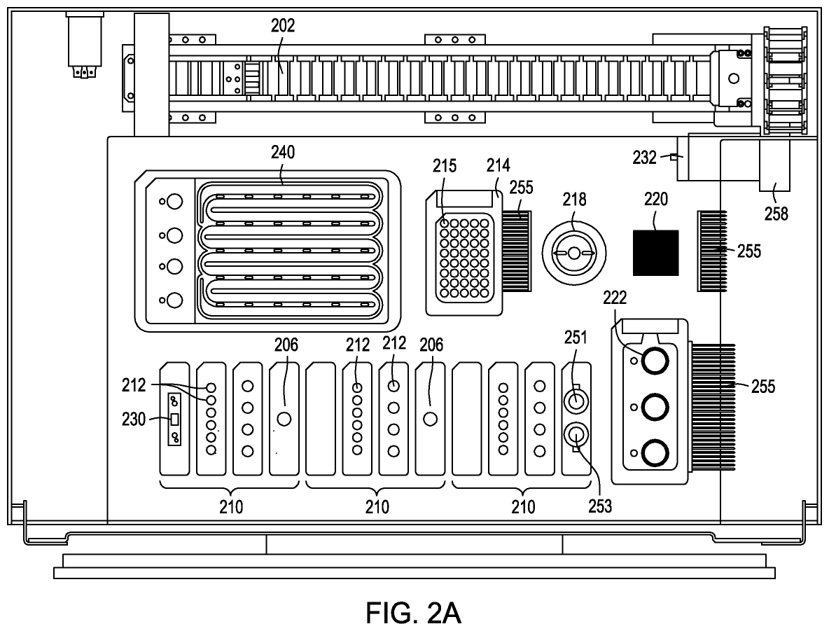

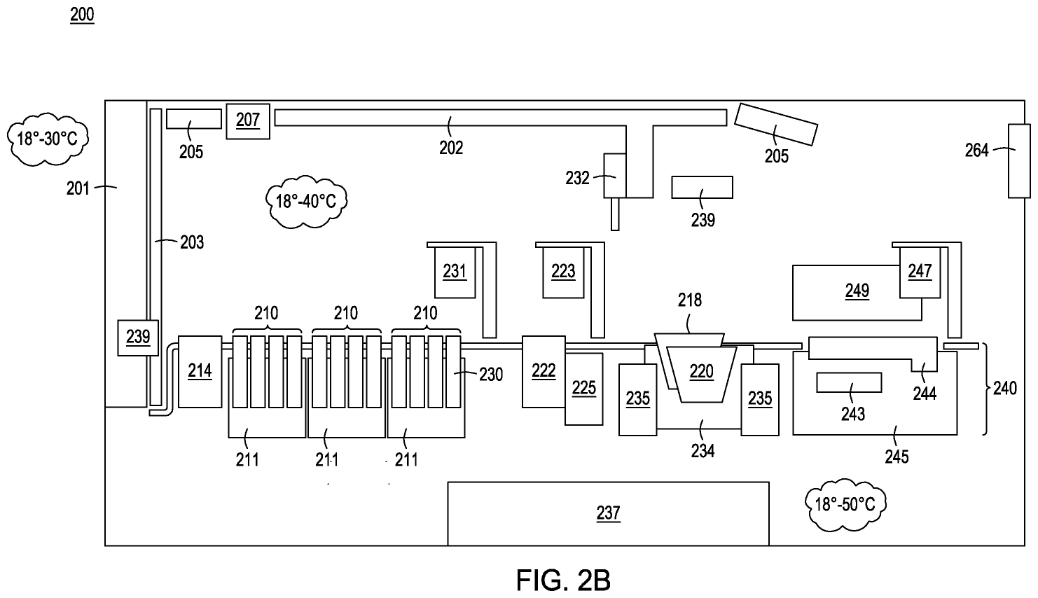

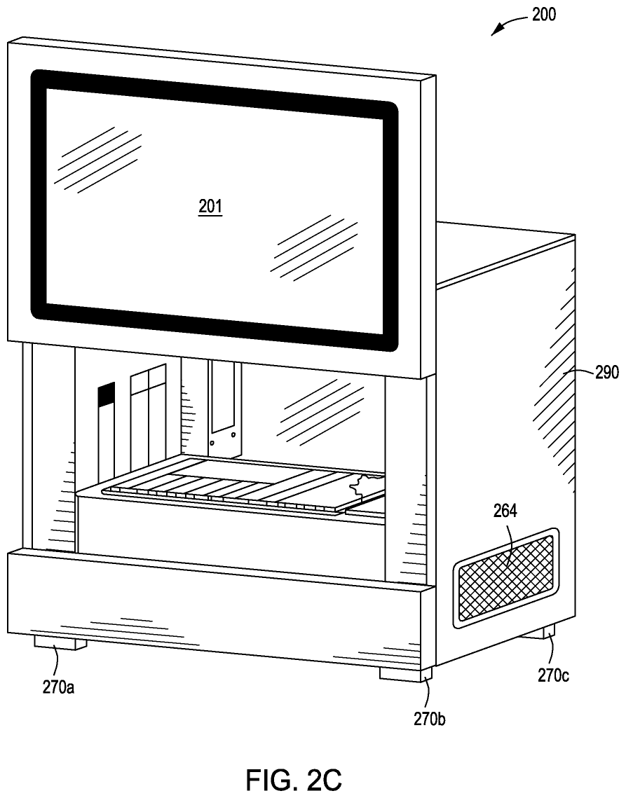

FIG. 2A depicts an exemplary automated multi-module cell processing instrument 200 to, e.g., perform targeted gene editing of live cells. The instrument 200, for example, may be and preferably is designed as a stand-alone benchtop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved.

Also included in the automated multi-module cell processing instrument 200 are reagent cartridges 210 (see, U.S. Pat. No. 10,376,889; 10,406,525; 10,478,822; 10,576,474; 10,639,637; 10,738,271; and 10,799,868) comprising reservoirs 212 and transformation module 230 (e.g., a flow-through electroporation device as described in U.S. Pat. No. 10,435,713; 10,443,074; and 10,851,389), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge and wash cartridge may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV (see, e.g., WO2018015544A1), or Beckman Coulter, Inc. of Fort Collins, CO. (see, e.g., US20160018427A1). Pipette tips 215 may be provided in a pipette transfer tip supply 214 for use with the air displacement pipettor 232. The robotic liquid handling system allows for the transfer of liquids between modules without human intervention.

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (for details, see U.S. Pat. No. 10,435,662; 10,433,031; 10,590,375; 10,717,959; and 10,883,095). Additionally seen is a tangential flow filtration (TFF) module 222 (for details, see U.S. Ser. Nos. 16/516,701 and 16/798,302). Also illustrated as part of the automated multi-module cell processing instrument 200 of FIG. 2A is a singulation module 240 (e.g., a solid wall isolation, incubation and normalization device (SWIIN device) is shown here and described in detail in U.S. Pat. No. 10,533,152; 10,633,626; 10,633,627; 10,647,958; 10,723,995; 10,801,008; 10,851,339; 10,954,485; 10,532,324; 10,625,212; 10,774,462; and 10,835,869), served by, e.g., robotic liquid handing system 258 and air displacement pipettor 232. Additionally seen is a selection module 220 which may employ magnet separation. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink (not shown) such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different reagents in different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 223. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. A selection module is seen at 220. Also seen is the SWIIN module 240, comprising a SWIIN cartridge 244, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one camera on either side of multi-module cell processing instrument 200). Finally, element 237 comprises electronics, such as a processor, circuit control boards, high-voltage amplifiers, power supplies, and power entry; as well as pneumatics, such as pumps, valves and sensors.

FIG. 2C illustrates a front perspective view of multi-module cell processing instrument 200 for use in as a benchtop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. Nos. 10,253,316; 10,329,559; 10,323,242; 10,421,959; 10,465,185; 10,519,437; 10,584,333; 10,584,334; 10,647,982; 10,689,645; 10,738,301; 10,738,663; 10,947,532; 10,894,958; 10,954,512; and 11,034,953, all of which are herein incorporated by reference in their entirety.

The following exemplary, non-limiting, embodiments are envisioned:
1. A Saccharomyces cerevisiae cell comprising a transgene encoding a cellobiohydrolase I (CBHI) enzyme and at least one modification affecting the expression or activity of a protein, wherein a wildtype version of the protein comprises an amino acid sequence selected from the group consisting of SEQ ID NOs: 1 to 73.
2. The S. cerevisiae cell of embodiment 1, wherein the expression or activity of the protein is reduced as compared to a control cell lacking the at least one modification.
3. The S. cerevisiae cell of embodiment 1, wherein the expression or activity of the protein is increased as compared to a control cell lacking the at least one modification.
4. The S. cerevisiae cell of embodiment 1, wherein expression of a messenger RNA molecule encoding the protein is reduced as compared to a control cell lacking the at least one modification.
5. The S. cerevisiae cell of embodiment 1, wherein expression of a messenger RNA molecule encoding the protein is increased as compared to a control cell lacking the at least one modification.
6. The S. cerevisiae cell of any one of embodiments 1 to 5, wherein the at least one modification results in a null allele of a nucleic acid molecule encoding the protein.
7. The S. cerevisiae cell of embodiment 6, wherein the null allele comprises a premature stop codon as compared to the wildtype version of the protein.
8. The S. cerevisiae cell of any one of embodiments 1 to 5, wherein the at least one modification comprises at least one amino acid substitution in the protein.
9. The S. cerevisiae cell of any one of embodiments 1 to 5, wherein the at least one modification comprises an edit to a promoter region of a nucleic acid molecule encoding the protein.
10. The S. cerevisiae cell of any one of embodiments 1 to 5, wherein the at least one modification comprises an edit to a terminator region of a nucleic acid molecule encoding the protein.
11. The S. cerevisiae cell of embodiment 10, wherein a wildtype version of the terminator region comprises a nucleic acid sequence selected from the group consisting of SEQ ID NOs: 311 to 318. 12. The S. cerevisiae cell of any one of embodiments 1 to 11, wherein the at least one modification comprises an insertion of at least one nucleotide in a nucleic acid molecule encoding the protein.
13. The S. cerevisiae cell of any one of embodiments 1 to 11, wherein the at least one modification comprises a deletion of at least one nucleotide in a nucleic acid molecule encoding the protein.
14. The S. cerevisiae cell of embodiment 1, wherein a nucleic acid molecule comprising the at least one modification comprises a nucleic acid sequence selected from the group consisting of SEQ ID NOs: 74 to 281.
15. The S. cerevisiae cell of any one of embodiments 1 to 14, wherein the S. cerevisiae cell exhibits enhanced CBHI activity as compared to a control cell lacking the at least one modification.
16. A Saccharomyces cerevisiae cell comprising a transgene encoding a cellobiohydrolase I (CBHI) enzyme and a null allele of a nucleic acid molecule encoding a protein, wherein a wildtype version of the protein comprises an amino acid sequence selected from the group consisting of SEQ ID NOs: 1 to 73.
17. The S. cerevisiae cell of embodiment 16, wherein expression of the protein is reduced as compared to a control cell lacking the null allele.
18. The S. cerevisiae cell of embodiment 16 or 17, wherein expression of a messenger RNA encoding the protein is reduced as compared to a control cell lacking the null allele.
19. The S. cerevisiae cell of any one of embodiments 16 to 18, wherein the null allele comprises a premature stop codon as compared to the wildtype version of the protein.
20. The S. cerevisiae cell of any one of embodiments 16 to 18, wherein the null allele comprises a nucleic acid sequence selected from the group consisting of SEQ ID NOs: 74 to 99, 135 to 141, 150 to 203, 239 to 245, and 254 to 281.
21. The S. cerevisiae cell of any one of embodiments 16 to 20, wherein the S. cerevisiae cell exhibits enhanced CBHI activity as compared to a control cell lacking the null allele.
22. A Saccharomyces cerevisiae cell comprising a transgene encoding a cellobiohydrolase I (CBHI) enzyme and at least one substitution allele of a nucleic acid molecule encoding a protein, wherein a wildtype version of the protein comprises an amino acid sequence selected from the group consisting of SEQ ID NOs: 1 to 73.
23. The S. cerevisiae cell of embodiment 22, wherein expression of the protein is reduced as compared to a control cell lacking the at least one substitution allele.
24. The S. cerevisiae cell of embodiment 22 or 23, wherein expression of a messenger RNA encoding the protein is reduced as compared to a control cell lacking the at least one substitution allele.
25. The S. cerevisiae cell of embodiment 22, wherein expression of the protein is increased as compared to a control cell lacking the at least one substitution allele.
26. The S. cerevisiae cell of embodiment 22 or 25, wherein expression of a messenger RNA encoding the protein is increased as compared to a control cell lacking the at least one substitution allele.
27. The S. cerevisiae cell of embodiment 22, wherein the at least one substitution allele comprises a nucleic acid sequence selected from the group consisting of SEQ ID NOs: 100 and 204.
28. The S. cerevisiae cell of any one of embodiments 22 to 27, wherein the S. cerevisiae cell exhibits enhanced CBHI activity as compared to a control cell lacking the at least one substitution allele.
29. A Saccharomyces cerevisiae cell comprising a transgene encoding a cellobiohydrolase I (CBHI) enzyme and at least one synonymous edit of a nucleic acid molecule encoding a protein, wherein a wildtype version of the protein comprises an amino acid sequence selected from the group consisting of SEQ ID NOs: 1 to 73.
30. The S. cerevisiae cell of embodiment 29, wherein expression of the protein is reduced as compared to a control cell lacking the synonymous edit.
31. The S. cerevisiae cell of embodiment 29 or 30, wherein expression of a messenger RNA encoding the protein is reduced as compared to a control cell lacking the synonymous edit.
32. The S. cerevisiae cell of embodiment 29, wherein expression of the protein is increased as compared to a control cell lacking the synonymous edit.
33. The S. cerevisiae cell of embodiment 29 or 32, wherein expression of a messenger RNA encoding the protein is increased as compared to a control cell lacking the synonymous edit.
34. The S. cerevisiae cell of embodiment 29, wherein the at least one synonymous edit comprises a nucleic acid sequence selected from the group consisting of SEQ ID NOs: 106 to 109 and 210 to 213.
35. The S. cerevisiae cell of any one of embodiments 29 to 34, wherein the S. cerevisiae cell exhibits enhanced CBHI activity as compared to a control cell lacking the at least one synonymous edit.
36. A Saccharomyces cerevisiae cell comprising a transgene encoding a cellobiohydrolase I (CBHI) enzyme and at least one regulatory element modification in a nucleic acid molecule encoding a protein, wherein a wildtype version of the protein comprises an amino acid sequence selected from the group consisting of SEQ ID NOs: 1 to 73.
37. The S. cerevisiae cell of embodiment 36, wherein the at least one regulatory element modification is within a promoter.
38. The S. cerevisiae cell of embodiment 37, wherein a wildtype version of the promoter comprises a nucleic acid sequence as set forth in SEQ ID NO: 319 or SEQ ID NO: 321.
39. The S. cerevisiae cell of embodiment 36, wherein the at least one regulatory element modification is within a terminator.
40. The S. cerevisiae cell of embodiment 39, wherein a wildtype version of the terminator comprises a nucleic acid sequence selected from the group consisting of SEQ ID NOs: 311 to 318.
41. The S. cerevisiae cell of any one of embodiments 36 to 40, wherein expression of the protein is reduced as compared to a control cell lacking the at least one regulatory element modification.
42. The S. cerevisiae cell of any one of embodiments 36 to 41, wherein expression of a messenger RNA encoding the protein is reduced as compared to a control cell lacking the at least one regulatory element modification.
43. The S. cerevisiae cell of any one of embodiments 36 to 40, wherein expression of the protein is increased as compared to a control cell lacking the at least one regulatory element modification.
44. The S. cerevisiae cell of any one of embodiments 36 to 40 or 43, wherein expression of a messenger RNA encoding the protein is increased as compared to a control cell lacking the at least one regulatory element modification.
45. The S. cerevisiae cell of any one of embodiments 36 to 44, wherein the at least one regulatory element modification comprises an insertion or deletion of at least one nucleotide.
46. The S. cerevisiae cell of any one of embodiments 36 to 44, wherein the at least one regulatory element modification comprises a substitution of at least one nucleotide.
47. The S. cerevisiae cell of any one of embodiments 36 to 44, wherein the at least one regulatory element modification comprises an inversion of at least two nucleotides.
48. The S. cerevisiae cell of embodiment 36, wherein the at least one regulatory element modification comprises a nucleic acid sequence selected from the group consisting of SEQ ID NOs: 101 to 105, 110 to 128, 130, 142 to 149, 205 to 209, 214 to 232, 234, and 246 to 253.
49. The S. cerevisiae cell of any one of embodiments 36 to 48, wherein the S. cerevisiae cell exhibits enhanced CBHI activity as compared to a control cell lacking the at least one regulator element modification.
50. A Saccharomyces cerevisiae cell comprising a transgene encoding a cellobiohydrolase I (CBHI) enzyme and at least one insertion or deletion in a nucleic acid molecule encoding a protein, wherein a wildtype version of the protein comprises an amino acid sequence selected from the group consisting of SEQ ID NOs: 1 to 73.
51. The S. cerevisiae cell of embodiment 50, wherein expression of the protein is reduced as compared to a control cell lacking the at least one insertion or deletion.
52. The S. cerevisiae cell of embodiment 50 or 51, wherein expression of a messenger RNA encoding the protein is reduced as compared to a control cell lacking the at least one insertion or deletion.
53. The S. cerevisiae cell of embodiment 50, wherein expression of the protein is increased as compared to a control cell lacking the at least one insertion or deletion.
54. The S. cerevisiae cell of embodiment 50 or 53, wherein expression of a messenger RNA encoding the protein is increased as compared to a control cell lacking the at least one insertion or deletion.
55. The S. cerevisiae cell of any one of cells 50 to 54, wherein the at least one insertion or deletion is an insertion.
56. The S. cerevisiae cell of embodiment 55, wherein the insertion comprises the insertion of at least one nucleotide.
57. The S. cerevisiae cell of any one of cells 50 to 54, wherein the at least one insertion or deletion is a deletion.
58. The S. cerevisiae cell of embodiment 57, wherein the deletion comprises the deletion of at least one nucleotide.
59. The S. cerevisiae cell of any one of embodiments 50 to 58, wherein the at least one insertion or deletion is positioned within a region of the nucleic acid molecule selected from the group consisting of a promoter region, a 5′ untranslated region (UTR), an exon, an intron, a terminator region, and a 3′ UTR.
60. The S. cerevisiae cell of any one of embodiments 50 to 59, wherein the S. cerevisiae cell exhibits enhanced CBHI activity as compared to a control cell lacking the at least one at least one insertion or deletion.
61. A Saccharomyces cerevisiae cell comprising a transgene encoding a cellobiohydrolase I (CBHI) enzyme, a first modification affecting the expression or activity of a first protein, and a second modification affecting the expression or activity of a second protein, wherein a wildtype version of the first protein and a wildtype version of the second protein each comprise an amino acid sequence selected from the group consisting of SEQ ID NOs: 1 to 73.
62. The S. cerevisiae cell of embodiment 61, wherein the cell further comprises a third modification affecting the expression or activity of a third protein, wherein a wildtype version of the third protein comprises an amino acid sequence selected from the group consisting of SEQ ID NOs: 1 to 73.
63. The S. cerevisiae cell of embodiment 62, wherein the cell further comprises a fourth modification affecting the expression or activity of a fourth protein, wherein a wildtype version of the fourth protein comprises an amino acid sequence selected from the group consisting of SEQ ID NOs: 1 to 73.
64. The S. cerevisiae cell of embodiment 61, wherein the expression or activity of the first protein, the second protein, or both, is reduced as compared to a control cell lacking the at least one modification.
65. The S. cerevisiae cell of embodiment 61, wherein the expression or activity of the first protein, the second protein, or both is increased as compared to a control cell lacking the at least one modification.
66. The S. cerevisiae cell of embodiment 61, wherein expression of a messenger RNA (mRNA) molecule encoding the first protein, an mRNA molecule encoding the second protein, or both, is reduced as compared to a control cell lacking the at least one modification.
67. The S. cerevisiae cell of embodiment 61, wherein expression of a messenger RNA (mRNA) molecule encoding the first protein, an mRNA molecule encoding the second protein, or both, is increased as compared to a control cell lacking the at least one modification.
68. The S. cerevisiae cell of any one of embodiments 61 to 67, wherein: (a) the first modification results in a null allele of a nucleic acid molecule encoding the first protein; (b) the second modification results in a null allele of a nucleic acid molecule encoding the second protein; or (c) both (a) and (b).
69. The S. cerevisiae cell of embodiment 68, wherein: (a) the null allele of a nucleic acid molecule encoding the first protein comprises a premature stop codon as compared to the wildtype version of the first protein; (b) the null allele of a nucleic acid molecule encoding the second protein comprises a premature stop codon as compared to the wildtype version of the second protein; or both (a) and (b).
70. The S. cerevisiae cell of any one of embodiments 61 to 67, wherein: (a) the first modification comprises at least one amino acid substitution in the first protein; (b) the second modification comprises at least one amino acid substitution in the second protein; or (c) both (a) and (b).
71. The S. cerevisiae cell of any one of embodiments 61 to 67, wherein: (a) the first modification comprises an edit to a promoter region of a nucleic acid molecule encoding the first protein; (b) the second modification comprises an edit to a promoter region of a nucleic acid molecule encoding the second protein; or (c) both (a) and (b).
72. The S. cerevisiae cell of embodiment 71, wherein a wildtype version of the promoter region of the nucleic acid molecule encoding the first protein comprises a nucleic acid sequence as set forth in SEQ ID NO: 319 or SEQ ID NO: 321, and wherein a wildtype version of the promoter region of the nucleic acid molecule encoding the second protein comprises a nucleic acid sequence as set forth in SEQ ID NO: 319 or SEQ ID NO: 321.
73. The S. cerevisiae cell of any one of embodiments 61 to 67, wherein: (a) the first modification comprises an edit to a terminator region of a nucleic acid molecule encoding the first protein; (b) the second modification comprises an edit to a terminator region of a nucleic acid molecule encoding the second protein; or (c) both (a) and (b).
74. The S. cerevisiae cell of embodiment 73, wherein a wildtype version of the terminator region of the nucleic acid molecule encoding the first protein comprises a nucleic acid sequence selected from the group consisting of SEQ ID NOs: 311 to 318, and wherein a wildtype version of the terminator region of the nucleic acid molecule encoding the second protein comprises a nucleic acid sequence selected from the group consisting of SEQ ID NOs: 311 to 318.
75. The S. cerevisiae cell of any one of embodiments 61 to 67, wherein: (a) the first modification comprises an insertion of at least one nucleotide in a nucleic acid molecule encoding the first protein; (b) the second modification comprises an insertion of at least one nucleotide in a nucleic acid molecule encoding the second protein; or (c) both (a) and (b).
76. The S. cerevisiae cell of any one of embodiments 61 to 67, wherein: (a) the first modification comprises a deletion of at least one nucleotide in a nucleic acid molecule encoding the first protein; (b) the second modification comprises a deletion of at least one nucleotide in a nucleic acid molecule encoding the second protein; or (c) both (a) and (b).
77. The S. cerevisiae cell of any one of embodiments 61 to 76, wherein the S. cerevisiae cell exhibits enhanced CBHI activity as compared to a control cell lacking the first modification and the second modification.
78. The S. cerevisiae cell of any one of embodiments 1 to 77, wherein the transgene comprises a promoter operably linked to a nucleic acid sequence encoding the CBHI enzyme.
79. The S. cerevisiae cell of embodiment 78, wherein the promoter comprises SEQ ID NO: 319.
80. The S. cerevisiae cell of any one of embodiments 1 to 78, wherein the transgene comprises a terminator operably linked to a nucleic acid sequence encoding the CBHI enzyme.
81. The S. cerevisiae cell of any one of embodiments 1 to 79, wherein the transgene is codon optimized for S. cerevisiae.
82. The S. cerevisiae cell of any one of embodiments 1 to 79, wherein the transgene encodes a polypeptide comprising SEQ ID NO: 27.
83. A Saccharomyces cerevisiae cell comprising an Enolase-2 promoter sequence comprising a sequence selected from the group consisting of SEQ ID NOs: 101 to 105, 110 to 128, 205 to 209, and 214 to 232 operably linked to a nucleic acid sequence encoding a cellobiohydrolase I (CBHI) enzyme, wherein the nucleic acid sequence encoding CBHI comprises a sequence selected from the group consisting of SEQ ID NOs: 106 to 109, 100, 204, and 210 to 213.
84. A Saccharomyces cerevisiae cell comprising an Enolase-2 promoter sequence comprising a sequence selected from the group consisting of SEQ ID NOs: 101 to 105, 110 to 128, 205 to 209, and 214 to 232.
85. A Saccharomyces cerevisiae cell comprising a nucleic acid sequence encoding a cellobiohydrolase I (CBHI) enzyme, wherein the nucleic acid sequence encoding CBHI comprises a sequence selected from the group consisting of SEQ ID NOs: 106 to 109, 110, 204, and 200 to 213.
86. A Saccharomyces cerevisiae cell comprising an Enolase-2 promoter sequence comprising a sequence selected from the group consisting of SEQ ID NOs: 101 to 105, 110 to 128, 205 to 209, and 214 to 232 operably linked to a nucleic acid sequence encoding a cellobiohydrolase I (CBHI) enzyme comprising an amino acid sequence set forth in SEQ ID NO: 27 with a substitution from glycine to valine at position 22.
87. The S. cerevisiae cell of any one of embodiments 83, 84, and 86, wherein protein expression of the CHBI enzyme is increased as compared to a control cell lacking the Enolase-2 promoter sequence comprising the sequence selected from the group consisting of SEQ ID NOs: 101 to 105, 110 to 128, 205 to 209, and 214 to 232.
88. The S. cerevisiae cell of any one of embodiments 83, 84, and 86, wherein expression of a messenger RNA encoding the CHBI enzyme is increased as compared to a control cell lacking the Enolase-2 promoter sequence comprising the sequence selected from the group consisting of SEQ ID NOs: 101 to 105, 110 to 128, 205 to 209, and 214 to 232.
89. The S. cerevisiae cell of any one of embodiments 83, 84, and 86, wherein activity of the CHBI enzyme is increased as compared to a control cell lacking the Enolase-2 promoter sequence comprising the sequence selected from the group consisting of SEQ ID NOs: 101 to 105, 110 to 128, 205 to 209, and 214 to 232.
90. The S. cerevisiae cell of embodiment 83 or 85, wherein protein expression of the CHBI enzyme is increased as compared to a control cell lacking the nucleic acid sequence encoding the CBHI enzyme comprising the sequence selected from the group consisting of SEQ ID NOs: 106 to 109, 100, 204, and 210 to 213.
91. The S. cerevisiae cell of 83 or 85, wherein expression of a messenger RNA encoding the CHBI enzyme is increased as compared to a control cell lacking the nucleic acid sequence encoding the CBHI enzyme comprising the sequence selected from the group consisting of SEQ ID NOs: 106 to 109, 100, 204, and 210 to 213.
92. The S. cerevisiae cell of embodiment 83 or 85, wherein activity of the CHBI enzyme is increased as compared to a control cell lacking the nucleic acid sequence encoding the CBHI enzyme comprising the sequence selected from the group consisting of SEQ ID NOs: 106 to 109, 100, 204, and 210 to 213.
93. The S. cerevisiae cell of embodiment 86, wherein protein expression of the CHBI enzyme is increased as compared to a control cell lacking the nucleic acid sequence encoding the CBHI enzyme comprising the amino acid sequence set forth in SEQ ID NO: 27 with the substitution from glycine to valine at position 22.
94. The S. cerevisiae cell of 86, wherein expression of a messenger RNA encoding the CHBI enzyme is increased as compared to a control cell lacking the nucleic acid sequence encoding the CBHI enzyme comprising the amino acid sequence set forth in SEQ ID NO: 27 with the substitution from glycine to valine at position 22.
95. The S. cerevisiae cell of embodiment 86, wherein activity of the CHBI enzyme is increased as compared to a control cell lacking the nucleic acid sequence encoding the CBHI enzyme comprising the amino acid sequence set forth in SEQ ID NO: 27 with the substitution from glycine to valine at position 22.
96. A Saccharomyces cerevisiae cell comprising an edit listed in Table 1.
97. A Saccharomyces cerevisiae cell comprising an edit combination listed in Table 2.
98. A Saccharomyces cerevisiae cell comprising any combination of at least two, at least three, at least four, at least five, at least six, at least seven, at least eight, at least nine, or at least ten edits listed in Table 1.

### Examples

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Base Strain Construction

A CBH1 expression cassette was generated with a strong promoter and terminator so as to highly express CBHI from a single copy within the S. cerevisiae genome (strain CEN.PK). The construct was generated using strategies known in the art, including integration into the Leu2 site that would repair the auxotrophic deficiency of production of the amino acid leucine and allow for selection on leucine-negative plates. Integration was driven by native homologous recombination by flanking the construct with 500 nucleotides of homologous sequence to ensure high integration efficiency. Internal to the flanking homology sites, the expression cassette was generated with a modified enolase-2 (ENO2) promoter, a strong Kozak ribosome binding site, a codon-optimized CBH1 with a native secretion signal sequence, and a DIT1 terminator that has been shown previously to increase protein expression in yeast. The ENO2 promoter was modified from the native sequence by the addition of PAM sites (“TTTN” or “AAAN”) in non-conserved regions of the promoter, which were identified by alignments of the ENO2 promoter region across multiple Saccharomyces sequences, identifying non-conserved residues, and modifying those sites to include the PAM site. Four sites were identified to be non-conserved that required only a single nucleotide change to create a PAM site, and thus these four sites were targeted for modification.

The complete insertion construct was ordered as a fully-synthesized and sequence-validated cloned gene, and the insert was amplified by PCR using primers that flank the entire insertion sequence. Saccharomyces cerevisiae was made chemically competent by standard methods and 1 μg of linear DNA was added to the competent cells during transformation. The transformed yeast were washed twice in PBS and plated on agar plates without leucine. Eight colonies were then re-streaked to single colonies on a second agar plate without leucine. Colonies were then tested for integration by PCR using primers that flanked the insertion site. Positive colonies were moved forward for further engineering.

### Example II: Editing Cassette Preparation

The rationally-design edits focused on increased diversity, including genome-wide knock-outs genome-wide synthetic terminator insertions and genome-wide deletions, in addition to targeting the eno2 promoter, the integrated CBHI gene and protease and glycosylation pathways.

5 nM oligonucleotides synthesized on a chip were amplified using Q5 polymerase in 50 μL volumes. The PCR conditions were 95° C. for 1 minute; 8 rounds of 95° C. for 30 seconds/60° C. for 30 seconds/72° C. for 2.5 minutes; with a final hold at 72° C. for 5 minutes. Following amplification, the PCR products were subjected to SPRI cleanup, where 30 μL SPRI mix was added to the 50 μL PCR reactions and incubated for 2 minutes. The tubes were subjected to a magnetic field for 2 minutes, the liquid was removed, and the beads were washed 2× with 80% ethanol, allowing 1 minute between washes. After the final wash, the beads were allowed to dry for 2 minutes, 50 μL 0.5× TE pH 8.0 was added to the tubes, and the beads were vortexed to mix. The slurry was incubated at room temperature for 2 minutes, then subjected to the magnetic field for 2 minutes. The eluate was removed and the DNA quantified.

Following quantification, a second amplification procedure was carried out using a dilution of the eluate from the SPRI cleanup. PCR was performed under the following conditions: 95° C. for 1 minute; 18 rounds of 95° C. for 30 seconds/72° C. for 2.5 minutes; with a final hold at 72° C. for 5 minutes. Amplicons were checked on a 2% agarose gel and pools with the cleanest output(s) were identified. Amplification products appearing to have heterodimers or chimeras were not used.

### Example III: Backbone Preparation

Purified backbone vector was linearized by restriction enzyme digest with StuI. Up to 20 μg of purified backbone vector was in a 100 μL total volume in StuI-supplied buffer. Digestion was carried out at 30° C. for 16 hrs. Linear backbone was dialyzed to remove salt on 0.025 m MCE membrane for ˜60 min on nuclease-free water. Linear backbone concentration was measured using dye/fluorometer-based quantification.

### Example IV: Preparation of Competent Cells

The afternoon before transformation was to occur, 10 mL of YPAD was added to S. cerevisiae cells, and the culture was shaken at 250 rpm at 30° C. overnight. The next day, approximately 2 mL of the overnight culture was added to 100 mL of fresh YPAD in a 250-mL baffled flask and grown until the OD600 reading reached 0.3+/−0.05. The culture was then placed in a 30° C. incubator shaking at 250 rpm and allowed to grow for 4-5 hours, with the OD checked every hour. When the culture reached ˜1.5 OD600, two 50 mL aliquots of the culture were poured into two 50-mL conical vials and centrifuged at 4300 rpm for 2 minutes at room temperature. The supernatant was removed from the 50 mL conical tubes, avoiding disturbing the cell pellet. 25 mL of lithium acetate/DTT solution was added to each conical tube and the pellet was gently resuspended using an inoculating loop, needle, or long toothpick.

Following resuspension, both cell suspensions were transferred to a 250-mL flask and placed in the shaker to shake at 30° C. and 200 rpm for 30 minutes. After incubation was complete, the suspension was transferred to one 50-mL conical tube and centrifuged at 4300 RPM for 3 minutes. The supernatant was then discarded. From this point on, cold liquids were used and kept on ice until electroporation was complete. 50 mL of 1 M sorbitol was added to the cells and the pellet was resuspended. The cells were centrifuged at 4300 rpm for 3 minutes at 4° C., and the supernatant was discarded. The centrifugation and resuspension steps were repeated for a total of three washes. 50 μL of 1 M sorbitol was then added to one pellet, the cells were resuspended, then this aliquot of cells was transferred to the other tube and the second pellet was resuspended. The approximate volume of the cell suspension was measured, then brought to a 1 mL volume with cold 1 M sorbitol. The cell/sorbitol mixture and transferred into a 2-mm cuvette. Impedance measurement of the cells was measured in the cuvette.

Transformation was then performed using 500 ng of linear backbone along with 50 ng editing cassettes with the competent S. cerevisiae cells. 2 mm electroporation cuvettes were placed on ice and the plasmid/cassette mix was added to each corresponding cuvette. 100 μL of electrocompetent cells were added to each cuvette and the linear backbone and cassettes. Each sample was electroporated using the following conditions on a NEPAGENE electroporator: Poring pulse: 1800V, 5.0 second pulse length, 50.0 msec pulse interval, 1 pulse; Transfer pulse: 100 V, 50.0 msec pulse length, 50.0 msec pulse interval, with 3 pulses. Once the transformation process is complete, 900 μL of room temperature YPAD Sorbitol media was added to each cuvette. The cells were then transferred and suspended in a 15 mL tube and incubated shaking at 250 RPM at 30° C. for 3 hours. 9 mL of YPAD and 10 μL of Hygromycin B 1000× stock was added to the 15 mL tube.

### Example V: Screening of Edited Libraries for CBHI Expression

Library stocks were diluted and plated onto 245×245 mm YPD agar plates containing 250 μg/mL hygromycin (Teknova) using sterile glass beads. Libraries were diluted an appropriate amount to yield ˜1500-2000 colonies on the plates. Plates were incubated ˜48h at 30° C. and then stored at 4° C. until use. Colonies were picked using a QPix™ 420 (Molecular Devices) and deposited into sterile 1.2 mL square 96-well plates (Thomas Scientific) containing 300 μL YPD (250 μg/mL hygromycin (Gibco)). Plates were sealed (AirPore sheets (Qiagen)) and incubated for ˜36h in a shaker incubator (Climo-Shaker ISF1-X (Kuhner), 30° C., 85% humidity, 250 rpm). Plate cultures were then diluted 20-fold (15 μL culture into 285 μL medium) into new 96-well plates containing fresh YPD (250 μg/mL hygromycin). Production plates were incubated for 24h in a shaker incubator (Climo-Shaker ISF1-X (Kuhner), 30° C., 85% humidity, 250 rpm).

Production plates were centrifuged (Centrifuge 5920R, Eppendorf) at 3,000 g for 10 min to pellet cells. The supernatants from production plates were diluted 10-fold into CBH1 substrate solution (20 μL of supernatant with 180 μL of 50 mM sodium acetate (Sigma) pH 5.0, 100 mM sodium chloride (Sigma), 1 mM 4-nitrophenyl-beta-D-lactopyranoside (Carbosynth)) in clear flat bottom 96-well plates (Greiner Bio-One). Samples were thoroughly mixed and plates were heat sealed and incubated at 42° C. for 2h. Enzymatic reactions were quenched by the addition of 50 μL of 1M sodium carbonate (Sigma). CBH1 activity was determined by measuring the absorbance at 405 nm using a SpectraMax iD3 plate reader (Molecular Devices).

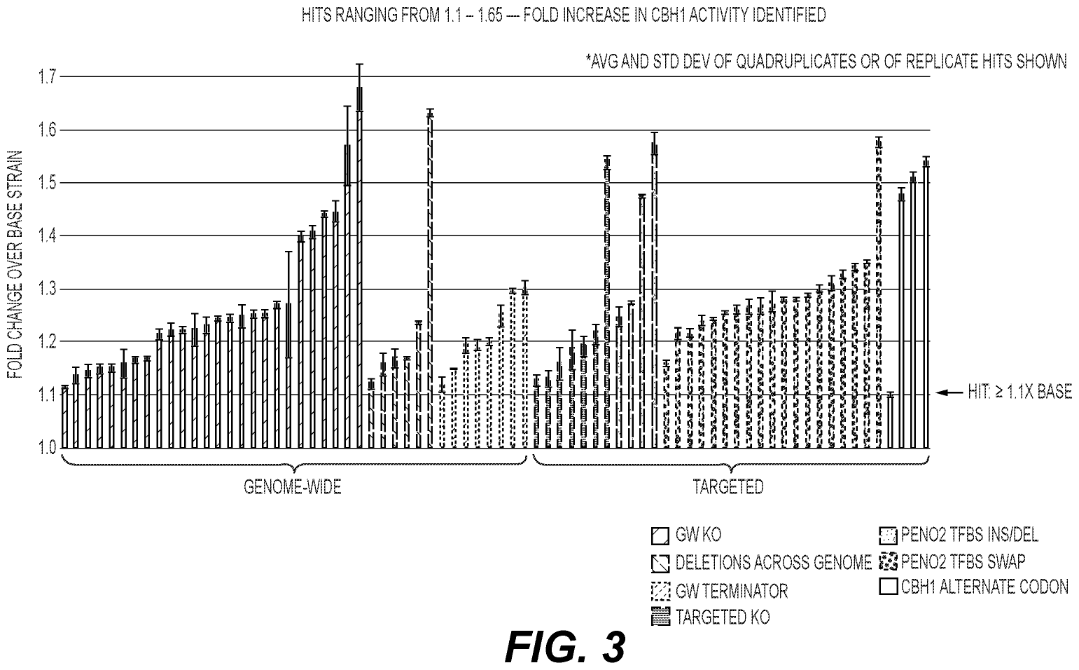

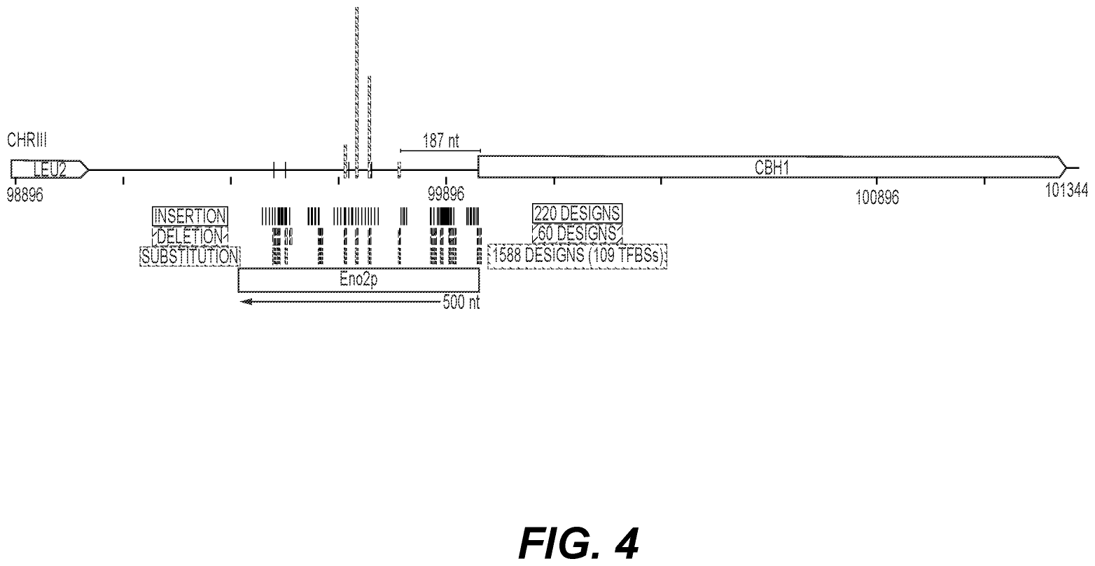

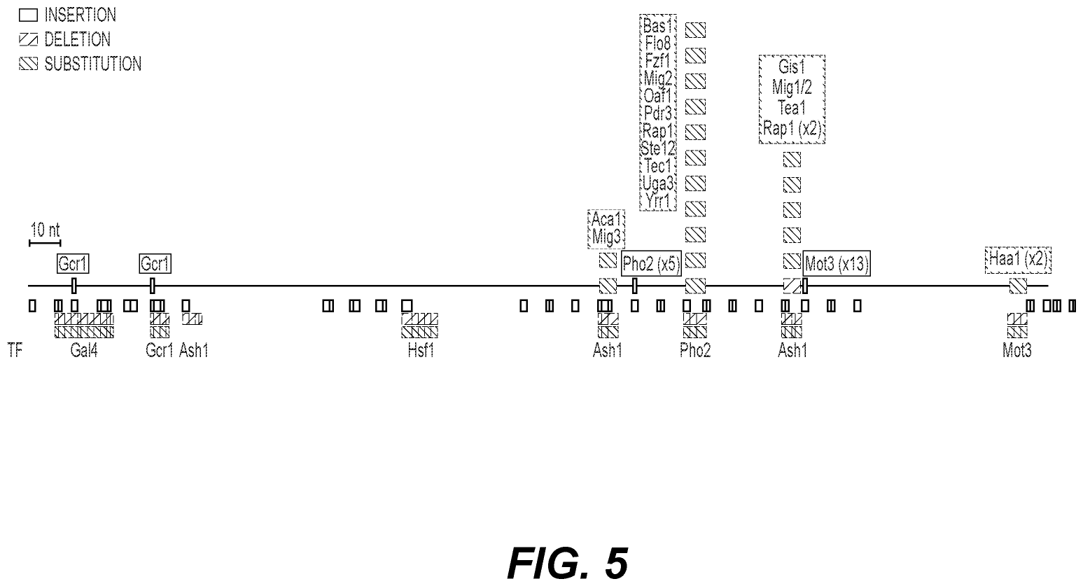

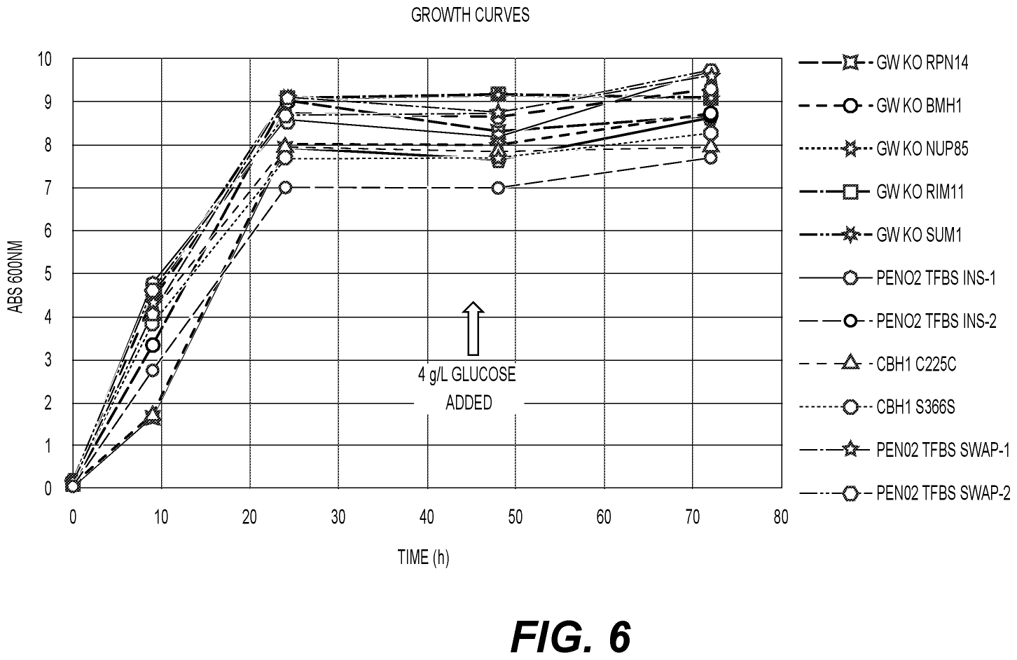

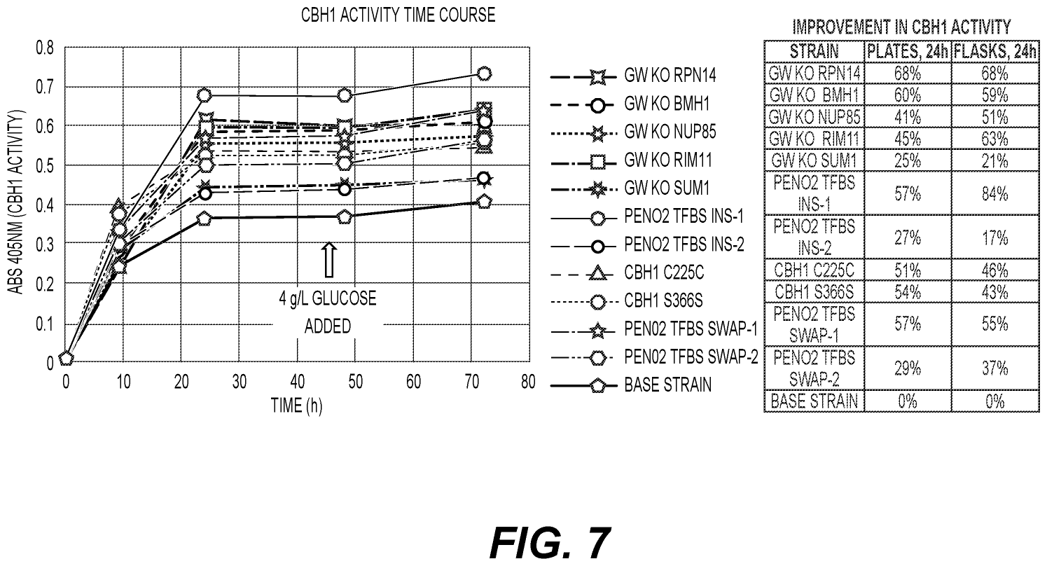

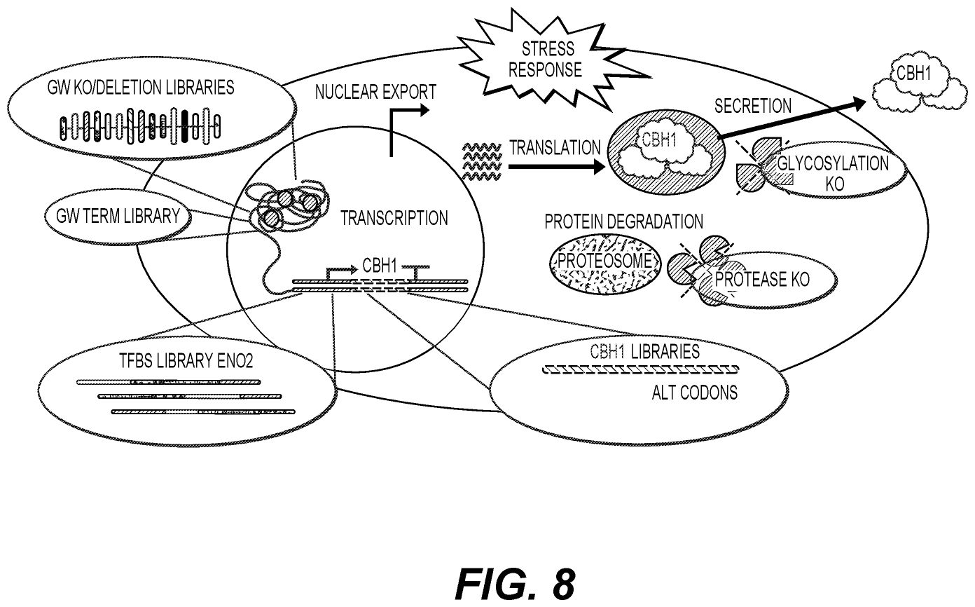

Each 96-well plate of samples contained 4 replicates of the base CBH1 expression strain to calculate the relative CBH1 activity of samples compared to the base strain control. Hits from the primary screen were re-tested in quadruplicate using a similar protocol as described above. FIG. 3 is a graph identifying the fold change over base strain for the different types of edits made to increase CBHI production. The amino acid or nucleic acid sequences for the genes and the edits (variants) made are listed in Table 1.

TABLE 1
Edits

| | | | | | | | | | In- | |
|---|---|---|---|---|---|---|---|---|---|---|
| | | | | | | | | | tended | Wild- |
| | | | | | | Com- | Com- | | Modi- | type |
| | | | | | | plete | plete | | fied | Pro- |
| | | | | | | Modi- | Allele | In- | Allele | tein |
| | | | | Edit | | fied | Se- | tended | Se- | Se- |
| | | | | De- | Allele | Allele | quence | Allele | quence | quence |
| Edit | Pheno- | Gene | Edit | scrip- | Annota- | Se- | SEQ | Se- | SEQ | SEQ |
| No. | type | Name | Type | tion | tion | quence | ID NO | quence | ID NO | ID NO |
| Edit | 1.67 | RPN1 | knock- | K15*** | Triple | ATCC | 74 | ATCC | 178 | 1 |
| 1 | 946 | 4 | out | | stop | AGTA | | AGTA | | |
| | 4 | | | | codon | TGAT | | TGAT | | |
| | | | | | inserted | TTTt | | TTTt | | |
| | | | | | at the | aata | | aata | | |
| | | | | | K15 | ataa | | ataa | | |
| | | | | | residue | CTGG | | CTGG | | |
| | | | | | position | AAGA | | AAGA | | |
| | | | | | of SEQ | AAAT | | AAAT | | |
| | | | | | ID NO: | GAT | | GAT | | |
| | | | | | 1 | | | | | |
| Edit | 1.59 | BMH | knock- | A15*** | Triple | TACC | 75 | TACC | 179 | 2 |
| 2 | 516 | 1 | out | | stop | TAGC | | TAGC | | |
| | 6 | | | | codon | CAAG | | CAAG | | |
| | | | | | inserted | TTGt | | TTGt | | |
| | | | | | at the | aata | | aata | | |
| | | | | | A15 | ataa | | ataa | | |
| | | | | | residue | gcgG | | GCCG | | |
| | | | | | position | AACG | | AACG | | |
| | | | | | of SEQ | TTAT | | TTAT | | |
| | | | | | ID NO: | GAAG | | GAAG | | |
| | | | | | 2 | AA | | AA | | |
| Edit | 1.40 | NUP | knock- | Q15*** | Triple | CTTA | 76 | CTTA | 180 | 3 |
| 3 | 727 | 85 | out | | stop | TGGA | | TGGA | | |
| | 2 | | | | codon | CGTC | | CGTC | | |
| | | | | | inserted | GATt | | GATt | | |
| | | | | | at the | aata | | aata | | |
| | | | | | Q15 | ataa | | ataa | | |
| | | | | | residue | TTTT | | TTTT | | |
| | | | | | position | TGGA | | TGGA | | |
| | | | | | of SEQ | CGAC | | CGAC | | |
| | | | | | ID NO: | GGA | | GGA | | |
| | | | | | 3 | | | | | |
| Edit | 1.22 | YNL | knock- | L15*** | Triple | ACTG | 77 | ACTG | 181 | 4 |
| 4 | 242 | 011C | out | | stop | CCAC | | CCAC | | |
| | 5 | | | | codon | AAAT | | AAAT | | |
| | | | | | inserted | TCAt | | TCAt | | |
| | | | | | at the | aata | | aata | | |
| | | | | | L15 | ataa | | ataa | | |
| | | | | | residue | tgtt | | TGCT | | |
| | | | | | position | tcag | | TTTC | | |
| | | | | | of SEQ | cAAT | | TAAT | | |
| | | | | | ID NO: | ATTT | | ATTT | | |
| | | | | | 4 | CTAT | | CTAT | | |
| | | | | | | CTTA | | CTTA | | |
| Edit | 1.26 | OCA | knock- | I15*** | Triple | GGGA | 78 | GGGA | 182 | 5 |
| 5 | 711 | 4 | out | | stop | TTGC | | TTGC | | |
| | 4 | | | | codon | TGAA | | TGAA | | |
| | | | | | inserted | GAAg | | GAAG | | |
| | | | | | at the | gcta | | GAta | | |
| | | | | | I15 | ataa | | ataa | | |
| | | | | | residue | taaT | | taaT | | |
| | | | | | position | GTTC | | GTTC | | |
| | | | | | of SEQ | TAAA | | TAAA | | |
| | | | | | ID NO: | GTGG | | GTGG | | |
| | | | | | 5 | AG | | AG | | |
| Edit | 1.24 | BMH | knock- | A15*** | Triple | TACC | 79 | TACC | 183 | 6 |
| 6 | 883 | 2 | out | | stop | TAGC | | TAGC | | |
| | 2 | | | | codon | TAAA | | TAAA | | |
| | | | | | inserted | TTAt | | TTAt | | |
| | | | | | at the | aata | | aata | | |
| | | | | | A15 | ataa | | ataa | | |
| | | | | | residue | GCCG | | GCCG | | |
| | | | | | position | AACG | | AACG | | |
| | | | | | of SEQ | TTAT | | TTAT | | |
| | | | | | ID NO: | GAA | | GAA | | |
| | | | | | 6 | | | | | |
| Edit | 1.13 | STM | knock- | A15*** | Triple | AACG | 80 | AACG | 184 | 7 |
| 7 | 101 | 1 | out | | stop | ACGT | | ACGT | | |
| | 8 | | | | codon | CGAA | | CGAA | | |
| | | | | | inserted | GACt | | GACt | | |
| | | | | | at the | aata | | aata | | |
| | | | | | A15 | ataa | | ataa | | |
| | | | | | residue | GTCG | | GTCG | | |
| | | | | | position | TTTT | | TTTT | | |
| | | | | | of SEQ | GCCA | | GCCA | | |
| | | | | | ID NO: | CCA | | CCA | | |
| | | | | | 7 | | | | | |
| Edit | 1.16 | SLM | knock- | N15*** | Triple | AAAG | 81 | AAAG | 185 | 8 |
| 8 | 859 | 4 | out | | stop | GGTT | | GGTT | | |
| | 6 | | | | codon | TCTG | | TCTG | | |
| | | | | | inserted | GAAt | | GAAt | | |
| | | | | | at the | aata | | aata | | |
| | | | | | N15 | ataa | | ataa | | |
| | | | | | residue | AAAC | | AAAC | | |
| | | | | | position | CGTA | | CGTA | | |
| | | | | | of SEQ | TGAT | | TGAT | | |
| | | | | | ID NO: | TTG | | TTG | | |
| | | | | | 8 | | | | | |
| Edit | 1.22 | APJ1 | knock- | A15*** | Triple | TCTT | 82 | TCTT | 186 | 9 |
| 9 | 344 | | out | | stop | TGAA | | TGAA | | |
| | 1 | | | | codon | CGTT | | CGTT | | |
| | | | | | inserted | ACTt | | ACTt | | |
| | | | | | at the | aata | | aata | | |
| | | | | | A15 | ataa | | ataa | | |
| | | | | | residue | TCCA | | TCCA | | |
| | | | | | position | CATC | | CATC | | |
| | | | | | of SEQ | TGAG | | TGAG | | |
| | | | | | ID NO: | ATT | | ATT | | |
| | | | | | 9 | | | | | |
| Edit | 1.11 | AGE | knock- | L15*** | Triple | GCAT | 83 | GCAT | 187 | 10 |
| 10 | 476 | 2 | out | | stop | TAAG | | TAAG | | |
| | 7 | | | | codon | TGCT | | TGCT | | |
| | | | | | inserted | CTTt | | CTTt | | |
| | | | | | at the | aata | | aata | | |
| | | | | | L15 | ataa | | ataa | | |
| | | | | | residue | CCAG | | CCAG | | |
| | | | | | position | GAAA | | GAAA | | |
| | | | | | of SEQ | CAGT | | CAGT | | |
| | | | | | ID NO: | CAT | | CAT | | |
| | | | | | 10 | | | | | |
| Edit | 1.27 | STB5 | knock- | R15*** | Triple | GCAC | 84 | GCAC | 188 | 11 |
| 11 | 117 | | out | | stop | ATCA | | ATCA | | |
| | 6 | | | | codon | AGGC | | AGGC | | |
| | | | | | inserted | GGGc | | GGGA | | |
| | | | | | at the | gtag | | GATC | | |
| | | | | | R15 | ccag | | ACAA | | |
| | | | | | residue | taat | | taat | | |
| | | | | | position | aata | | aata | | |
| | | | | | of SEQ | aGAA | | aGAA | | |
| | | | | | ID NO: | TTGT | | TTGT | | |
| | | | | | 11 | ATTC | | ATTC | | |
| | | | | | | GTGC | | GTGC | | |
| Edit | 1.14 | RPL8 | knock- | K15*** | Triple | GCTC | 85 | GCTC | 189 | 12 |
| 12 | 929 | A | out | | stop | CATT | | CATT | | |
| | 9 | | | | codon | CGGT | | CGGT | | |
| | | | | | inserted | GCTt | | GCTt | | |
| | | | | | at the | aata | | aata | | |
| | | | | | K15 | ataa | | ataa | | |
| | | | | | residue | AAGT | | AAGT | | |
| | | | | | position | CTAA | | CTAA | | |
| | | | | | of SEQ | CAAG | | CAAG | | |
| | | | | | ID NO: | ACT | | ACT | | |
| | | | | | 12 | | | | | |
| Edit | 1.44 | YHR | knock- | S15*** | Triple | AAGC | 86 | AAGC | 190 | 13 |
| 13 | 053 | 033W | out | | stop | TGGG | | TGGG | | |
| | 2 | | | | codon | CAGT | | CAGT | | |
| | | | | | inserted | TCGt | | TCGt | | |
| | | | | | at the | aata | | aata | | |
| | | | | | S15 | ataa | | ataa | | |
| | | | | | residue | GACG | | GACG | | |
| | | | | | position | AGAG | | AGAG | | |
| | | | | | of SEQ | CACA | | CACA | | |
| | | | | | ID NO: | AAA | | AAA | | |
| | | | | | 13 | | | | | |
| Edit | 1.44 | RIM1 | knock- | I15*** | Triple | AATC | 87 | AATC | 191 | 14 |
| 14 | 669 | 1 | out | | stop | TCAG | | TCAG | | |
| | 8 | | | | codon | TAAT | | TAAT | | |
| | | | | | inserted | AACt | | AACt | | |
| | | | | | at the | aata | | aata | | |
| | | | | | I15 | ataa | | ataa | | |
| | | | | | residue | AAAC | | AAAC | | |
| | | | | | position | AGGT | | AGGT | | |
| | | | | | of SEQ | TTAC | | TTAC | | |
| | | | | | ID NO: | TAC | | TAC | | |
| | | | | | 14 | | | | | |
| Edit | 1.39 | NNK | knock- | M15*** | Triple | ACGT | 88 | ACGT | 192 | 15 |
| 15 | 736 | 1 | out | | stop | CGCA | | CGCA | | |
| | 9 | | | | codon | GCGA | | GCGA | | |
| | | | | | inserted | CAGc | | CAGC | | |
| | | | | | at the | tgag | | TTCG | | |
| | | | | | M15 | acag | | ACAA | | |
| | | | | | residue | AATG | | AATG | | |
| | | | | | position | GGAG | | GGAG | | |
| | | | | | of SEQ | TCCG | | TCCG | | |
| | | | | | ID NO: | taat | | taat | | |
| | | | | | 15 | aata | | aata | | |
| | | | | | | aTCA | | aTCA | | |
| | | | | | | CGTT | | CGTT | | |
| | | | | | | CATC | | CATC | | |
| | | | | | | ACAA | | ACAA | | |
| Edit | 1.25 | SUM | knock- | E15*** | Triple | GATA | 89 | GATA | 193 | 16 |
| 16 | 349 | 1 | out | | stop | ACAT | | ACAT | | |
| | 4 | | | | codon | AACC | | AACC | | |
| | | | | | inserted | AATt | | AATt | | |
| | | | | | at the | aata | | aata | | |
| | | | | | E15 | ataa | | ataa | | |
| | | | | | residue | CTTC | | CTTC | | |
| | | | | | position | CGTC | | CGTC | | |
| | | | | | of SEQ | TGGA | | TGGA | | |
| | | | | | ID NO: | CCC | | CCC | | |
| | | | | | 16 | | | | | |
| Edit | 1.25 | YKL | knock- | S15*** | Triple | TTTA | 90 | TTTA | 194 | 17 |
| 17 | 280 | 071W | out | | stop | TCAT | | TCAT | | |
| | 9 | | | | codon | CGGT | | CGGT | | |
| | | | | | inserted | GGTt | | GGTt | | |
| | | | | | at the | aata | | aata | | |
| | | | | | S15 | ataa | | ataa | | |
| | | | | | residue | ATTG | | ATTG | | |
| | | | | | position | GTTT | | GTTT | | |
| | | | | | of SEQ | TAAT | | TAAT | | |
| | | | | | ID NO: | TTG | | TTG | | |
| | | | | | 17 | | | | | |
| Edit | 1.24 | YOR | knock- | C15*** | Triple | ACTG | 91 | ACTG | 195 | 18 |
| 18 | 527 | 161C- | out | | stop | GAAA | | GAAA | | |
| | 3 | C | | | codon | AATG | | AATG | | |
| | | | | | inserted | GCTt | | GCTt | | |
| | | | | | at the | aata | | aata | | |
| | | | | | C15 | ataa | | ataa | | |
| | | | | | residue | AGGC | | AGGC | | |
| | | | | | position | AGAT | | AGAT | | |
| | | | | | of SEQ | Tctg | | TTTG | | |
| | | | | | ID NO: | gcgt | | GCAA | | |
| | | | | | 18 | ctCC | | GCCC | | |
| | | | | | | ACTG | | ACTG | | |
| | | | | | | TTTT | | TTTT | | |
| | | | | | | TCGC | | TCGC | | |
| | | | | | | A | | A | | |
| Edit | 1.24 | POC4 | knock- | G15*** | Triple | ATTG | 92 | ATTG | 196 | 19 |
| 19 | 732 | | out | | stop | AATC | | AATC | | |
| | 8 | | | | codon | AGAA | | AGAA | | |
| | | | | | inserted | TCTt | | TCTt | | |
| | | | | | at the | aata | | aata | | |
| | | | | | G15 | ataa | | ataa | | |
| | | | | | residue | CAGC | | CAGC | | |
| | | | | | position | CGAC | | CGAC | | |
| | | | | | of SEQ | GCTG | | GCTG | | |
| | | | | | ID NO: | GAT | | GAT | | |
| | | | | | 19 | | | | | |
| Edit | 1.21 | GLO | knock- | T15*** | Triple | GCCA | 93 | GCCA | 197 | 20 |
| 20 | 444 | 3 | out | | stop | CGGA | | CGGA | | |
| | 2 | | | | codon | GCAG | | GCAG | | |
| | | | | | inserted | ACTt | | ACTt | | |
| | | | | | at the | aata | | aata | | |
| | | | | | T15 | ataa | | ataa | | |
| | | | | | residue | GTTT | | GTTT | | |
| | | | | | position | TTCA | | TTCA | | |
| | | | | | of SEQ | GAAG | | GAAG | | |
| | | | | | ID NO: | CTA | | CTA | | |
| | | | | | 20 | | | | | |
| Edit | 1.13 | TGS1 | knock- | A15 ** | Triple | AAAA | 94 | AAAA | 198 | 21 |
| 21 | 565 | | out | | stop | TAAA | | TAAA | | |
| | 4 | | | | codon | ACAT | | ACAT | | |
| | | | | | inserted | GCGt | | GCGt | | |
| | | | | | at the | aata | | aata | | |
| | | | | | A15 | ataa | | ataa | | |
| | | | | | residue | AGAA | | AGAA | | |
| | | | | | position | AACA | | AACA | | |
| | | | | | of SEQ | TCAT | | TCAT | | |
| | | | | | ID NO: | TCC | | TCC | | |
| | | | | | 21 | | | | | |
| Edit | 1.22 | RIM9 | knock- | T15*** | Triple | TTTT | 95 | TTTT | 199 | 22 |
| 22 | 197 | | out | | stop | TGCT | | TGCT | | |
| | 9 | | | | codon | AGCA | | AGCA | | |
| | | | | | inserted | ATCt | | ATCt | | |
| | | | | | at the | aata | | aata | | |
| | | | | | T15 | ataa | | ataa | | |
| | | | | | residue | TTCG | | TTCG | | |
| | | | | | position | AAAT | | AAAT | | |
| | | | | | of SEQ | ACTT | | ACTT | | |
| | | | | | ID NO: | CCG | | CCG | | |
| | | | | | 22 | | | | | |
| Edit | 1.26 | UBX | knock- | V15*** | Triple | CTCT | 96 | CTCT | 200 | 23 |
| 23 | 925 | 6 | out | | stop | TTCA | | TTCA | | |
| | 2 | | | | codon | TGAT | | TGAT | | |
| | | | | | inserted | CGAt | | CGAt | | |
| | | | | | at the | aata | | aata | | |
| | | | | | V15 | ataa | | ataa | | |
| | | | | | residue | GATT | | GATT | | |
| | | | | | position | ACTC | | ACTC | | |
| | | | | | of SEQ | TCAT | | TCAT | | |
| | | | | | ID NO: | ACT | | ACT | | |
| | | | | | 23 | | | | | |
| Edit | 1.14 | YKL | knock- | S15*** | Triple | GCGG | 97 | GCGG | 201 | 24 |
| 24 | 456 | 075C | out | | stop | CCAA | | CCAA | | |
| | | | | | codon | CGAG | | CGAG | | |
| | | | | | inserted | CCAt | | CCAt | | |
| | | | | | at the | aata | | aata | | |
| | | | | | S15 | ataa | | ataa | | |
| | | | | | residue | GACT | | GACT | | |
| | | | | | position | GTAC | | GTAC | | |
| | | | | | of SEQ | CTGT | | CTGT | | |
| | | | | | ID NO: | AAA | | AAA | | |
| | | | | | 24 | | | | | |
| Edit | 1.15 | OCA | knock- | N15*** | Triple | TCGA | 98 | TCGA | 202 | 25 |
| 25 | 141 | 6 | out | | stop | CCGT | | CCGT | | |
| | 1 | | | | codon | ACAG | | ACAG | | |
| | | | | | inserted | CCAt | | CCAt | | |
| | | | | | at the | aata | | aata | | |
| | | | | | N15 | ataa | | ataa | | |
| | | | | | residue | AGAG | | AGAG | | |
| | | | | | position | GATC | | GATC | | |
| | | | | | of SEQ | TTAC | | TTAC | | |
| | | | | | ID NO: | CCA | | CCA | | |
| | | | | | 25 | | | | | |
| Edit | 1.16 | MKT | knock- | L15*** | Triple | CTTT | 99 | CTTT | 203 | 26 |
| 26 | 579 | 1 | out | | stop | TCGA | | TCGA | | |
| | 9 | | | | codon | AAGA | | AAGA | | |
| | | | | | inserted | GGTt | | GGTt | | |
| | | | | | at the | aata | | aata | | |
| | | | | | L15 | ataa | | ataa | | |
| | | | | | residue | TCCT | | TCCT | | |
| | | | | | position | ATGC | | ATGC | | |
| | | | | | of SEQ | CATT | | CATT | | |
| | | | | | ID NO: | GAG | | GAG | | |
| | | | | | 26 | | | | | |
| Edit | 1.57 | CBH | sub- | G22V | Amino | AAAG | 100 | AAAG | 204 | 27 |
| 27 | 087 | 1 | stitu- | | acid | CACA | | CACA | | |
| | 7 | | tion | | substi- | GCAA | | GCAA | | |
| | | | | | tution | GCCg | | GCCg | | |
| | | | | | from G | tgAC | | tgAC | | |
| | | | | | to V at | TGCA | | TGCA | | |
| | | | | | residue | ACAG | | ACAG | | |
| | | | | | position | CAGA | | CAGA | | |
| | | | | | 22 of | A | | A | | |
| | | | | | SEQ ID | | | | | |
| | | | | | NO: 27 | | | | | |
| Edit | 1.57 | CBH | eno2 | chrIII_1 | Insertion | TTGG | 101 | TTGG | 205 | 27 |
| 28 | 278 | 1 | promo- | 87TTA | of | TTGT | | TTGT | | |
| | 6 | | ter | ATT | “TTAAT | ATTG | | ATTG | | |
| | | | trans- | | T” into | ATCg | | ATCA | | |
| | | | crip- | | SEQ ID | gTTt | | TTTt | | |
| | | | tion | | NO: 319 | taat | | taat | | |
| | | | factor | | | tGGT | | tGGT | | |
| | | | bind- | | | TCAT | | TCAT | | |
| | | | ing | | | CGTG | | CGTG | | |
| | | | site | | | GTTC | | GTTC | | |
| | | | indel | | | | | | | |
| Edit | 1.47 | CBH | eno2 | chrIII_ | Deletion | CATT | 102 | CATT | 206 | 27 |
| 29 | 777 | 1 | promo- | TTGAT | of | GCTT | | GCTT | | |
| | | | ter | 187----- | “TTGAT” | TCTG | | TCTG | | |
| | | | trans- | | from | GCTC | | GCTT | | |
| | | | crip- | | SEQ ID | TTAC | | TGAT | | |
| | | | tion | | NO: 319 | TATC | | CTTA | | |
| | | | factor | | | ATTT | | CTAT | | |
| | | | bind- | | | GGA | | CAT | | |
| | | | ing | | | | | | | |
| | | | site | | | | | | | |
| | | | indel | | | | | | | |
| Edit | 1.25 | CBH | eno2 | chrIII_1 | Insertion | CTCC | 103 | CTCC | 207 | 27 |
| 30 | 905 | 1 | promo- | 87AAG | of | ATTG | | ATTG | | |
| | 3 | | ter | GTT | “AAGG | CTTT | | CTTT | | |
| | | | trans- | | TT” into | CTGa | | CTGG | | |
| | | | crip- | | SEQ ID | aggg | | CTTT | | |
| | | | tion | | NO: 319 | aATC | | GATC | | |
| | | | factor | | | aagg | | aagg | | |
| | | | bind- | | | ttTT | | ttTT | | |
| | | | ing | | | ACTA | | ACTA | | |
| | | | site | | | TCAT | | TCAT | | |
| | | | indel | | | TTGG | | TTGG | | |
| | | | | | | A | | A | | |
| Edit | 1.25 | CBH | eno2 | chrIII_1 | Insertion | TCTC | 104 | TCTC | 208 | 27 |
| 31 | 367 | 1 | promo- | 87AAG | of | CATT | | CATT | | |
| | 5 | | ter | GTT | “AAGG | GCTT | | GCTT | | |
| | | | trans- | | TT” into | TCTa | | TCTG | | |
| | | | crip- | | SEQ ID | aagg | | GCTT | | |
| | | | tion | | NO: 319 | gGAT | | TGAT | | |
| | | | factor | | | Caag | | Caag | | |
| | | | bind- | | | gttT | | gttT | | |
| | | | ing | | | TACT | | TACT | | |
| | | | site | | | ATCA | | ATCA | | |
| | | | indel | | | TTTG | | TTTG | | |
| | | | | | | GA | | GA | | |
| Edit | 1.27 | CBH | eno2 | chrIII_1 | Insertion | CACC | 105 | CACC | 209 | 27 |
| 32 | 249 | 1 | promo- | 87CAT | of | AACT | | AACT | | |
| | 9 | | ter | CC | “CATCC” | TGCG | | TGCG | | |
| | | | trans- | | into | GAAC | | GAAC | | |
| | | | crip- | | SEQ ID | atcc | | atcc | | |
| | | | tion | | NO: 319 | AGTG | | AGTG | | |
| | | | factor | | | GAAT | | GAAT | | |
| | | | bind- | | | CCCG | | CCCG | | |
| | | | ing | | | TTC | | TTC | | |
| | | | site | | | | | | | |
| | | | indel | | | | | | | |
| Edit | 1.51 | CBH | alter- | C225C | Codon | ATAG | 106 | ATAG | 210 | 27 |
| 33 | 136 | 1 | nate | | replace- | GTGA | | GTGA | | |
| | 4 | | codon | | ment at | TCAT | | TCAT | | |
| | | | | | position | GGCa | | GGCT | | |
| | | | | | 225 of | gcTG | | CCTG | | |
| | | | | | SEQ ID | Ttgc | | Ttgc | | |
| | | | | | NO: 27 | GCTG | | GCTG | | |
| | | | | | | AAAT | | AAAT | | |
| | | | | | | GGAT | | GGAT | | |
| | | | | | | GTG | | GTG | | |
| Edit | 1.54 | CBH | alter- | S366S | Codon | GATA | 107 | GATA | 211 | 27 |
| 34 | 017 | 1 | nate | | replace- | CCGA | | CCGA | | |
| | 3 | | codon | | ment at | TGAC | | TGAC | | |
| | | | | | position | TTCt | | TTCt | | |
| | | | | | 366 of | caca | | caCA | | |
| | | | | | SEQ ID | gCAT | | ACAT | | |
| | | | | | NO: 27 | GGAG | | GGAG | | |
| | | | | | | GCCT | | GCCT | | |
| | | | | | | GGCA | | GGCA | | |
| Edit | 1.47 | CBH | alter- | G275G | Codon | ACTT | 108 | ACTT | 212 | 27 |
| 35 | 855 | 1 | nate | | replace- | GCGA | | GCGA | | |
| | 3 | | codon | | ment at | TCCA | | TCCA | | |
| | | | | | position | GACg | | GACg | | |
| | | | | | 275 of | ggTG | | ggTG | | |
| | | | | | SEQ ID | Tgac | | TGAT | | |
| | | | | | NO: 27 | ttca | | TTTA | | |
| | | | | | | acCC | | ATCC | | |
| | | | | | | ATAC | | ATAC | | |
| | | | | | | CGTA | | CGTA | | |
| | | | | | | TGGG | | TGGG | | |
| | | | | | | A | | A | | |
| Edit | 1.10 | CBH | alter- | Y65Y | Codon | CACG | 109 | CACG | 213 | 27 |
| 36 | 563 | 1 | nate | | replace- | ACGT | | ACGT | | |
| | 4 | | codon | | ment at | TAAT | | TAAT | | |
| | | | | | position | GGTt | | GGTt | | |
| | | | | | 65 of | acac | | acAC | | |
| | | | | | SEQ ID | cAAT | | AAAT | | |
| | | | | | NO: 27 | TGCT | | TGCT | | |
| | | | | | | ACAC | | ACAC | | |
| | | | | | | TGGA | | TGGA | | |
| Edit | 1.58 | CBH | eno2 | chrIII_ | Replace- | CATT | 110 | CATT | 214 | 27 |
| 37 | 639 | 1 | promo- | TTGAT | ment of | GCTT | | GCTT | | |
| | 9 | | ter | C(187, | “TTGAT | TCTG | | TCTG | | |
| | | | trans- | 192)AC | C” with | GCTa | | GCTa | | |
| | | | crip- | ACCC | “ACAC | cacc | | cacc | | |
| | | | tion | GTAC | CCGTA | cgta | | cgta | | |
| | | | factor | AC | CAC” | cacT | | cacT | | |
| | | | bind- | | (SEQ ID | TACT | | TACT | | |
| | | | ing | | NO: 282) | ATCA | | ATCA | | |
| | | | site | | | TTTG | | TTTG | | |
| | | | indel | | | GA | | GA | | |
| Edit | 1.34 | CBH | eno2 | chrIII_ | Replace- | CATT | 111 | CATT | 215 | 27 |
| 38 | 986 | 1 | promo- | TTGAT | ment of | GCTT | | GCTT | | |
| | 9 | | ter | C(187, | “TTGAT | TCTG | | TCTG | | |
| | | | trans- | 192)CG | C” with | GCTc | | GCTc | | |
| | | | crip- | GAGT | “CGGA | ggag | | ggag | | |
| | | | tion | AACC | GTAAC | taac | | taac | | |
| | | | factor | GCGC | CGCGC | cgcg | | cgcg | | |
| | | | bind- | CG | CG” | ccgT | | ccgT | | |
| | | | ing | | (SEQ ID | TACT | | TACT | | |
| | | | site | | NO: 283) | ATCA | | ATCA | | |
| | | | indel | | | TTTG | | TTTG | | |
| | | | | | | GA | | GA | | |
| Edit | 1.34 | CBH | eno2 | chrIII_ | Replace- | GTTC | 112 | GTTC | 216 | 27 |
| 39 | 165 | 1 | promo- | TTAAT | ment of | ATCG | | ATCG | | |
| | 6 | | ter | TT(187, | “TTAAT | TGGT | | TGGT | | |
| | | | trans- | 193)C | TT” with | TCAc | | TCAc | | |
| | | | crip- | GGGG | “CGGG | gggg | | gggg | | |
| | | | tion | TTTTC | GTTTT | tttt | | tttt | | |
| | | | factor | T | CT” | ctTT | | ctTT | | |
| | | | bind- | | (SEQ ID | TTTC | | TTTC | | |
| | | | ing | | NO: 284) | TCCA | | TCCA | | |
| | | | site | | | TTGC | | TTGC | | |
| | | | indel | | | T | | T | | |
| Edit | 1.30 | CBH | eno2 | chrIII_ | Replace- | GTTC | 113 | GTTC | 217 | 27 |
| 40 | 141 | 1 | promo- | TTAAT | ment of | ATCG | | ATCG | | |
| | 3 | | ter | TT(187, | “TTAAT | TGGT | | TGGT | | |
| | | | trans- | 193)T | TT” with | TCAt | | TCAt | | |
| | | | crip- | CCGC | “TCCGC | ccgc | | ccgc | | |
| | | | tion | GGG | GGG” | gggT | | gggT | | |
| | | | factor | | | TTTT | | TTTT | | |
| | | | bind- | | | CTCC | | CTCC | | |
| | | | ing | | | ATTG | | ATTG | | |
| | | | site | | | CT | | CT | | |
| | | | indel | | | | | | | |
| Edit | 1.32 | CBH | eno2 | chrIII_ | Replace- | GTTC | 114 | GTTC | 218 | 27 |
| 41 | 769 | 1 | promo- | TTAAT | ment of | ATCG | | ATCG | | |
| | 4 | | ter | TT(187, | “TTAAT | TGGT | | TGGT | | |
| | | | trans- | 193)C | TT” with | TCAc | | TCAc | | |
| | | | crip- | GCGG | “CGCG | gcgg | | gcgg | | |
| | | | tion | TTTTT | GTTTTT” | tttt | | tttt | | |
| | | | factor | | (SEQ | tTTT | | tTTT | | |
| | | | bind- | | ID NO: | TTCT | | TTCT | | |
| | | | ing | | 285) | CCAT | | CCAT | | |
| | | | site | | | TGCT | | TGCT | | |
| | | | indel | | | | | | | |
| Edit | 1.26 | CBH | eno2 | chrIII_ | Replace- | GTTT | 115 | GTTT | 219 | 27 |
| 42 | 691 | 1 | promo- | TTGAT | ment of | CTTT | | CTTT | | |
| | 9 | | ter | C(187, | “TTGAT | GGTT | | GGTT | | |
| | | | trans- | 192)TG | C” with | GTAt | | GTAt | | |
| | | | crip- | ACGT | “TGAC | gacg | | gacg | | |
| | | | tion | CA | GTCA” | tcaA | | tcaA | | |
| | | | factor | | | TTTG | | TTTG | | |
| | | | bind- | | | GTTC | | GTTC | | |
| | | | ing | | | ATCG | | ATCG | | |
| | | | site | | | TG | | TG | | |
| | | | indel | | | | | | | |
| Edit | 1.29 | CBH | eno2 | chrIII_ | Replace- | GTTC | 116 | GTTC | 220 | 27 |
| 43 | 566 | 1 | promo- | TTAAT | ment of | ATCG | | ATCG | | |
| | 4 | | ter | TT(187, | “TTAAT | TGGT | | TGGT | | |
| | | | trans- | 193)A | TT” with | TCAa | | TCAa | | |
| | | | crip- | CACC | “ACAC | cacc | | cacc | | |
| | | | tion | CGTA | CCGTA | cgta | | cgta | | |
| | | | factor | CAC | CAC” | cacT | | cacT | | |
| | | | bind- | | (SEQ ID | TTTT | | TTTT | | |
| | | | ing | | NO: 286) | CTCC | | CTCC | | |
| | | | site | | | ATTG | | ATTG | | |
| | | | indel | | | CT | | CT | | |
| Edit | 1.26 | CBH | eno2 | chrIII_ | Replace- | GTTT | 117 | GTTT | 221 | 27 |
| 44 | 034 | 1 | promo- | TTGAT | ment of | CTTT | | CTTT | | |
| | 8 | | ter | C(187, | “TTGAT | GGTT | | GGTT | | |
| | | | trans- | 192)ATT | C” with | GTAa | | GTAa | | |
| | | | crip- | TTGC | “ATTTT | tttt | | tttt | | |
| | | | tion | GGGG | GCGGG | gcgg | | gcgg | | |
| | | | factor | | G” (SEQ | ggAT | | ggAT | | |
| | | | bind- | | ID NO: | TTGG | | TTGG | | |
| | | | ing | | 287) | TTCA | | TTCA | | |
| | | | site | | | TCGT | | TCGT | | |
| | | | indel | | | G | | G | | |
| Edit | 1.26 | CBH | eno2 | chrIII_ | Replace- | GTTC | 118 | GTTC | 222 | 27 |
| 45 | 774 | 1 | promo- | TTAAT | ment of | ATCG | | ATCG | | |
| | | | ter | TT(187, | “TTAAT | TGGT | | TGGT | | |
| | | | trans- | 193)C | TT” with | TCAc | | TCAC | | |
| | | | crip- | GGAT | “CGGA | ggat | | ggat | | |
| | | | tion | CTAA | TCTAA” | ctaa | | ctaa | | |
| | | | factor | | | TTTT | | TTTT | | |
| | | | bind- | | | TCTC | | TCTC | | |
| | | | ing | | | CATT | | CATT | | |
| | | | site | | | GCT | | GCT | | |
| | | | indel | | | | | | | |
| Edit | 1.25 | CBH | eno2 | chrIII_ | Replace- | AGTG | 119 | AGTG | 223 | 27 |
| 46 | 459 | 1 | promo- | ATAT | ment of | GCAC | | GCAC | | |
| | 9 | | ter | AA(187, | “ATAT | CAAG | | CAAG | | |
| | | | trans- | 192)G | AA” | CATg | | CATg | | |
| | | | crip- | AGGC | with | aggc | | aggc | | |
| | | | tion | G | “GAGG | gAAA | | gAAA | | |
| | | | factor | | CG” | AAAA | | AAAA | | |
| | | | bind- | | | AAGC | | AAGC | | |
| | | | ing | | | ATTA | | ATTA | | |
| | | | site | | | | | | | |
| | | | indel | | | | | | | |
| Edit | 1.28 | CBH | eno2 | chrIII_ | Replace- | GTTC | 120 | GTTC | 224 | 27 |
| 47 | 005 | 1 | promo- | TTAAT | ment of | ATCG | | ATCG | | |
| | 9 | | ter | TT(187, | “TTAAT | TGGT | | TGGT | | |
| | | | trans- | 193)C | TT” with | TCAc | | TCAc | | |
| | | | crip- | ATTCT | “CATTC | attc | | attc | | |
| | | | tion | | T” | tTTT | | tTTT | | |
| | | | factor | | | TTCT | | TTTC | | |
| | | | bind- | | | CCAT | | TCCA | | |
| | | | ing | | | TGCT | | TTGC | | |
| | | | site | | | | | | | |
| | | | indel | | | | | | | |
| Edit | 1.24 | CBH | eno2 | chrIII_ | Replace- | GTTC | 121 | GTTC | 225 | 27 |
| 48 | 228 | 1 | promo- | TTAAT | ment of | ATCG | | ATCG | | |
| | | | ter | TT(187, | “TTAAT | TGGT | | TGGT | | |
| | | | trans- | 193)T | TT” with | TCAt | | TCAt | | |
| | | | crip- | GTTTC | “TGTTT | gttt | | gttt | | |
| | | | tion | A | CA” | caTT | | caTT | | |
| | | | factor | | | TTTC | | TTTC | | |
| | | | bind- | | | TCCA | | TCCA | | |
| | | | ing | | | TTGC | | TTGC | | |
| | | | site | | | T | | T | | |
| | | | indel | | | | | | | |
| Edit | 1.28 | CBH | eno2 | chrIII_ | Replace- | CATT | 122 | CATT | 226 | 27 |
| 49 | 909 | 1 | promo- | TTGAT | ment of | GCTT | | GCTT | | |
| | 3 | | ter | C(187, | “TTGAT | TCTG | | TCTG | | |
| | | | trans- | 192)CC | C” with | GCTc | | GCTc | | |
| | | | crip- | GGGG | “CCGG | cggg | | cggg | | |
| | | | tion | | GG” | gTTA | | gTTA | | |
| | | | factor | | | CTAT | | CTAT | | |
| | | | bind- | | | CATT | | CATT | | |
| | | | ing | | | TGGA | | TGGA | | |
| | | | site | | | | | | | |
| | | | indel | | | | | | | |
| Edit | 1.28 | CBH | eno2 | chrIII_ | Replace- | CATT | 123 | CATT | 227 | 27 |
| 50 | 005 | 1 | promo- | TTGAT | ment of | GCTT | | GCTT | | |
| | 9 | | ter | C(187, | “TTGAT | TCTG | | TCTG | | |
| | | | trans- | 192)CC | C” with | GCTc | | GCTc | | |
| | | | crip- | CCAC | “CCCC | ccca | | ccca | | |
| | | | tion | | AC” | CTTA | | cTTA | | |
| | | | factor | | | CTAT | | CTAT | | |
| | | | bind- | | | CATT | | CATT | | |
| | | | ing | | | TGGA | | TGGA | | |
| | | | site | | | | | | | |
| | | | indel | | | | | | | |
| Edit | 1.27 | CBH | eno2 | chrIII_ | Replace- | GTTC | 124 | GTTC | 228 | 27 |
| 51 | 677 | 1 | promo- | TTAAT | ment of | ATCG | | ATCG | | |
| | 4 | | ter | TT(187, | “TTAAT | TGGT | | TGGT | | |
| | | | trans- | 193)A | TT” with | TCAa | | TCAa | | |
| | | | crip- | CCGC | “ACCG | ccgc | | ccgc | | |
| | | | tion | TTTT | CTTTT” | tttt | | tttt | | |
| | | | factor | | | TTTT | | TTTT | | |
| | | | bind- | | | TCTC | | TCTC | | |
| | | | ing | | | CATT | | CATT | | |
| | | | site | | | GCT | | GCT | | |
| | | | indel | | | | | | | |
| Edit | 1.21 | CBH | eno2 | chrIII_ | Replace- | TTGG | 125 | TTGG | 229 | 27 |
| 52 | 517 | 1 | promo- | TTAAT | ment of | TTCA | | TTCA | | |
| | 7 | | ter | TT(187, | “TTAAT | TCGT | | TCGT | | |
| | | | trans- | 193)T | TT” with | GGTg | | GGTT | | |
| | | | crip- | GACT | “TGACT | aAtg | | CAtg | | |
| | | | tion | C | C” | actc | | actc | | |
| | | | factor | | | TTTT | | TTTT | | |
| | | | bind- | | | TCTC | | TTCT | | |
| | | | ing | | | CATT | | CCAT | | |
| | | | site | | | GCT | | TGC | | |
| | | | indel | | | | | | | |
| Edit | 1.23 | CBH | eno2 | chrIII_ | Replace- | CATT | 126 | CATT | 230 | 27 |
| 53 | 981 | 1 | promo- | TTGAT | ment of | GCTT | | GCTT | | |
| | 6 | | ter | C(187, | “TTGAT | TCTG | | TCTG | | |
| | | | trans- | 192)AG | C” with | GCTa | | GCTa | | |
| | | | crip- | GGG | “AGGG | gggg | | gggg | | |
| | | | tion | | G” | TTAC | | CTTA | | |
| | | | factor | | | TATC | | CTAT | | |
| | | | bind- | | | ATTT | | CATT | | |
| | | | ing | | | GGA | | TGG | | |
| | | | site | | | | | | | |
| | | | indel | | | | | | | |
| Edit | 1.21 | CBH | eno2 | chrIII_ | Replace- | GTTC | 127 | GTTC | 231 | 27 |
| 54 | 599 | 1 | promo- | TTAAT | ment of | ATCG | | ATCG | | |
| | 9 | | ter | TT(187, | “TTAAT | TGGT | | TGGT | | |
| | | | trans- | 193)C | TT” with | TCAc | | TCAc | | |
| | | | crip- | GCCA | “CGCC | gcca | | gcca | | |
| | | | tion | CCTAT | ACCTA | ccta | | ccta | | |
| | | | factor | CATTT | TCATT | tcat | | tcat | | |
| | | | bind- | TT | TTT” | tttt | | tttt | | |
| | | | ing | | (SEQ ID | TTTT | | TTTT | | |
| | | | site | | NO: 288) | TCTC | | TCTC | | |
| | | | indel | | | CATT | | CATT | | |
| | | | | | | GCT | | GCT | | |
| Edit | 1.15 | CBH | eno2 | chrIII_ | Replace- | GTTC | 128 | GTTC | 232 | 27 |
| 55 | 933 | 1 | promo- | TTAAT | ment of | ATCG | | ATCG | | |
| | | | ter | TT(187, | “TTAAT | TGGT | | TGGT | | |
| | | | trans- | 193)C | TT” with | TCAc | | TCAc | | |
| | | | crip- | CCCA | “CCCC | ccca | | ccca | | |
| | | | tion | C | AC” | cTTT | | cTTT | | |
| | | | factor | | | TTCT | | TTTC | | |
| | | | bind- | | | CCAT | | TCCA | | |
| | | | ing | | | TGCT | | TTGC | | |
| | | | site | | | | | | | |
| | | | indel | | | | | | | |
| Edit | 1.63 | YTA | dele- | GTAA | Deletion | AGAG | 129 | AGGT | 233 | 28 |
| 56 | 253 | 6 | tion | GAAA | of | TACG | | ATGG | | |
| | | | | ATCA | “GTAA | AACG | | CTGC | | |
| | | | | ATGG | GAAAA | CCAG | | GCAG | | |
| | | | | AGGC | TCAAT | TTAG | | TTAG | | |
| | | | | AGCA | GGAGG | CCTG | | CCTG | | |
| | | | | GAAA | CAGCA | GTCT | | GTCT | | |
| | | | | TAGG | GAAAT | CAG | | CAG | | |
| | | | | TATG | AGGTA | | | | | |
| | | | | GCTG | TGGCT | | | | | |
| | | | | CGCA1 | GCGCA” | | | | | |
| | | | | 027----- | (SEQ | | | | | |
| | | | | --------- | ID NO: | | | | | |
| | | | | --------- | 289) | | | | | |
| | | | | --------- | | | | | | |
| | | | | --------- | | | | | | |
| | | | | --- | | | | | | |
| Edit | 1.17 | YDL | promo- | chrIV_ | Deletion | TTCA | 130 | TTCA | 234 | 29 |
| 57 | 200 | 124W | ter | GCTTC | of | TAAT | | TAAT | | |
| | 4 | | dele- | AGTA | “GCTTC | GTTC | | GTTC | | |
| | | | tion | GGGC | AGTAG | TCCa | | TCCG | | |
| | | | | GGTA | GGCGG | aggg | | CTTT | | |
| | | | | ACTTC | TAACT | aCAG | | CCAG | | |
| | | | | TTCA | TCTTC | TCGA | | TCGA | | |
| | | | | GAAG | AGAAG | ACAC | | ACAC | | |
| | | | | AGAA | AGAAC | ACAT | | ACAT | | |
| | | | | CGGA | GGACC | CACA | | CACA | | |
| | | | | CCCG1 | CG” | TTAC | | TTAC | | |
| | | | | 87------ | (SEQ ID | ACGA | | ACGA | | |
| | | | | --------- | NO: 290) | TCGT | | TCGT | | |
| | | | | --------- | at | ACAG | | ACAG | | |
| | | | | --------- | position | CAAT | | CAAT | | |
| | | | | --------- | 1954 of | CCAT | | GCTT | | |
| | | | | | SEQ ID | TATT | | CAGT | | |
| | | | | | NO: 321 | TCTG | | AGGG | | |
| | | | | | | CAA | | CGG | | |
| Edit | 1.23 | SUM | dele- | ACAA | Deletion | ACCG | 131 | ACCG | 235 | 16 |
| 58 | 303 | 1 | tion | ACGT | of | ACTG | | ACTG | | |
| | 7 | | | CTAA | “ACAA | CCGT | | CCGT | | |
| | | | | ATCC | ACGTC | AGTG | | AGTA | | |
| | | | | AAAA | TAAAT | GTAT | | CAAA | | |
| | | | | 909----- | CCAAA | TTTG | | CGTC | | |
| | | | | --------- | A” (SEQ | TGGA | | TAAA | | |
| | | | | ------ | ID NO: | ACC | | TCC | | |
| | | | | | 291) | | | | | |
| Edit | 1.16 | UBP8 | dele- | TAAT | Deletion | GAGT | 132 | TGTG | 236 | 30 |
| 59 | 804 | | tion | AATT | of | CAAA | | CACT | | |
| | 1 | | | GTAA | “TAAT | TTCA | | CGAT | | |
| | | | | AGTG | AATTG | TTCA | | AAAA | | |
| | | | | CGTTC | TAAAG | TTGT | | TTGT | | |
| | | | | TCCA | TGCGT | TCAT | | TCAT | | |
| | | | | GATA | TCTCC | GAAC | | GAAC | | |
| | | | | AATG | AGATA | TTT | | TTT | | |
| | | | | TTTTT | AATGT | | | | | |
| | | | | CATG | TTTTC | | | | | |
| | | | | TGCA | ATGTG | | | | | |
| | | | | CTCG | CACTC | | | | | |
| | | | | ATAA | GATAA | | | | | |
| | | | | A663--- | A” (SEQ | | | | | |
| | | | | --------- | ID NO: | | | | | |
| | | | | --------- | 292) | | | | | |
| | | | | --------- | | | | | | |
| | | | | --------- | | | | | | |
| | | | | --------- | | | | | | |
| | | | | ------- | | | | | | |
| Edit | 1.16 | BDF1 | dele- | TTTCC | Deletion | CTGC | 133 | ACGA | 237 | 31 |
| 60 | 962 | | tion | TCATC | of | ACAC | | TGTT | | |
| | 6 | | | TTCA | “TTTCC | AACG | | AGCA | | |
| | | | | GATG | TCATC | GGTA | | GCGA | | |
| | | | | ACGA | TTCAG | AAGT | | AAGT | | |
| | | | | TGTTA | ATGAC | GAAG | | GAAG | | |
| | | | | GCAG | GATGT | AAGA | | AAGA | | |
| | | | | CG216 | TAGCA | GTG | | GTG | | |
| | | | | 2-------- | GCG” | | | | | |
| | | | | --------- | (SEQ ID | | | | | |
| | | | | --------- | NO: 293) | | | | | |
| | | | | ------- | | | | | | |
| Edit | 1.11 | NUP | dele- | GCTC | Deletion | CTGC | 134 | AAAC | 238 | 32 |
| 61 | 017 | 100 | tion | ATTAT | of | CTCC | | AACA | | |
| | 8 | | | TTGG | “GCTC | ACTA | | CAGC | | |
| | | | | AAAC | ATTAT | CCGT | | TTCT | | |
| | | | | AACA | TTGGA | ACGA | | ACGA | | |
| | | | | CAGC | AACAA | CAGT | | CAGT | | |
| | | | | TTC15 | CACAG | ACCT | | ACCT | | |
| | | | | 68------ | CTTC” | TCC | | TCC | | |
| | | | | --------- | (SEQ ID | | | | | |
| | | | | --------- | NO: 294) | | | | | |
| | | | | ---- | | | | | | |
| Edit | 1.53 | PRE9 | knock- | TACG | Insertion | ATGG | 135 | ATGG | 239 | 33 |
| 62 | 884 | | out | ATTCC | of triple | GTTC | | GTTC | | |
| | 1 | | | 166TA | stop | CAGA | | CAGA | | |
| | | | | ATAA | codon at | AGAt | | AGAt | | |
| | | | | TAA | position | aata | | aata | | |
| | | | | | 166 | ataa | | ataa | | |
| | | | | | | AGGA | | AGGA | | |
| | | | | | | CAAC | | CAAC | | |
| | | | | | | AATT | | AATT | | |
| | | | | | | TTCa | | TTCT | | |
| | | | | | | gcCC | | CCCC | | |
| | | | | | | TGAG | | TGAG | | |
| | | | | | | GGAC | | GGAC | | |
| | | | | | | GTCT | | GTCT | | |
| | | | | | | A | | A | | |
| Edit | 1.20 | GCN | knock- | GATC | Insertion | CATC | 136 | CATC | 240 | 34 |
| 63 | 567 | 5 | out | ACTT | of triple | AGAT | | AGAT | | |
| | 7 | | | G178T | stop | TGAA | | TGAA | | |
| | | | | AATA | codon at | GAGt | | GAGt | | |
| | | | | ATAA | position | aata | | aata | | |
| | | | | | 178 | ataa | | ataa | | |
| | | | | | | GATg | | GATG | | |
| | | | | | | gcgc | | GAGC | | |
| | | | | | | gacc | | TACG | | |
| | | | | | | ACGG | | ACGG | | |
| | | | | | | ATCC | | ATCC | | |
| | | | | | | CGAA | | CGAA | | |
| | | | | | | GTT | | GTT | | |
| Edit | 1.15 | ATP2 | knock- | AATA | Insertion | ATGA | 137 | ATGA | 241 | 35 |
| 64 | 383 | 3 | out | AGAA | of triple | CGAT | | CGAT | | |
| | 3 | | | A166T | stop | GCGA | | GCGA | | |
| | | | | AATA | codon at | ACAt | | ACAt | | |
| | | | | ATAA | position | aata | | aata | | |
| | | | | | 166 | ataa | | ataa | | |
| | | | | | | AATA | | AATA | | |
| | | | | | | AGAG | | AGAG | | |
| | | | | | | TTCT | | TTCT | | |
| | | | | | | AAT | | AAT | | |
| Edit | 1.22 | UBP8 | knock- | CATA | Insertion | ATGA | 138 | ATGA | 242 | 30 |
| 65 | 097 | | out | TACA | of triple | GCAT | | GCAT | | |
| | 6 | | | G166T | stop | TTGT | | TTGT | | |
| | | | | AATA | codon at | CCAt | | CCAt | | |
| | | | | ATAA | position | aata | | aata | | |
| | | | | | 166 | ataa | | ataa | | |
| | | | | | | CAAG | | CAAG | | |
| | | | | | | TATT | | TATT | | |
| | | | | | | TCAG | | TCAG | | |
| | | | | | | AAT | | AAT | | |
| Edit | 1.18 | KTR | knock- | ATCC | Insertion | ATGG | 139 | ATGG | 243 | 36 |
| 66 | 867 | 1 | out | CAGC | of triple | CGAA | | CGAA | | |
| | 9 | | | T166T | stop | GATT | | GATT | | |
| | | | | AATA | codon at | ATGt | | ATGt | | |
| | | | | ATAA | position | aata | | aata | | |
| | | | | | 166 | ataa | | ataa | | |
| | | | | | | tctA | | AGCA | | |
| | | | | | | AGCA | | AGCA | | |
| | | | | | | GCCT | | GCCT | | |
| | | | | | | GTTT | | GTTT | | |
| | | | | | | AC | | AC | | |
| Edit | 1.19 | PMT | knock- | TCTAC | Insertion | ATGT | 140 | ATGT | 244 | 37 |
| 67 | 547 | 2 | out | CGGG | of triple | CCTC | | CCTC | | |
| | 8 | | | 166TA | stop | GTCT | | GTCT | | |
| | | | | ATAA | codon at | TCGt | | TCGt | | |
| | | | | TAA | position | aata | | aata | | |
| | | | | | 166 | ataa | | ataa | | |
| | | | | | | TACA | | TACA | | |
| | | | | | | GCAA | | GCAA | | |
| | | | | | | AAAC | | AAAC | | |
| | | | | | | AAT | | AAT | | |
| Edit | 1.12 | URA | knock- | GTTTC | Insertion | ATGA | 141 | ATGA | 245 | 38 |
| 68 | 663 | 7 | out | AGGT1 | of triple | AGTA | | AGTA | | |
| | 6 | | | 66TAA | stop | CGTT | | CGTT | | |
| | | | | TAAT | codon at | GTTt | | GTTt | | |
| | | | | AA | position | aata | | aata | | |
| | | | | | 166 | ataa | | ataa | | |
| | | | | | | GGTG | | GGTG | | |
| | | | | | | TCAT | | TCAT | | |
| | | | | | | TTCG | | TTCG | | |
| | | | | | | GGT | | GGT | | |
| Edit | 1.24 | RFM | termi- | CTTCT | Replace- | GACT | 142 | GACT | 246 | 39 |
| 69 | 965 | 1 | nator | TTGA | ment of | CTAC | | CTAC | | |
| | 3 | | swap | AGAA | “CTTCT | CCAA | | CCAA | | |
| | | | | GTAA | TTGAA | TAGt | | TAGt | | |
| | | | | ATAA | GAAGT | atat | | atat | | |
| | | | | ATAT | AAATA | aact | | aact | | |
| | | | | AAAT | AATAT | gtct | | gtct | | |
| | | | | AGAG | AAATA | agaa | | agaa | | |
| | | | | AGAA | GAGAG | ataa | | ataa | | |
| | | | | AT108 | AAAT” | agag | | agag | | |
| | | | | 4TATA | (SEQ ID | tatc | | tatc | | |
| | | | | TAAC | NO: 295) | atct | | atct | | |
| | | | | TGTCT | at | ttca | | ttca | | |
| | | | | AGAA | position | aaAT | | aaAT | | |
| | | | | ATAA | 1 of SEQ | GACG | | GACG | | |
| | | | | AGAG | ID NO: | TATC | | TATC | | |
| | | | | TATC | 311 with | AATA | | AATA | | |
| | | | | ATCTT | “TATAT | T | | T | | |
| | | | | TCAA | AACTG | | | | | |
| | | | | A | TCTAG | | | | | |
| | | | | | AAATA | | | | | |
| | | | | | AAGAG | | | | | |
| | | | | | TATCA | | | | | |
| | | | | | TCTTTC | | | | | |
| | | | | | AAA” | | | | | |
| | | | | | (SEQ ID | | | | | |
| | | | | | NO: 296) | | | | | |
| Edit | 1.29 | YHR | termi- | CTCC | Replace- | GTCC | 143 | GTCC | 247 | 40 |
| 70 | 646 | 182W | nator | ATGC | ment of | CTTC | | CTTC | | |
| | 3 | | swap | ATGC | “CTCCA | TACA | | TACA | | |
| | | | | TACA | TGCAT | TAAt | | TAAt | | |
| | | | | TAGT | GCTAC | atat | | atat | | |
| | | | | AACT | ATAGT | aact | | aact | | |
| | | | | ACGT | AACTA | gtct | | gtct | | |
| | | | | AAAT | CGTAA | agaa | | agaa | | |
| | | | | CACC | ATCAC | ataa | | ataa | | |
| | | | | TGC25 | CTGC” | agag | | agag | | |
| | | | | 09TAT | (SEQ ID | tatc | | tatc | | |
| | | | | ATAA | NO: 297) | atct | | atct | | |
| | | | | CTGTC | with | ttca | | ttca | | |
| | | | | TAGA | “TATAT | aaTA | | aaTA | | |
| | | | | AATA | AACTG | CCTC | | CCTC | | |
| | | | | AAGA | TCTAG | TCTG | | TCTG | | |
| | | | | GTAT | AAATA | TTCT | | TTCT | | |
| | | | | CATCT | AAGAG | T | | T | | |
| | | | | TTCA | TATCA | | | | | |
| | | | | AA | TCTTTC | | | | | |
| | | | | | AAA” | | | | | |
| | | | | | (SEQ ID | | | | | |
| | | | | | NO: 298) | | | | | |
| | | | | | at | | | | | |
| | | | | | position | | | | | |
| | | | | | 1 of SEQ | | | | | |
| | | | | | ID NO: | | | | | |
| | | | | | 312 | | | | | |
| Edit | 1.30 | MEH | termi- | TGAA | Replace- | ACGG | 144 | ACGG | 248 | 41 |
| 71 | 166 | 1 | nator | CTTTT | ment of | TTCC | | TTCC | | |
| | 4 | | swap | TGTAT | “TGAA | CTTT | | CTTT | | |
| | | | | AACA | CTTTTT | TAAt | | TAAt | | |
| | | | | TCATT | GTATA | atat | | atat | | |
| | | | | GGTA | ACATC | aact | | aact | | |
| | | | | TACA | ATTGG | gtct | | gtct | | |
| | | | | AGCT | TATAC | agaa | | agaa | | |
| | | | | TTAT7 | AAGCT | ataa | | ataa | | |
| | | | | 00TAT | TTAT” | agag | | agag | | |
| | | | | ATAA | (SEQ ID | tatc | | tatc | | |
| | | | | CTGTC | NO: 299) | atct | | atct | | |
| | | | | TAGA | with | ttca | | ttca | | |
| | | | | AATA | “TATAT | aaAA | | aaAA | | |
| | | | | AAGA | AACTG | ATAA | | ATAA | | |
| | | | | GTAT | TCTAG | ATGT | | ATGT | | |
| | | | | CATCT | AAATA | TAAA | | TAAA | | |
| | | | | TTCA | AAGAG | T | | T | | |
| | | | | AA | TATCA | | | | | |
| | | | | | TCTTTC | | | | | |
| | | | | | AAA” | | | | | |
| | | | | | (SEQ ID | | | | | |
| | | | | | NO: 300) | | | | | |
| | | | | | at | | | | | |
| | | | | | position | | | | | |
| | | | | | 1 of SEQ | | | | | |
| | | | | | ID NO: | | | | | |
| | | | | | 313 | | | | | |
| Edit | 1.19 | YBR | termi- | TTATC | Replace- | TCGA | 145 | TCGA | 249 | 42 |
| 72 | 330 | 242W | nator | ATAA | ment of | TAAC | | TAAC | | |
| | 8 | | swap | ATAC | “TTATC | TAAA | | TAAA | | |
| | | | | CAAC | ATAAA | TAAt | | TAAt | | |
| | | | | TTTTG | TACCA | atat | | atat | | |
| | | | | CGTC | ACTTT | aact | | aact | | |
| | | | | ATAA | TGCGT | gtct | | gtct | | |
| | | | | AAGT | CATAA | agaa | | agaa | | |
| | | | | ACAA | AAGTA | ataa | | ataa | | |
| | | | | A868T | CAAA” | agag | | agag | | |
| | | | | ATAT | (SEQ ID | tatc | | tatc | | |
| | | | | AACT | NO: 301) | atct | | atct | | |
| | | | | GTCT | with | ttca | | ttca | | |
| | | | | AGAA | “TATAT | aaGT | | aaGT | | |
| | | | | ATAA | AACTG | AACT | | AACT | | |
| | | | | AGAG | TCTAG | ACCA | | ACCA | | |
| | | | | TATC | AAATA | ATAC | | ATAC | | |
| | | | | ATCTT | AAGAG | A | | A | | |
| | | | | TCAA | TATCA | | | | | |
| | | | | A | TCTTTC | | | | | |
| | | | | | AAA” | | | | | |
| | | | | | (SEQ ID | | | | | |
| | | | | | NO: 302) | | | | | |
| | | | | | at | | | | | |
| | | | | | position | | | | | |
| | | | | | 1 of SEQ | | | | | |
| | | | | | ID NO: | | | | | |
| | | | | | 314 | | | | | |
| Edit | 1.19 | PTK2 | termi- | ACGT | Replace- | TTTA | 146 | TTTA | 250 | 43 |
| 73 | 330 | | nator | TAGG | ment of | TCTC | | TCTC | | |
| | 8 | | swap | ACTTC | “ACGTT | AAGA | | AAGA | | |
| | | | | TTTAA | AGGAC | TAGt | | TAGt | | |
| | | | | TTCCC | TTCTTT | atat | | atat | | |
| | | | | TCTTT | AATTC | aact | | aact | | |
| | | | | TATG | CCTCT | gtct | | gtct | | |
| | | | | CTTTA | TTTAT | agaa | | agaa | | |
| | | | | GT260 | GCTTT | ataa | | ataa | | |
| | | | | 8TATA | AGT” | agag | | agag | | |
| | | | | TAAC | (SEQ ID | tatc | | tatc | | |
| | | | | TGTCT | NO: 303) | atct | | atct | | |
| | | | | AGAA | with | ttca | | ttca | | |
| | | | | ATAA | “TATAT | aaAT | | aaAT | | |
| | | | | AGAG | AACTG | CGTC | | CGTC | | |
| | | | | TATC | TCTAG | ATAT | | ATAT | | |
| | | | | ATCTT | AAATA | TCTT | | TCTT | | |
| | | | | TCAA | AAGAG | T | | T | | |
| | | | | A | TATCA | | | | | |
| | | | | | TCTTTC | | | | | |
| | | | | | AAA” | | | | | |
| | | | | | (SEQ ID | | | | | |
| | | | | | NO: 304) | | | | | |
| | | | | | at | | | | | |
| | | | | | position | | | | | |
| | | | | | 1 of SEQ | | | | | |
| | | | | | ID NO: | | | | | |
| | | | | | 315 | | | | | |
| Edit | 1.19 | YLR | termi- | AGCG | Replace- | GTGC | 147 | GTGC | 251 | 44 |
| 74 | 937 | 406C- | nator | CAGC | ment of | CGTT | | CGTT | | |
| | 6 | A | swap | CAGA | “AGCG | TAAA | | TAAA | | |
| | | | | TGCG | CAGCC | TGAt | | TGAt | | |
| | | | | ACAA | AGATG | atat | | atat | | |
| | | | | AAAC | CGACA | aact | | aact | | |
| | | | | TTAA | AAAAC | gtct | | gtct | | |
| | | | | AGGC | TTAAA | agaa | | agaa | | |
| | | | | GCGG | GGCGC | ataa | | ataa | | |
| | | | | CTC30 | GGCTC” | agag | | agag | | |
| | | | | 1TATA | (SEQ | tatc | | tatc | | |
| | | | | TAAC | ID NO: | atct | | atct | | |
| | | | | TGTCT | 305) | ttca | | ttca | | |
| | | | | AGAA | with | aaAG | | aaAG | | |
| | | | | ATAA | “TATAT | CCTA | | CCTA | | |
| | | | | AGAG | AACTG | GATT | | GATT | | |
| | | | | TATC | TCTAG | ACGT | | ACGT | | |
| | | | | ATCTT | AAATA | T | | T | | |
| | | | | TCAA | AAGAG | | | | | |
| | | | | A | TATCA | | | | | |
| | | | | | TCTTTC | | | | | |
| | | | | | AAA” | | | | | |
| | | | | | (SEQ ID | | | | | |
| | | | | | NO: 306) | | | | | |
| | | | | | at | | | | | |
| | | | | | position | | | | | |
| | | | | | 1 of SEQ | | | | | |
| | | | | | ID NO: | | | | | |
| | | | | | 316 | | | | | |
| Edit | 1.11 | STM | termi- | GCCTT | Replace- | TCTA | 148 | TCTA | 252 | 7 |
| 75 | 875 | 1 | nator | ATAT | ment of | ACTT | | ACTT | | |
| | 9 | | swap | ATGA | “GCCTT | GCCA | | GCCA | | |
| | | | | ATAA | ATATA | TCTc | | TCTT | | |
| | | | | TTCCA | TGAAT | ttgc | | TGGC | | |
| | | | | ACTG | AATTC | gtga | | TTAA | | |
| | | | | AAAG | CAACT | tata | | tata | | |
| | | | | AATC | GAAAG | taac | | taac | | |
| | | | | CAAT | AATCC | tgtc | | tgtc | | |
| | | | | A973T | AATA” | taga | | taga | | |
| | | | | ATAT | (SEQ ID | aata | | aata | | |
| | | | | AACT | NO: 307) | aaga | | aaga | | |
| | | | | GTCT | with | gtat | | gtat | | |
| | | | | AGAA | “TATAT | catc | | catc | | |
| | | | | ATAA | AACTG | tttc | | tttc | | |
| | | | | AGAG | TCTAG | aaaA | | aaaA | | |
| | | | | TATC | AAATA | CAGT | | CAGT | | |
| | | | | ATCTT | AAGAG | GTTC | | GTTC | | |
| | | | | TCAA | TATCA | TACT | | TACT | | |
| | | | | A | TCTTTC | TT | | TT | | |
| | | | | | AAA” | | | | | |
| | | | | | (SEQ ID | | | | | |
| | | | | | NO: 308) | | | | | |
| | | | | | at | | | | | |
| | | | | | position | | | | | |
| | | | | | 1 of SEQ | | | | | |
| | | | | | ID NO: | | | | | |
| | | | | | 317 | | | | | |
| Edit | 1.14 | GSH | termi- | ACTC | Replace- | GAAA | 149 | GAAA | 253 | 45 |
| 76 | 649 | 1 | nator | CTTTT | ment of | GCAA | | GCAA | | |
| | 8 | | swap | ACTTC | “ACTCC | ATGT | | ATGT | | |
| | | | | GGTT | TTTTA | TAAt | | TAAt | | |
| | | | | GTGA | CTTCG | atat | | atat | | |
| | | | | AAGA | GTTGT | aact | | aact | | |
| | | | | AAGT | GAAAG | gtct | | gtct | | |
| | | | | TGAC | AAAGT | agaa | | agaa | | |
| | | | | ATTAT | TGACA | ataa | | ataa | | |
| | | | | 2188T | TTAT” | agag | | agag | | |
| | | | | ATAT | (SEQ ID | tatc | | tatc | | |
| | | | | AACT | NO: 309) | atct | | atct | | |
| | | | | GTCT | with | ttca | | ttca | | |
| | | | | AGAA | “TATAT | aaCG | | aaCG | | |
| | | | | ATAA | AACTG | ATTT | | ATTT | | |
| | | | | AGAG | TCTAG | GGGT | | GGGT | | |
| | | | | TATC | AAATA | GACA | | GACA | | |
| | | | | ATCTT | AAGAG | C | | C | | |
| | | | | TCAA | TATCA | | | | | |
| | | | | A | TCTTTC | | | | | |
| | | | | | AAA” | | | | | |
| | | | | | (SEQ ID | | | | | |
| | | | | | NO: 310) | | | | | |
| | | | | | at | | | | | |
| | | | | | position | | | | | |
| | | | | | 1 of SEQ | | | | | |
| | | | | | ID NO: | | | | | |
| | | | | | 318 | | | | | |
| Edit | | ADA | knock- | T15*** | Triple | TGTT | 150 | TGTT | 254 | 46 |
| 77 | | 2 | out | | stop | CAGC | | CAGC | | |
| | | | | | codon | TGAT | | TGAT | | |
| | | | | | inserted | TGCt | | TGCt | | |
| | | | | | at the | aata | | aata | | |
| | | | | | T15 | ataa | | ataa | | |
| | | | | | residue | GTGA | | GTGA | | |
| | | | | | position | GGGT | | GGGT | | |
| | | | | | of SEQ | TTCA | | TTCA | | |
| | | | | | ID NO: | TGT | | TGT | | |
| | | | | | 46 | | | | | |
| Edit | | SCD6 | knock- | T15*** | Triple | TCTT | 151 | TCTT | 255 | 47 |
| 78 | | | out | | stop | TAAT | | TAAT | | |
| | | | | | codon | CTCT | | CTCT | | |
| | | | | | inserted | GTGt | | GTGt | | |
| | | | | | at the | aata | | aata | | |
| | | | | | T15 | ataa | | ataa | | |
| | | | | | residue | AGAT | | AGAT | | |
| | | | | | position | ATGT | | ATGT | | |
| | | | | | of SEQ | GGGG | | GGGG | | |
| | | | | | ID NO: | CTG | | CTG | | |
| | | | | | 47 | | | | | |
| Edit | | THI2 | knock- | L15*** | Triple | ACGC | 152 | ACGC | 256 | 48 |
| 79 | | 0 | out | | stop | CTCC | | CTCC | | |
| | | | | | codon | ACCA | | ACCA | | |
| | | | | | inserted | TATt | | TATt | | |
| | | | | | at the | aata | | aata | | |
| | | | | | L15 | ataa | | ataa | | |
| | | | | | residue | GCCT | | GCCT | | |
| | | | | | position | GCAA | | GCAA | | |
| | | | | | of SEQ | CGAA | | CGAA | | |
| | | | | | ID NO: | AAG | | AAG | | |
| | | | | | 48 | | | | | |
| Edit | | NUP | knock- | Y15*** | Triple | GTTG | 153 | GTTG | 257 | 49 |
| 80 | | 2 | out | | stop | CCGA | | CCGA | | |
| | | | | | codon | TGCG | | TGCG | | |
| | | | | | inserted | CAAa | | CAAA | | |
| | | | | | at the | ttca | | TACA | | |
| | | | | | Y15 | acgt | | GAGA | | |
| | | | | | residue | GAAA | | GAAA | | |
| | | | | | position | CGta | | CGta | | |
| | | | | | of SEQ | ataa | | ataa | | |
| | | | | | ID NO: | taaA | | taaA | | |
| | | | | | 49 | ACGA | | ACGA | | |
| | | | | | | GTCT | | GTCT | | |
| | | | | | | GACG | | GACG | | |
| | | | | | | AT | | AT | | |
| Edit | | CSS2 | knock- | V15*** | Triple | ATTT | 154 | ATTT | 258 | 50 |
| 81 | | | out | | stop | TTGT | | TTGT | | |
| | | | | | codon | TTCC | | TTCC | | |
| | | | | | inserted | TTCt | | TTCt | | |
| | | | | | at the | aata | | aata | | |
| | | | | | V15 | ataa | | ataa | | |
| | | | | | residue | TTTG | | TTTG | | |
| | | | | | position | CACA | | CACA | | |
| | | | | | of SEQ | TAAG | | TAAG | | |
| | | | | | ID NO: | CTC | | CTC | | |
| | | | | | 50 | | | | | |
| Edit | | SYO | knock- | L15*** | Triple | GCAT | 155 | GCAT | 259 | 51 |
| 82 | | 1 | out | | stop | CTTC | | CTTC | | |
| | | | | | codon | GTCT | | GTCT | | |
| | | | | | inserted | CGAt | | CGAt | | |
| | | | | | at the | aata | | aata | | |
| | | | | | L15 | ataa | | ataa | | |
| | | | | | residue | CTAC | | CTAC | | |
| | | | | | position | GCAA | | GCAA | | |
| | | | | | of SEQ | AGCT | | AGCT | | |
| | | | | | ID NO: | GGG | | GGG | | |
| | | | | | 51 | | | | | |
| Edit | | GLN | knock- | N15*** | Triple | CTGT | 156 | CTGT | 260 | 52 |
| 83 | | 3 | out | | stop | ACGA | | ACGA | | |
| | | | | | codon | CCTG | | CCTG | | |
| | | | | | inserted | CTGt | | CTGt | | |
| | | | | | at the | aata | | aata | | |
| | | | | | N15 | ataa | | ataa | | |
| | | | | | residue | CTGG | | CTGG | | |
| | | | | | position | ACGT | | ACGT | | |
| | | | | | of SEQ | GCAT | | GCAT | | |
| | | | | | ID NO: | GGTc | | GGTC | | |
| | | | | | 52 | gttc | | GAAG | | |
| | | | | | | tAAT | | TAAT | | |
| | | | | | | GAAG | | GAAG | | |
| | | | | | | AGCC | | AGCC | | |
| | | | | | | GAGA | | GAGA | | |
| Edit | | MGA | knock- | L15*** | Triple | CTTC | 157 | CTTC | 261 | 53 |
| 84 | | 1 | out | | stop | ATGC | | ATGC | | |
| | | | | | codon | AATT | | AATT | | |
| | | | | | inserted | CTTt | | CTTt | | |
| | | | | | at the | aata | | aata | | |
| | | | | | L15 | ataa | | ataa | | |
| | | | | | residue | GAAG | | GAAG | | |
| | | | | | position | TAAA | | TAAA | | |
| | | | | | of SEQ | TAAA | | TAAA | | |
| | | | | | ID NO: | TGG | | TGG | | |
| | | | | | 53 | | | | | |
| Edit | | RPS8 | knock- | G15*** | Triple | AAAA | 158 | AAAA | 262 | 54 |
| 85 | | B | out | | stop | GATC | | GATC | | |
| | | | | | codon | AGCC | | AGCC | | |
| | | | | | inserted | ACTt | | ACTt | | |
| | | | | | at the | aata | | aata | | |
| | | | | | G15 | ataa | | ataa | | |
| | | | | | residue | CGTG | | CGTG | | |
| | | | | | position | CTCA | | CTCA | | |
| | | | | | of SEQ | ATTC | | ATTC | | |
| | | | | | ID NO: | AGA | | AGA | | |
| | | | | | 54 | | | | | |
| Edit | | MSH | knock- | S15*** | Triple | TCGG | 159 | TCGG | 263 | 55 |
| 86 | | 5 | out | | stop | AAAC | | AAAC | | |
| | | | | | codon | AATG | | AATG | | |
| | | | | | inserted | AGAt | | AGAt | | |
| | | | | | at the | aata | | aata | | |
| | | | | | S15 | ataa | | ataa | | |
| | | | | | residue | aacg | | AATG | | |
| | | | | | position | aaga | | AGGA | | |
| | | | | | of SEQ | gGGT | | AGGT | | |
| | | | | | ID NO: | TTGC | | TTGC | | |
| | | | | | 55 | GGGA | | GGGA | | |
| | | | | | | TAAA | | TAAA | | |
| Edit | | SKI3 | knock- | L15*** | Triple | GAAG | 160 | GAAG | 264 | 56 |
| 87 | | | out | | stop | CCAA | | CCAA | | |
| | | | | | codon | ACAA | | ACAA | | |
| | | | | | inserted | GAAt | | GAAt | | |
| | | | | | at the | aata | | aata | | |
| | | | | | L15 | ataa | | ataa | | |
| | | | | | residue | cgcg | | CGTG | | |
| | | | | | position | atta | | ACTA | | |
| | | | | | of SEQ | cGAA | | TGAA | | |
| | | | | | ID NO: | GAGA | | GAGA | | |
| | | | | | 56 | CCAT | | CCAT | | |
| | | | | | | CGAA | | CGAA | | |
| Edit | | FSF1 | knock- | R15*** | Triple | GATT | 161 | GATT | 265 | 57 |
| 88 | | | out | | stop | TGCC | | TGCC | | |
| | | | | | codon | CGAA | | CGAA | | |
| | | | | | inserted | TCCt | | TCCt | | |
| | | | | | at the | aata | | aata | | |
| | | | | | R15 | ataa | | ataa | | |
| | | | | | residue | TTAT | | TTAT | | |
| | | | | | position | CCAC | | CCAC | | |
| | | | | | of SEQ | GTAT | | GTAT | | |
| | | | | | ID NO: | TGG | | TGG | | |
| | | | | | 57 | | | | | |
| Edit | | DUF | knock- | Y15*** | Triple | CTAA | 162 | CTAA | 266 | 58 |
| 89 | | 1 | out | | stop | TTTC | | TTTC | | |
| | | | | | codon | CCCA | | CCCA | | |
| | | | | | inserted | GATt | | GATt | | |
| | | | | | at the | aata | | aata | | |
| | | | | | Y15 | ataa | | ataa | | |
| | | | | | residue | TCCC | | TCCC | | |
| | | | | | position | AAGA | | AAGA | | |
| | | | | | of SEQ | TGCG | | TGCG | | |
| | | | | | ID NO: | CAC | | CAC | | |
| | | | | | 58 | | | | | |
| Edit | | SKI8 | knock- | H15*** | Triple | TTTA | 163 | TTTA | 267 | 59 |
| 90 | | | out | | stop | TTGC | | TTGC | | |
| | | | | | codon | CACA | | CACA | | |
| | | | | | inserted | GCAa | | GCAA | | |
| | | | | | at the | acgc | | ATGC | | |
| | | | | | H15 | gggc | | AGGT | | |
| | | | | | residue | aagG | | AAAG | | |
| | | | | | position | CTta | | CTta | | |
| | | | | | of SEQ | ataa | | ataa | | |
| | | | | | ID NO: | taaG | | taaG | | |
| | | | | | 59 | ATAT | | ATAT | | |
| | | | | | | TTTC | | TTTC | | |
| | | | | | | TCGG | | TCGG | | |
| | | | | | | TT | | TT | | |
| Edit | | EAP1 | knock- | S15*** | Triple | TCAA | 164 | TCAA | 268 | 60 |
| 91 | | | out | | stop | GCAG | | GCAG | | |
| | | | | | codon | CCAG | | CCAG | | |
| | | | | | inserted | TTTt | | TTTt | | |
| | | | | | at the | aata | | aata | | |
| | | | | | S15 | ataa | | ataa | | |
| | | | | | residue | TTGT | | TTGT | | |
| | | | | | position | CTGA | | CTGA | | |
| | | | | | of SEQ | CAGC | | CAGC | | |
| | | | | | ID NO: | GAT | | GAT | | |
| | | | | | 60 | | | | | |
| Edit | | PSH1 | knock- | G15*** | Triple | CTTC | 165 | CTTC | 269 | 61 |
| 92 | | | out | | stop | ACCA | | ACCA | | |
| | | | | | codon | AAAC | | AAAC | | |
| | | | | | inserted | GATt | | GATt | | |
| | | | | | at the | aata | | aata | | |
| | | | | | G15 | ataa | | ataa | | |
| | | | | | residue | GACG | | GACG | | |
| | | | | | position | CCAT | | CCAT | | |
| | | | | | of SEQ | ACTT | | ACTT | | |
| | | | | | ID NO: | TAT | | TAT | | |
| | | | | | 61 | | | | | |
| Edit | | MAG | knock- | G15*** | Triple | GCAA | 166 | GCAA | 270 | 62 |
| 93 | | 2 | out | | stop | GTGG | | GTGG | | |
| | | | | | codon | TGGC | | TGGC | | |
| | | | | | inserted | AGTt | | AGTt | | |
| | | | | | at the | aata | | aata | | |
| | | | | | G15 | ataa | | ataa | | |
| | | | | | residue | GAGA | | GAGA | | |
| | | | | | position | TGGA | | TGGA | | |
| | | | | | of SEQ | TACT | | TACT | | |
| | | | | | ID NO: | TTA | | TTA | | |
| | | | | | 62 | | | | | |
| Edit | | STE1 | knock- | L15*** | Triple | AGGA | 167 | AGGA | 271 | 63 |
| 94 | | 3 | out | | stop | AGAA | | AGAA | | |
| | | | | | codon | TAGT | | TAGT | | |
| | | | | | inserted | CATt | | CATt | | |
| | | | | | at the | aata | | aata | | |
| | | | | | L15 | ataa | | ataa | | |
| | | | | | residue | CAAA | | CAAA | | |
| | | | | | position | GGAA | | GGAA | | |
| | | | | | of SEQ | AAGT | | AAGT | | |
| | | | | | ID NO: | TCA | | TCA | | |
| | | | | | 63 | | | | | |
| Edit | | UBP1 | knock- | P15*** | Triple | ATCG | 168 | ATCG | 272 | 64 |
| 95 | | 5 | out | | stop | GTAC | | GTAC | | |
| | | | | | codon | TGTG | | TGTG | | |
| | | | | | inserted | TTTt | | TTTt | | |
| | | | | | at the | aata | | aata | | |
| | | | | | P15 | ataa | | ataa | | |
| | | | | | residue | CCCA | | CCCA | | |
| | | | | | position | TAGA | | TAGA | | |
| | | | | | of SEQ | TAAG | | TAAG | | |
| | | | | | ID NO: | AGC | | AGC | | |
| | | | | | 64 | | | | | |
| Edit | | SSK1 | knock- | D15*** | Triple | GTTT | 169 | GTTT | 273 | 65 |
| 96 | | | out | | stop | GGCT | | GGCT | | |
| | | | | | codon | ACGA | | ACGA | | |
| | | | | | inserted | ATAt | | ATAt | | |
| | | | | | at the | aata | | aata | | |
| | | | | | D15 | ataa | | ataa | | |
| | | | | | residue | ACTG | | ACTG | | |
| | | | | | position | ATGA | | ATGA | | |
| | | | | | of SEQ | AGTA | | AGTA | | |
| | | | | | ID NO: | AAC | | AAC | | |
| | | | | | 65 | | | | | |
| Edit | | YBR | knock- | D15*** | Triple | CAGG | 170 | CAGG | 274 | 66 |
| 97 | | 197C | out | | stop | TGAA | | TGAA | | |
| | | | | | codon | AGTA | | AGTA | | |
| | | | | | inserted | AGCt | | AGCt | | |
| | | | | | at the | aata | | aata | | |
| | | | | | D15 | ataa | | ataa | | |
| | | | | | residue | TCCA | | TCCA | | |
| | | | | | position | CCAA | | CCAA | | |
| | | | | | of SEQ | CAGA | | CAGA | | |
| | | | | | ID NO: | AGG | | AGG | | |
| | | | | | 66 | | | | | |
| Edit | | EGO | knock- | F15*** | Triple | AAGG | 171 | AAGG | 275 | 67 |
| 98 | | 2 | out | | stop | GAAC | | GAAC | | |
| | | | | | codon | CATT | | CATT | | |
| | | | | | inserted | GCTt | | GCTt | | |
| | | | | | at the | aata | | aata | | |
| | | | | | F15 | ataa | | ataa | | |
| | | | | | residue | CACG | | CACG | | |
| | | | | | position | GTAA | | GTAA | | |
| | | | | | of SEQ | CGTT | | CGTT | | |
| | | | | | ID NO: | ATA | | ATA | | |
| | | | | | 67 | | | | | |
| Edit | | SOA | knock- | Q15*** | Triple | ATTG | 172 | ATTG | 276 | 68 |
| 99 | | 1 | out | | stop | TAGA | | TAGA | | |
| | | | | | codon | AAAG | | AAAG | | |
| | | | | | inserted | GCCt | | GCCt | | |
| | | | | | at the | aata | | aata | | |
| | | | | | Q15 | ataa | | ataa | | |
| | | | | | residue | GTGT | | GTGT | | |
| | | | | | position | CCGC | | CCGC | | |
| | | | | | of SEQ | AGAA | | AGAA | | |
| | | | | | ID NO: | AGC | | AGC | | |
| | | | | | 68 | | | | | |
| Edit | | LCB4 | knock- | G15*** | Triple | ATCT | 173 | ATCT | 277 | 69 |
| 100 | | | out | | stop | TGAC | | TGAC | | |
| | | | | | codon | CGAT | | CGAT | | |
| | | | | | inserted | GAAt | | GAAt | | |
| | | | | | at the | aata | | aata | | |
| | | | | | G15 | ataa | | ataa | | |
| | | | | | residue | ATCA | | ATCA | | |
| | | | | | position | AATC | | AATC | | |
| | | | | | of SEQ | GCAA | | GCAA | | |
| | | | | | ID NO: | TCA | | TCA | | |
| | | | | | 69 | | | | | |
| Edit | | RSF2 | knock- | I15*** | Triple | GCGC | 174 | GCGC | 278 | 70 |
| 101 | | | out | | stop | CTGC | | CTGC | | |
| | | | | | codon | ATTA | | ATTA | | |
| | | | | | inserted | TGCt | | TGCt | | |
| | | | | | at the | aata | | aata | | |
| | | | | | I15 | ataa | | ataa | | |
| | | | | | residue | GCGG | | GCGG | | |
| | | | | | position | CCGC | | CCGC | | |
| | | | | | of SEQ | TCGA | | TCGA | | |
| | | | | | ID NO: | ATA | | ATA | | |
| | | | | | 70 | | | | | |
| Edit | | TCO | knock- | T15*** | Triple | TTGA | 175 | TTGA | 279 | 71 |
| 102 | | 89 | out | | stop | AGTC | | AGTC | | |
| | | | | | codon | AGAC | | AGAC | | |
| | | | | | inserted | ACTg | | ACTG | | |
| | | | | | at the | acgt | | ATGT | | |
| | | | | | T15 | gtaa | | Ataa | | |
| | | | | | residue | taat | | taat | | |
| | | | | | position | aaAA | | aaAA | | |
| | | | | | of SEQ | TGCG | | TGCG | | |
| | | | | | ID NO: | TCAA | | TCAA | | |
| | | | | | 71 | CAGT | | CAGT | | |
| | | | | | | A | | A | | |
| Edit | | HEM | knock- | R15*** | Triple | CTTT | 176 | CTTT | 280 | 72 |
| 103 | | 15 | out | | stop | CCAG | | CCAG | | |
| | | | | | codon | AACA | | AACA | | |
| | | | | | inserted | ATCc | | ATCC | | |
| | | | | | at the | gcac | | GTAC | | |
| | | | | | R15 | ccag | | ACAA | | |
| | | | | | residue | ggcT | | GGTT | | |
| | | | | | position | CCTT | | CCTT | | |
| | | | | | of SEQ | CCTA | | CCTA | | |
| | | | | | ID NO: | AGAt | | AGAt | | |
| | | | | | 72 | aata | | aata | | |
| | | | | | | ataa | | ataa | | |
| | | | | | | CTGA | | CTGA | | |
| | | | | | | CCAT | | CCAT | | |
| | | | | | | TACA | | TACA | | |
| | | | | | | AGA | | AGA | | |
| Edit | | POM | knock- | V15*** | Triple | TTGG | 177 | TTGG | 281 | 73 |
| 104 | | 34 | out | | stop | ACGA | | ACGA | | |
| | | | | | codon | TAAT | | TAAT | | |
| | | | | | inserted | GACt | | GACt | | |
| | | | | | at the | aata | | aata | | |
| | | | | | V15 | ataa | | ataa | | |
| | | | | | residue | ccgc | | CCAT | | |
| | | | | | position | tgCC | | TGCC | | |
| | | | | | of SEQ | GGAC | | GGAC | | |
| | | | | | ID NO: | ACAG | | ACAG | | |
| | | | | | 73 | ACAG | | ACAG | | |
| | | | | | | C | | C | | |

In addition, variants combining certain of these edits are listed in Table 2:

TABLE 2
Edit Combinations

| | | First | Second | Third |
|---|---|---|---|---|
| Edit Combination No. | Phenotype | Edit | Edit | Edit |
| Edit combination 1 | 2.36 | Edit 28 | Edit 77 | |
| Edit combination 2 | 2.12 | Edit 28 | Edit 2 | |
| Edit combination 3 | 1.95 | Edit 28 | Edit 6 | |
| Edit combination 4 | 1.88 | Edit 28 | Edit 78 | |
| Edit combination 5 | 1.84 | Edit 28 | Edit 79 | |
| Edit combination 6 | 2.04 | Edit 28 | Edit 19 | |
| Edit combination 7 | 2.03 | Edit 28 | Edit 80 | |
| Edit combination 8 | 1.9 | Edit 28 | Edit 81 | |
| Edit combination 9 | 1.9 | Edit 28 | Edit 82 | |
| Edit combination 10 | 1.93 | Edit 28 | Edit 83 | |
| Edit combination 11 | 1.77 | Edit 28 | Edit 84 | |
| Edit combination 12 | 1.84 | Edit 28 | Edit 85 | |
| Edit combination 13 | 1.76 | Edit 28 | Edit 86 | |
| Edit combination 14 | 1.94 | Edit 28 | Edit 87 | |
| Edit combination 15 | 1.88 | Edit 28 | Edit 78 | |
| Edit combination 16 | 1.75 | Edit 28 | Edit 88 | |
| Edit combination 17 | 1.8 | Edit 28 | Edit 89 | |
| Edit combination 18 | 1.92 | Edit 34 | Edit 90 | |
| Edit combination 19 | 2.08 | Edit 34 | Edit 91 | |
| Edit combination 20 | 1.86 | Edit 34 | Edit 92 | |
| Edit combination 21 | 1.74 | Edit 34 | Edit 93 | |
| Edit combination 22 | 1.61 | Edit 34 | Edit 94 | |
| Edit combination 23 | 1.92 | Edit 34 | Edit 2 | |
| Edit combination 24 | 1.68 | Edit 34 | Edit 95 | |
| Edit combination 25 | 1.97 | Edit 34 | Edit 96 | |
| Edit combination 26 | 1.63 | Edit 34 | Edit 97 | |
| Edit combination 27 | 1.66 | Edit 34 | Edit 98 | |
| Edit combination 28 | 1.62 | Edit 34 | Edit 99 | |
| Edit combination 29 | 1.64 | Edit 34 | Edit 5 | |
| Edit combination 30 | 1.66 | Edit 34 | Edit 100 | |
| Edit combination 31 | 1.8 | Edit 34 | Edit 2 | |
| Edit combination 32 | 1.79 | Edit 34 | Edit 19 | |
| Edit combination 33 | 1.86 | Edit 34 | Edit 11 | |
| Edit combination 34 | 2.02 | Edit 34 | Edit 62 | |
| Edit combination 35 | 1.84 | Edit 34 | Edit 43 | |
| Edit combination 36 | 2.74 | Edit 28 | Edit 77 | Edit 2 |
| Edit combination 37 | 2.62 | Edit 28 | Edit 77 | Edit 43 |
| Edit combination 38 | 2.68 | Edit 28 | Edit 77 | Edit 58 |
| Edit combination 39 | 2.8 | Edit 28 | Edit 77 | Edit 9 |
| Edit combination 40 | 2.66 | Edit 28 | Edit 77 | Edit 6 |
| Edit combination 41 | 2.84 | Edit 28 | Edit 77 | Edit 12 |
| Edit combination 42 | 3.08 | Edit 28 | Edit 77 | Edit 101 |
| Edit combination 43 | 3.13 | Edit 28 | Edit 77 | Edit 102 |
| Edit combination 44 | 2.97 | Edit 28 | Edit 77 | Edit 103 |
| Edit combination 45 | 2.79 | Edit 28 | Edit 77 | Edit 104 |

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A Saccharomyces cerevisiae cell comprising a transgene encoding a cellobiohydrolase I (CBHI) enzyme and at least one modification affecting the expression or activity of a protein, wherein a wildtype version of the protein comprises an amino acid sequence selected from the group consisting of SEQ ID NOs: 1 to 73.

2. The S. cerevisiae cell of claim 1, wherein a nucleic acid molecule comprising the at least one modification comprises a nucleic acid sequence selected from the group consisting of SEQ ID NOs: 74 to 281.

3. The S. cerevisiae cell of claim 1 or 2, wherein the S. cell exhibits enhanced CBHI activity as compared to a control cell lacking the at least one modification.

4. A Saccharomyces cerevisiae cell comprising a transgene encoding a cellobiohydrolase I (CBHI) enzyme and a null allele of a nucleic acid molecule encoding a protein, wherein a wildtype version of the protein comprises an amino acid sequence selected from the group consisting of SEQ ID NOs: 1 to 73.

5. A Saccharomyces cerevisiae cell comprising a transgene encoding a cellobiohydrolase I (CBHI) enzyme and at least one substitution allele of a nucleic acid molecule encoding a protein, wherein a wildtype version of the protein comprises an amino acid sequence selected from the group consisting of SEQ ID NOs: 1 to 73.

6. A Saccharomyces cerevisiae cell comprising a transgene encoding a cellobiohydrolase I (CBHI) enzyme and at least one synonymous edit of a nucleic acid molecule encoding a protein, wherein a wildtype version of the protein comprises an amino acid sequence selected from the group consisting of SEQ ID NOs: 1 to 73.

7. A Saccharomyces cerevisiae cell comprising a transgene encoding a cellobiohydrolase I (CBHI) enzyme and at least one regulatory element modification in a nucleic acid molecule encoding a protein, wherein a wildtype version of the protein comprises an amino acid sequence selected from the group consisting of SEQ ID NOs: 1 to 73.

8. The S. cerevisiae cell of claim 7, wherein the at least one regulatory element modification comprises a nucleic acid sequence selected from the group consisting of SEQ ID NOs: 101 to 105, 110 to 128, 130, 142 to 149, 205 to 209, 214 to 232, 234, and 246 to 253.

9. The S. cerevisiae cell of claim 7 to 8, wherein the S. cerevisiae cell exhibits enhanced CBHI activity as compared to a control cell lacking the at least one regulator element modification.

10. A Saccharomyces cerevisiae cell comprising a transgene encoding a cellobiohydrolase I (CBHI) enzyme and at least one insertion or deletion in a nucleic acid molecule encoding a protein, wherein a wildtype version of the protein comprises an amino acid sequence selected from the group consisting of SEQ ID NOs: 1 to 73.

11. A Saccharomyces cerevisiae cell comprising a transgene encoding a cellobiohydrolase I (CBHI) enzyme, a first modification affecting the expression or activity of a first protein, and a second modification affecting the expression or activity of a second protein, wherein a wildtype version of the first protein and a wildtype version of the second protein each comprise an amino acid sequence selected from the group consisting of SEQ ID NOs: 1 to 73.

12. The S. cerevisiae cell of any one of claims 1 to 11, wherein the transgene comprises a promoter operably linked to a nucleic acid sequence encoding the CBHI enzyme.

13. The S. cerevisiae cell of claim 12, wherein the promoter comprises SEQ ID NO: 319.

14. The S. cerevisiae cell of any one of claims 1 to 13, wherein the transgene comprises a terminator operably linked to a nucleic acid sequence encoding the CBHI enzyme.

15. The S. cerevisiae cell of any one of claims 1 to 14, wherein the transgene is codon optimized for S. cerevisiae.

16. The S. cerevisiae cell of any one of claims 1 to 15, wherein the transgene encodes a polypeptide comprising SEQ ID NO: 27.

17. A Saccharomyces cerevisiae cell comprising an Enolase-2 promoter sequence comprising a sequence selected from the group consisting of SEQ ID NOs: 101 to 105, 110 to 128, 205 to 209, and 214 to 232 operably linked to a nucleic acid sequence encoding a cellobiohydrolase I (CBHI) enzyme, wherein the nucleic acid sequence encoding CBHI comprises a sequence selected from the group consisting of SEQ ID NOs: 106 to 109, 100, 204, and 210 to 213.

18. A Saccharomyces cerevisiae cell comprising an Enolase-2 promoter sequence comprising a sequence selected from the group consisting of SEQ ID NOs: 101 to 105, 110 to 128, 205 to 209, and 214 to 232 operably linked to a nucleic acid sequence encoding a cellobiohydrolase I (CBHI) enzyme comprising an amino acid sequence set forth in SEQ ID NO: 27 with a substitution from glycine to valine at position 22.

19. A Saccharomyces cerevisiae cell comprising an edit listed in Table 1.

20. A Saccharomyces cerevisiae cell comprising an edit combination listed in Table 2.
